(19) United States
(12) Patent Application Publication
(10) Pub. No.: US 2018/0169230 A1
(43) Pub. Date: Jun. 21, 2018

(54) COMBINATION CANCER THERAPIES

(71) Applicant: Merrimack Pharmaceuticals, Inc., Cambridge, MA (US)

(72) Inventors: Sharlene ADAMS, Waltham, MA (US); Michael CURLEY, Boston, MA (US); Gregory J. FINN, Watertown, MA (US); Chrystal U. LOUIS, Somerville, MA (US); Alexey Alexandrovich LUGOVSKOY, Belmont, MA (US)

(21) Appl. No.: 15/577,382

(22) PCT Filed: May 27, 2016
(86) PCT No.: PCT/US2016/034846

§ 371 Date: Nov. 28, 2017

Related U.S. Application Data
(60) Provisional application No. 62/168,553, filed on May 29, 2015.
(60) Provisional application No. 62/245,825, filed on Oct. 23, 2015.

(51) Int. Cl. A61K 39/395; C07K 16/28; C07K 16/32; A61P 35/00

(52) U.S. Cl. CPC A61K 39/395; C07K 16/2827; A61K 2039/507; C07K 16/32; A61P 35/00; C07K 16/2863

(57) ABSTRACT

The disclosure provides methods of treatment for cancer in patients using bispecific anti-IGF-1R, anti-ErbB3 antibodies in combination with one or more therapeutic agents that impede regulatory T-cell agents. In certain embodiments, said one or more therapeutic agent may be an antagonistic anti-receptor antibody that immunospecifically binds human PD-L1.

### RELATED APPLICATIONS

This application claims priority to, and the benefit of, U.S. Provisional Patent Application Nos. 62/168,553 and 62/245,825, filed May 29, 2015 and Oct. 23, 2015, respectively. The entire content of the aforementioned patent applications are incorporated by reference in its entirety.

### SEQUENCE LISTING

The instant application contains a Sequence Listing which has been submitted electronically in ASCII format and is hereby incorporated by reference in its entirety. Said ASCII copy, created on May 27, 2016, is named MMJ_067PC_Sequence_Listing.txt and is 984,902 bytes in size.

### BACKGROUND

Tumor cells express receptors for growth factors and cytokines that stimulate proliferation of the cells. Antibodies to such receptors can be effective in blocking the stimulation of cell proliferation mediated by growth factors and cytokines and can thereby inhibit tumor cell proliferation and tumor growth. Commercially available therapeutic antibodies that target receptors on cancer cells include, for example, trastuzumab which targets the HER2 receptor (also known as ErbB2) for the treatment of breast cancer, and cetuximab which targets the epidermal growth factor receptor (EGFR, also known as HER1 or ErbB1) for the treatment of colorectal cancer and head and neck cancer. Antibodies in development include seribantumab, which targets the HER3 receptor (also known as ErbB3), which is being developed for the treatment of several types of cancer, and mapatumumab, which is an antibody against the TRAIL receptor.

Istiratumab (MM-141) is a fully human, tetravalent, bi-specific monoclonal antibody that binds to IGF-1R and ErbB3, and co-inhibits IGF-1R and ErbB3 signaling (disclosed, e.g., in U.S. Pat. No. 8,476,409 and herein). Using a Systems Biology approach, MM-141 was designed to be more active than mono-specific antibodies and their combinations at blocking IGF-1 and IGF-2 from binding to the IGF-1 receptor (IGF-1R), and heregulin (HRG) binding to its receptor ErbB3. These studies also demonstrated that simply inhibiting IGF-1R was not sufficient, since inhibiting IGF-1R leads to an increase in ErbB3 expression and thus allows ErbB3 to act as a compensation mechanism. Hence, dual blockade of this pathway is needed to effectively inhibit cancer cells that rely upon IGF-1 and/or ErbB3.

Immunotherapeutics have rapidly become an important option for patients and oncologists in the treatment of malignant diseases. Immunotherapy, at its most basic level, uses the immune system to fight cancer and disease. When the immune system is working properly, it is able to identify foreign and harmful components within the body and eliminate them. For example, when patients are infected with the flu virus or a bacterial infection, the immune system will recruit white blood cells and other immune cells to the site of the infection. Once there, the different components of the immune system work together to target the foreign viruses or bacteria and remove them from the body.

Cancer cells use a variety of mechanisms to remain invisible to the immune system. In some cases, the cancer cells down-regulate cell surface markers or proteins that would normally be used by the immune system to recognize the cells as being foreign and therefore targeted for destruction. Cancer cells and other cells within its microenvironment can also secrete soluble proteins, called cytokines or chemokines, which limit the function of immune cells. Furthermore, cancer cells can recruit other cells to make the immune system think they are normal or “host” cells that should not be destroyed.

One of the mechanisms that the body uses to prevent the immune system from attacking normal tissues and/or leading to autoimmune disease is to modulate the number of regulatory T cells (“Tregs”) in any location. Genetic mutations leading to severe decreases in either the number or function of Tregs have been reported to lead to autoimmune disease. However, cancer cells have modified this paradigm by recruiting Tregs to its microenvironment in order to make them invisible to the immune system. Many studies have noted that elevated levels of Tregs within the tumor microenvironment are associated with poor responses to therapy and poor overall survival. Immunotherapeutics targeting Tregs have led to improvements in clinical response rates, thus suggesting the importance of this pathway in a subset of cancer types.

Cancer immunotherapy, irrespective of treatment modality (i.e., adoptive cellular therapy, vaccines, monoclonal antibodies, and checkpoint inhibitors) and target (i.e., CD19, GD2, PD1, PD-L1, and T regs), has shown promise within a subset of patients. The goal is to expand the clinical utility of these agents, either alone or in combination, in order to provide all patients with the optimal chance of being cured of their cancer. Thus, there is an unmet need to discover not only therapeutics that target cancer cells, but also those that target cells within the microenvironment that allow cancer cells to evade detection by the immune system, e.g., Tregs. The disclosure below addresses this need.

### SUMMARY

Disclosed herein are methods for providing treatment for a cancer in a human patient. These methods comprise administration to the patient of an effective amount of a bispecific anti-IGF-1R/anti-ErbB3 antibody that exhibits immunospecific binding to the extracellular domain of human IGF-1R (CD221) and to human ErbB3 together with a therapy that can impede Treg activity. In preferred embodiments the binding to IGF-1R is characterized as inhibiting IGF-1 mediated activation of human IGF-1R. In other preferred embodiments the dosage and administration of the antibody are adapted for one or more of: a) the inhibition of proliferation in IGF-1R ligand-dependent and/or IGF-1R ligand-independent IGF-1R expressing immune cells; b) the inhibition of differentiation of IGF-1R expressing immune progenitor cells into T cells, NKT cells, or NK cells, and c) depletion of IGF-1R expressing immune cells. Such adaptation may include administration in combination with other antibodies or drugs or therapies, e.g., as described hereinbelow. Thus, the bispecific antibody may be administered as monotherapy or in combination with an effective amount of one or more of: a small molecule immunomodulatory agent, one or more antineoplastic agents (“chemotherapy”), radiation, an anticancer vaccine, and an immunomodulatory antibody that does not bind to human IGF-1.

In other embodiments, the immunomodulatory antibody that does not bind to human IGF-1 is one or more of: a) agonistic anti-receptor antibodies that immunospecifically bind to human OX40, CD40, GITR, CD27, ICOS, or 4-1BB; b) antagonistic anti-receptor antibodies that immunospecifically bind to: i) human CTLA-4 (optionally ipilimumab or tremelimumab), ii) PD-1 (optionally nivolumab, pembrolizumab, or pidilizumab), iii) PD-L1 (optionally atezolizumab, durvalumab, or avelumab), iv) TIM-3, BTLA, VISTA, LAG-3, KIR (optionally lirilumab), CD47, CD25, B7-H3, or B7-H4; and c) anti-ligand antibodies that block function of IL-6, IL-10, TGFβ, angiopoetin-2, VEGF, IL-17, IL-23, or TNFα. In various embodiments the anticancer vaccine is selected from the group consisting of OncoVex, MAGE-A3, PROSTVAC, GVAX, CDX110, CDX1307, CDX1401, CV9104, BIOVAXID, IMA 901 and ADXS11-001.

Preferred bispecific antibodies are selected from istiratumab (P4-G1-M1.3), SF-G1-P1, SF-G1-M1.3, SF-G1-M27, SF-G1-P6, SF-G1-B69, P4-G1-C8, P4-G1-P1, P4-G1-M27, P4-G1-P6, P4-G1-B69, M78-G1-C8, M78-G1-P1, M78-G1-M1.3, M78-G1-M27, M78-G1-P6, M78-G1-B69, M57-G1-C8, M57-G1-P1, M57-G1-M1.3, M57-G1-M27, M57-G1-P6, M57-G1-B69, P1-G1-P4, P1-G1-M57, P1-G1-M78, M27-G1-P4, M27-G1-M57, M27-G1-M78, M7-G1-P4, M7-G1-M57, M7-G1-M78, B72-G1-P4, B72-G1-M57, B72-G1-M78, B60-G1-P4, B60-G1-M57, B60-G1-M78, P4M-G1-M1.3, P4M-G1-C8, P33M-G1-M1.3, P33M-G1-C8, P4M-G1-P6L, P33M-G1-P6L, and P1-G1-M76; each of which is described in U.S. Pat. No. 8,476,409 and herein. In one embodiment, the bispecific antibody may be administered intravenously at one of the following schedules: 6 mg/kg qw, 9 mg/kg qw, 8 mg/kg qw, 10 mg/kg q2w, 12 mg/kg q3w, 18 mg/kg q3w, 20 mg/kg q3w, 30 mg/kg q3w, 20 mg/kg qw, or 40 mg/kg q2w. In another embodiment, the bispecific antibody may be administered intravenously at 2.8 g q2w, 2.24 g q2w, or 1.96 g q2w. Administration may be intravenous or subcutaneous.

In various embodiments the bispecific antibody has been engineered to increase or decrease binding to the Fc receptors. In others, the bispecific antibody exhibits preferential binding to regulatory T cells compared to CD8 positive (CD8+) cytotoxic T cells. Preferably, the regulatory T cells are CD4 positive (CD4+) and either or both of CD25 positive (CD25+) and FoxP3 positive (FoxP3+). In other embodiments, the bispecific antibody can counter the inhibitory effects of IGF-1 and heregulin ligands on plasmacytoid dendritic cell activity as well as inhibit the suppressive activity of myeloid-derived suppressive cells on CD4+ T cell proliferation, both of which may potentially improve the anti-tumor immune response.

The methods of treatment and other inventions disclosed herein are based in part on discoveries provided herein. For example, as outlined in the Examples below, the Applicants have discovered: (i) regulatory T cells and other immune cells express IGF-1R and ErbB3 on their surface; (ii) the ligand IGF-1 can stimulate Treg induction from CD4+ T cells and Treg proliferation, which can be inhibited by co-treatment with istiratumab in vitro; (iii) istiratumab monotherapy treatment has an improved anti-tumor effect in immune-compromised and immune-competent mouse models of cancer that do express or do not express IGF-1R and ErbB3 on their cell surface; and (iv) istiratumab can potentiate the activity of anti-PD-L1, anti-CTLA-4 or anti-PD-1 therapeutics in mouse models of cancer.

In accordance with another aspect, the patient to be treated using the methods disclosed herein is selected for treatment by assessing a biomarker level in a biopsy of the cancer and the patient is treated with the bispecific antibody if the biopsy exhibits a level that is predictive of a favorable response to the treatment. In various embodiments of this aspect, the biomarker is 1) fraction of IGF-1R positive regulatory T cells, 2) fraction of IGF-1R positive cytotoxic T cells, or 3) the ratio of 1) to 2); and the level that is predictive of a favorable response to the treatment is where 1) or 2) or 3) is higher than the fraction or ratio corresponding to a population median level of patients with the cancer. In other embodiments of this aspect, the biomarker is serum free IGF-1 and the level that is predictive of a favorable response to the treatment is where the patient's serum free IGF-1 concentration is higher than a population median level of patients with the cancer. In yet other embodiments of this aspect, the biomarker is biopsy heregulin level and the level that is predictive of a favorable response to the treatment is where the biopsy heregulin level is higher than a population median biopsy heregulin level of patients with the cancer; or the biomarker is biopsy heregulin level and the level that is predictive of a favorable response to the treatment is a biopsy heregulin level that is sufficient to be detectable by RT-PCR; or the biomarker is biopsy heregulin level and the level that is predictive of a favorable response to the treatment is a biopsy heregulin level that sufficient to be detectable by RNA-ISH.

In one embodiment, a therapeutically effective amount of istiratumab can be used (e.g., intravenously administered) as an immune-oncology monotherapy to treat colorectal cancer, melanoma, B-cell lymphoma or fibrosarcoma cancer in a human patient without a diagnosed active autoimmune condition. In another embodiment, the use of an antineoplastic therapy can consist of administering istiratumab in combination with an immunomodulatory agent selected from the group consisting of: an anti-PD-L1 antibody, an anti-CTLA-4 antibody and an anti-PD-1 antibody, to treat colorectal cancer, melanoma, B-cell lymphoma or fibrosarcoma cancer in a human patient having immune cells expressing IGF-1R and ErbB3 and without a diagnosed active autoimmune condition. In a third embodiment, a therapeutically effective amount of istiratumab can be used as an immune-oncology to treat a cancer that does not express at least one of IGF-1R and ErbB3 in a human patient without a diagnosed active autoimmune condition, and having immune cells expressing IGF-1R and ErbB3. In a fourth embodiment, an antineoplastic therapy consisting of istiratumab in combination with an immunomodulatory agent selected from the group consisting of: an anti-PD-L1 antibody, an anti-CTLA-4 antibody and an anti-PD-1 antibody, can be used to treat a cancer that does not express at least one of IGF-1R and ErbB3 in a human patient without a diagnosed active autoimmune condition, and having immune cells expressing IGF-1R and ErbB3 in a human patient without a diagnosed active autoimmune condition.

Preferably, the human patient can have immune cells that express IGF-1R and ErbB3. The human patient can have Treg cells expressing IGF-1R and ErbB3. The ligand IGF-1 can be present in the human patient (e.g., as serum free IGF-1 ligand, or elsewhere in the human such as in a tissue biopsy). A therapeutically effective amount of istiratumab can be administered once every week or once every two weeks. In some embodiments, the therapeutically effective amount of istiratumab is a fixed dose of 2.8 g, 2.24 g, or 1.96 g administered once every two weeks. In another embodiment, the therapeutically effective amount of istiratumab is 20 mg/kg administered once every week or 40 mg/kg administered once every two weeks.

### BRIEF DESCRIPTION OF THE DRAWINGS

FIG. 1 is a graph demonstrating the mean percent expression of IGF-1R positive (IGF-1R+) regulatory T cells (“Tregs”; here identified as CD4+CD25+FoxP3+ cells) and CD8+ effector T cells (“CD8”) in lymphocyte populations of human whole blood, as measured by quantitative flow cytometry.

FIG. 2 is a set of panels showing the levels of IGF-1R+ Tregs in lymphocyte populations of wild-type C57BL/6 mouse-derived splenocytes that have been stained by an anti-IGF-1R antibody compared to a non-IGF-1R-antibody-stained control, as measured by quantitative flow cytometry. The lower two panels comprise cells stained with an anti-IGF-1R antibody, and the top two panels are unstained (control) cells. In the left hand panels the dots in the inner large square represent CD4+CD25+(Treg) cells. To obtain the data in the right hand panels, the Treg cells from the left hand panels inner square were re-analyzed again; the dots in the inner top right square represent those of the CD4+CD25+ cells that are also IGF-1R+FoxP3+. These data show that almost half of the Treg cells express IGF-1R.

FIG. 3A and FIG. 3B are a series of panels showing the effect of IGF-1 on the induction of Tregs from CD4+ cells, as measured by flow cytometry (FIG. 3A) and the effect of IGF-1 on the proliferation of Tregs as evidenced by the number of population doublings using carboxyfluorescein succinimidyl ester (CFSE)-fluorescein isothiocyanate (FITC) staining analyzed by flow cytometry. The percentage of Treg cells increased from 0.908% (left panel) to 3.91% (right panel) of CD4+ cells treated with 0 and 200 ng/mL IGF-1, respectively (FIG. 3A). Analyses of proliferation measured by CFSE demonstrated greater dilution of CFSE post-treatment with 200 ng/mL IGF-1, indicative of enhanced proliferation of Treg populations (FIG. 3B).

FIG. 4A and FIG. 4B are a set of graphs demonstrating the effect of treatment with istiratumab on the mean percent expression of IGF-1R+ cells (per total lymphocyte population) from mouse splenocyte-derived Treg (defined as CD4+CD25+FoxP3+) and CD8+ cells (FIG. 4A) and the mean percent expression of ErbB3+ cells (per total lymphocyte population) from mouse splenocyte-derived Tregs (FIG. 4B), as measured by quantitative flow cytometry.

FIG. 5 is a graph demonstrating the effect of treatment of IGF-1 and istiratumab, alone and in combination, on splenocyte-derived FoxP3+ cells per CD4+CD25+ cells (represented as % Tregs) in vitro, as measured by flow cytometry.

FIG. 6 is a graph demonstrating the effects of treatment with phosphate-buffered saline (PBS, representing the vehicle control treatment) or istiratumab on A20 B cell lymphoma tumors inoculated subcutaneously into immuno-competent (wild-type, wt) and athymic (nu/nu) mice. The x-axis is days post tumor cell implantation and the y-axis is tumor volume (mm³).

FIG. 7 is a graph demonstrating the effects of treatment with PBS or istiratumab on MC38 colorectal tumors inoculated subcutaneously into immuno-competent (wild-type, wt) and athymic (nu/nu) mice. The x-axis is days post tumor cell implantation and the y-axis is mean tumor volume (mm³).

FIG. 8 is a graph demonstrating the effects of treatment with PBS or istiratumab on B16-F10 melanoma tumors inoculated subcutaneously into immuno-competent (wild-type, wt) and athymic (nu/nu) mice. The x-axis is days post tumor cell implantation and the y-axis is mean tumor volume (mm³).

FIG. 9A, FIG. 9B and FIG. 9C is a series of graphs demonstrating the basal and phosphorylated expression of IGF-1R, ErbB3, phosphorylated AKT (pAKT), phosphorylated ERK1/2 (pERK1/2) and beta actin in A20 B cell lymphoma cells (ATCC® TIB-208) by western blotting (A) and the effects of treatment with PBS, istiratumab, anti-PD-L1 (atezolizumab) and istiratumab+anti-PD-L1 in combination on mice inoculated with IGF-1R/ErbB3 negative A20 tumor cells (B, in which each x-axis is days post-tumor cell implantation and each y-axis is tumor volume (mm³)). In part C, mice who achieved a complete response to treatment (CR; n=4, data points plotted using triangles) with anti-PD-L1+istiratumab (as outlined in part B) retain anti-tumor immunity upon re-challenge with A20 cells two months after stopping drug treatment. Data from naïve mice inoculated with A20 cells (to confirm tumorigenicity of the inoculated cells) are plotted using diamonds.

FIG. 10A and FIG. 10B is a set of data demonstrating the basal expression of IGF-1R, ErbB3, pAKT, pERK1/2 and beta actin in WEHI 164 fibrosarcoma cells (ATCC® CRL-1751™) by western blotting (A). Panel B shows the effects of treatment with PBS, istiratumab, anti-PD-L1 (atezolizumab) and istiratumab+anti-PD-L1 in combination on mice inoculated with IGF-1R/ErbB3 positive WEHI 164 tumor cells. The x-axis is days post tumor cell implantation and the y-axis is mean tumor volume (mm³).

FIG. 11A and FIG. 11B is a series of graphs demonstrating the basal expression of IGF-1R, ErbB3, pAKT, pERK1/2 and beta actin in MC38 colorectal cells by western blotting (A). FIG. 11B shows the effects of treatment with PBS, istiratumab, anti-PD-L1 (atezolizumab) and istiratumab+anti-PD-L1 in combination on mice inoculated with IGF-1R/ErbB3 positive MC38 tumor cells in which each x-axis is days post tumor cell implantation and each y-axis is tumor volume (mm³).

FIG. 12A and FIG. 12B is a set of data demonstrating the basal expression of IGF-1R, ErbB3, pAKT, pERK1/2 and beta actin in B16-F10 melanoma cells by western blotting (A). Panel B shows the effects of treatment with PBS, istiratumab, anti-CTLA-4 (clone 9D9) and istiratumab+anti-CTLA-4 in combination on mice inoculated with IGF-1R/ErbB3 positive B16-F10 tumor cells in which each x-axis is days post tumor cell implantation and each y-axis is tumor volume (mm³).

FIG. 13 is a set of data showing the effects of treatment with PBS, istiratumab, anti-PD-1 (clone J43) and istiratumab+anti-PD-1 in combination on mice inoculated with IGF-1R/ErbB3 positive B16-F10 tumor cells in which each x-axis is days post tumor cell implantation and each y-axis is tumor volume (mm³).

FIG. 14A and FIG. 14B is a set of data showing the expression of IGF-1R and ErbB3 on various human whole blood-derived immune cell populations.

FIG. 15 is a set of data demonstrating the effects of the ligands heregulin (HRG) or IGF-1, and of MM-141 on markers of plasmacytoid dendritic cell (pDC) activity (CD83, CD86; measured by mean fluorescence intensity), interferon alpha (IFNα) expression (pg/mL media) and PD-L1 expression (measured by mean fluorescence intensity) post-activation with SD-101, as measured by flow cytometry.

FIG. 16 is a set of histograms showing the effect of MM-141 on the activity of myeloid-derived suppressor cells (MDSCs) on unactivated and activated CD4+ T cell proliferation, as measured by CFSE-FITC staining analyzed by flow cytometry.

FIG. 17A is a list of antagonistic anti-receptor antibodies.

FIG. 17B is a list of agonistic anti-receptor antibodies.

FIG. 17C is a list of anti-ligand antibodies.

### DETAILED DESCRIPTION

The current disclosure provides compositions, methods, and kits for the treatment of disorders in human and non-human patients in need thereof. Specifically, this disclosure provides methods of treatment for cancer in a patient using bispecific anti-IGF-1R, anti-ErbB3 antibodies in combination with one or more therapeutic agents that modulate immune suppressor cell, e.g., regulatory T-cell, activity (which may include but are not limited to: a small molecule immunomodulatory agent, an immunomodulatory antibody that does not bind human IGF1, an anticancer vaccine, and a radiation dose) or as a monotherapy or a combination therapy. An exemplary antibody is istiratumab (also described herein as P4-G1-M1.3). Istiratumab is demonstrated herein to be effective against cancer types expressing IGF-1R and ErbB3 on the surface of the cancer cell both with and without the addition of one or more therapeutics agents that impede regulatory T-cell activity. The combination therapy of the bispecific antibody and an agent that impedes regulatory T-cell activity has an effect on treating cancer cells that is greater than when either agent is administered at the same dose as a monotherapy. Further, istiratumab is demonstrated herein to be effective even against cancer types that do not express IGF-1R and ErbB3 on the surface of the cancer cell (see Example 4, utilizing the A20 cell line), and its anti-cancer activity primarily arises through recruitment of the antitumor activity of T cells.

### Definitions

For convenience, the meaning of certain terms and phrases used in the specification, examples, and appended claims, are provided below.

“Agent,” refers to an active molecule, e.g., a therapeutic protein, e.g., a drug.

“Aa substitution” refers to the replacement of one specific amino acid (“aa”) in a protein with another aa. A substitution may be a conservative substitution, as defined below.

“Administer” or “administration” refers to the act of injecting or otherwise physically delivering a substance as it exists outside the body (e.g., a formulation of the molecules disclosed herein) into a patient, such as by mucosal, intradermal, intravenous, intramuscular delivery and/or any other method of physical delivery described herein or known in the art. When a disease, or a symptom thereof, is being treated, administration of the substance typically occurs after the onset of the disease or symptoms thereof. When diseases, or symptoms thereof, are being prevented, administration of the substance typically occurs before the onset of the disease or symptoms thereof.

“Anti-ErbB3 binding site” refers to a binding site that binds specifically to human ErbB3.

“Anti-IGF-1R binding site” refers to a binding site that binds specifically to human IGF-1R.

“Antigen binding site” refers to a binding site that comprises the VH and/or VL domain of an antibody, or at least one CDR thereof. For example, an antigen binding site may comprise, consist essentially of, or consist of a VHCDR3 alone or together with a VHCDR2 and optionally a VHCDR1. In certain embodiments, an antigen binding site comprises a VH domain and a VL domain, which may be present on the same polypeptide, or on two different polypeptides, e.g., the VH domain is present on a heavy chain and a VL domain is present on a light chain.

“Antigen-binding portion” of an antibody refers to one or more fragments of an antibody that retain the ability to specifically bind to an antigen (e.g., IGF-1R or ErbB3). It has been shown that the antigen-binding function of an antibody can be retained by fragments of a full-length antibody. Examples of binding fragments encompassed within the term “antigen-binding portion” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) an Fd fragment consisting of the VH and CH1 domains; (iv) an Fv fragment consisting of the VL and VH domains of a single arm of an antibody, (v) a dAb fragment which consists of a VH domain; and (vi) an isolated complementarity determining region (CDR). Furthermore, although VL and VH are two domains of an Fv fragment, VL and VH are coded for by separate genes, they can be joined, using recombinant methods, by a synthetic linker that enables them to be made as a single protein chain in which the VL and VH regions pair to form monovalent proteins, known as single chain Fvs (scFvs) see U.S. Pat. No. 5,892,019. Such single chain antibodies are also intended to be encompassed within the term “antigen-binding portion” of an antibody. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites.

“Binding affinity” refers to the strength of a binding interaction and includes both the actual binding affinity as well as the apparent binding affinity. The actual binding affinity is a ratio of the association rate over the disassociation rate. The apparent affinity can include, for example, the avidity resulting from a polyvalent interaction. Dissociation constant (Kd), is typically the reciprocal of the binding affinity, and may be conveniently measured using a surface plasmon resonance assay (e.g., as determined in a BIACORE 3000 instrument (GE Healthcare) e.g., using recombinant ErbB3 as the analyte and an anti-ErbB3 antibody as the ligand) or a cell binding assay, each of which assays is described in Example 3 of U.S. Pat. No. 7,846,440.

“Binding moiety,” “binding domain,” or “binding site,” refers to the portion, region, or site of a binding polypeptide or, when so specified, of a heavy or light chain thereof, that is directly involved in mediating the specific binding of an antibody to a target molecule (i.e., an antigen). Exemplary binding domains include an antigen binding site, a receptor binding domain of a ligand, a ligand binding domain of a receptor or an enzymatic domain. In preferred embodiments, the binding domain comprises or consists of an antigen binding site (e.g., comprising a variable heavy (VH) chain sequence and variable light (VL) chain sequence or six CDRs from an antibody placed into alternative framework regions (e.g., human framework regions optionally comprising one or more aa substitutions). In certain embodiments, a binding site may be comprised essentially only of a VH or a VL chain sequence. A binding site may be entirely from one species, e.g., it has only sequences that derive from the germline sequences of one species. For example, a binding site may be human (i.e., from the human species), mouse, or rat. A binding site may also be humanized, i.e., the CDRs are from one species and the frameworks (FRs) are from another species. For example, a binding site may have CDRs that were derived from a mouse antibody and FRs that are from the human species. Certain humanized binding sites comprise mutations in one or more CDR to make the CDRs look more like the CDRs of the donor antibody. Certain humanized antibodies may also comprise mutations in one or more FR. Generally mutations in a binding site may enhance the affinity of binding of the binding site to its target antigen, and/or they may stabilize the binding site, e.g., to extend its half-life.

“CDR” or “complementarity determining region” refers to the noncontiguous antigen combining sites found within the VR of both heavy and light chain polypeptides. These particular regions have been described by Kabat et al., J. Biol. Chem. 252, 6609-6616 (1977) and Kabat et al., Sequences of protein of immunological interest. (1991), and by Chothia et al., J. Mol. Biol. 196:901-917 (1987) and by MacCallum et al., J. Mol. Biol. 262:732-745 (1996), where the definitions include overlapping or subsets of aa residues when compared against each other. The aa residues which encompass the CDRs as defined by each of the above cited references are set forth for comparison. As used herein, and if not otherwise specified, “CDR” is as defined by Kabat.

TABLE 1
CDR definitions

| | CDR Definitions | | |
|---|---|---|---|
| | Kabat¹ | Chothia² | MacCallum³ |
| VHCDR1 | 31-35 | 26-32 | 30-35 |
| VHCDR2 | 50-65 | 53-55 | 47-58 |
| VHCDR3 | 95-102 | 96-101 | 93-101 |
| VLCDR1 | 24-34 | 26-32 | 30-36 |
| VLCDR2 | 50-56 | 50-52 | 46-55 |
| VLCDR3 | 89-97 | 91-96 | 89-96 |

¹Residue numbering follows the nomenclature of Kabat et al., 1991, supra
²Residue numbering follows the nomenclature of Chothia et al., supra
³Residue numbering follows the nomenclature of MacCallum et al., supra

“CH1 domain” refers to the heavy chain immunoglobulin constant domain located between the VH domain and the hinge. It spans EU positions 118-215. A CH1 domain may be a naturally occurring CH1 domain, or a naturally occurring CH1 domain in which one or more aas have been substituted, added or deleted, provided that the CH1 domain has the desired biological properties. A desired biological activity may be a natural biological activity, an enhanced biological activity or a reduced biological activity relative to the naturally occurring sequence.

“CH2 domain” refers to the heavy chain immunoglobulin constant domain that is located between the hinge and the CH3 domain. It spans EU positions 231-340. A CH2 domain may be a naturally occurring CH2 domain, or a naturally occurring CH2 domain in which one or more aas have been substituted, added or deleted, provided that the CH2 domain has the desired biological properties. A desired biological activity may be a natural biological activity, an enhanced biological activity or a reduced biological activity relative to the naturally occurring sequence.

“CH3 domain” refers to the heavy chain immunoglobulin constant domain that is located C-terminally of the CH2 domain and spans approximately 110 residues from the N-terminus of the CH2 domain, e.g., about positions 341-446b (EU numbering system). A CH3 domain may be a naturally occurring CH3 domain, or a naturally occurring CH3 domain in which one or more aas (“aas”) have been substituted, added or deleted, provided that the CH3 domain has the desired biological properties. A desired biological activity may be a natural biological activity, an enhanced biological activity or a reduced biological activity relative to the naturally occurring sequence. A CH3 domain may or may not comprise a C-terminal lysine.

“CH4 domain” refers to the heavy chain immunoglobulin constant domain that is located C-terminally of the CH3 domain in IgM and IgE antibodies. A CH4 domain may be a naturally occurring CH4 domain, or a naturally occurring CH4 domain in which one or more aas have been substituted, added or deleted, provided that the CH4 domain has the desired biological properties. A desired biological activity may be a natural biological activity, an enhanced biological activity or a reduced biological activity relative to the naturally occurring sequence.

“CL domain” refers to the light chain immunoglobulin constant domain that is located C-terminally to the VH domain. It spans about Kabat positions 107A-216. A CL domain may be a naturally occurring CL domain, or a naturally occurring CL domain in which one or more aas have been substituted, added or deleted, provided that the CL domain has the desired biological properties. A desired biological activity may be a natural biological activity, an enhanced biological activity or a reduced biological activity relative to the naturally occurring sequence. A CL domain may or may not comprise a C-terminal lysine.

“Combination therapy,” “co-administration,” “co-administered” or “concurrent administration” (or minor variations of these terms) refers to simultaneous administration of at least two therapeutic agents to a patient or their sequential administration within a time period during which the first administered therapeutic agent is still present in the patient (e.g., in the patient's plasma or serum) when the second administered therapeutic agent is administered.

“Conservative substitution” or “conservative aa substitution” refers to the replacement of one or more aa residues in a protein or a peptide with, for each particular pre-substitution aa residue, a specific replacement aa that is known to be unlikely to alter either the confirmation or the function of a protein or peptide in which such a particular aa residue is substituted for by such a specific replacement aa. Such conservative substitutions typically involve replacing one aa with another that is similar in charge and/or size to the first aa, and include replacing any of isoleucine (I), valine (V), or leucine (L) for each other, substituting aspartic acid (D) for glutamic acid (E) and vice versa; glutamine (Q) for asparagine (N) and vice versa; and serine (S) for threonine (T) and vice versa. Other substitutions are known in the art to be conservative in particular sequence or structural environments. For example, glycine (G) and alanine (A) can frequently be substituted for each other to yield a conservative substitution, as can be alanine and valine (V). Methionine (M), which is relatively hydrophobic, can frequently conservatively substitute for or be conservatively substituted by leucine or isoleucine, and sometimes valine. Lysine (K) and arginine (R) are frequently interchangeable in locations in which the significant feature of the aa residue is its charge and the differing pK's of these two basic aa residues are not expected to be significant. The effects of such substitutions can be calculated using substitution score matrices such PAM120, PAM-200, and PAM-250. Other such conservative substitutions, for example, substitutions of entire regions having similar hydrophobicity characteristics (e.g., transmembrane domains), are well known.

A CR domain on a light chain of an immunoglobulin is referred to interchangeably as a “CL,” “light chain CR domain,” “CL region” or “CL domain.” A constant domain on a heavy chain (e.g., hinge, CH1, CH2 or CH3 domains) of an immunoglobulin is referred to interchangeably as a “CH,” “heavy chain constant domain,” “CH” region or “CH domain.” A variable domain on an immunoglobulin light chain is referred to interchangeably as a “VL,” “light chain variable domain,” “VL region” or “VL domain.” A variable domain on an immunoglobulin heavy chain is referred to interchangeably as a “VH,” “heavy chain variable domain,” “VH region” or “VH domain.”

“Domain” refers to a region, e.g., an independently folding, globular region or a non-globular region (e.g., a linker domain), of a heavy or light chain polypeptide which may comprise peptide loops (e.g., 1 to 4 peptide loops) that may be stabilized, for example, by a f-pleated sheet and/or an intrachain disulfide bond. The constant and VRs of immunoglobulin heavy and light chains are typically folded into domains. In particular, each one of the CH1, CH2, CH3, CH4, CL, VH and VL domains typically form a loop structure.

“Dosage” refers to parameters for administering a drug in defined quantities per unit time (e.g., per hour, per day, per week, per month, etc.) to a patient. Such parameters include, e.g., the size of each dose. Such parameters also include the configuration of each dose, which may be administered as one or more units, e.g., as one or more administrations, e.g., either or both of orally (e.g., as one, two, three or more pills, capsules, etc.) or injected (e.g., as a bolus or infusion). Dosage sizes may also relate to doses that are administered continuously (e.g., as an intravenous infusion over a period of minutes or hours). Such parameters further include frequency of administration of separate doses, which frequency may change over time.

“Dose” refers to an amount of a drug given in a single administration.

“EC₅₀” or “EC50” refers to the concentration of a molecule, e.g., a PBA that provides 50% of the maximal effect of the protein on a particular system such as a binding assay or a signal transduction pathway.

“Effective amount,” “effective amounts,” effective dosage,” “effective dosages,” “effective doses,” or “effective dose” refers to an amount (administered in one or more doses) of an antibody, protein or additional therapeutic agent, which amount is sufficient to provide effective treatment to a subject in need thereof. “Effective treatment” refers to a decrease or cessation in the symptoms of a disorder in the subject. In the case of cancer, effective treatment may comprise, but is not limited to, one or more of the following results: reduction in size of one or more tumors, reduction in number of tumors in a subject, reduction in number of cancerous cells in a subject, inhibition of tumor growth in a subject, or prolongation of survival time for an animal with cancer. An effective amount of the compositions of the present disclosure, for the treatment of the described conditions vary depending upon many different factors, including (but not limited to) means of administration, target site, physiological state of the patient, whether the patient is human or an animal, and other medications administered. Treatment dosages may be titrated using routine methods known to those of skill in the art to optimize safety and efficacy.

“ErbB3” and “HER3” refer to ErbB3 protein, as described herein and in U.S. Pat. No. 5,480,968. The human ErbB3 protein sequence is provided herein as SEQ ID NO: 444, which corresponds to SEQ ID NO:4 of U.S. Pat. No. 5,480,968, wherein the first 19 aas correspond to the leader sequence that is cleaved from the mature protein. ErbB3 is a member of the ErbB family of receptors, other members of which include ErbB1 (EGFR), ErbB2 (HER2/Neu) and ErbB4. While ErbB3 itself lacks tyrosine kinase activity, but is itself phorphorylated upon dimerization of ErbB3 with another ErbB family receptor, e.g., ErbB1, ErbB2 and ErbB4, which are receptor tyrosine kinases. Ligands for the ErbB family include heregulin (HRG), betacellulin (BTC), epidermal growth factor (EGF), heparin-binding epidermal growth factor (HB-EGF), transforming growth factor alpha (TGF-α), amphiregulin (AR), epigen (EPG) and epiregulin (EPR). The aa sequence of human ErbB3 is provided at Genbank Accession No. NP_001973.2 (receptor tyrosine-protein kinase erbB-3 isoform 1 precursor) and is assigned Gene ID: 2065.

“EU” indicates that aa positions in a heavy chain CR, including aa positions in the CH1, hinge, CH2, and CH3 domains, are numbered herein according to the EU index numbering system (see Kabat et al., in “Sequences of Proteins of Immunological Interest”, U.S. Dept. Health and Human Services, 5th edition, 1991).

“Fab” refers to the antigen binding portion of an antibody, comprising two chains: a first chain that comprises a VH domain and a CH1 domain and a second chain that comprises a VL domain and a CL domain. Although a Fab is typically described as the N-terminal fragment of an antibody that was treated with papain and comprises a portion of the hinge region, it is also used herein as referring to a binding domain wherein the heavy chain does not comprise a portion of the hinge.

“Fc region” refers to the portion of a single immunoglobulin heavy chain beginning in the hinge region just upstream of the papain cleavage site (i.e. residue 216 in IgG, taking the first residue of heavy chain CR to be 114) and ending at the C-terminus of the antibody. Accordingly, a complete Fc region comprises at least a hinge, a CH2 domain, and a CH3 domain. Two Fc regions that are dimerized are referred to as “Fc” or “Fc dimer.” An Fc region may be a naturally occurring Fc region, or a naturally occurring Fc region in which one or more aas have been substituted, added or deleted, provided that the Fc region has the desired biological properties. A desired biological activity may be a natural biological activity, an enhanced biological activity or a reduced biological activity relative to the naturally occurring sequence.

“Formulation” and “composition” refer to a product containing the specified ingredients (e.g., an anti-IGF-1R, anti-ErbB3 bispecific antibody) in, optionally, the specified amounts, as well as any product which results, directly or indirectly, from the combination of the specified ingredients in, optionally, the specified amounts.

“Excipient” or “Excipients” refers to inert substances that are commonly used as a diluent, vehicle, preservative, binder, stabilizing agent, etc. for drugs and includes, but is not limited to, proteins (e.g., serum albumin, etc.), amino acids (e.g., aspartic acid, glutamic acid, lysine, arginine, glycine, histidine, etc.), fatty acids and phospholipids (e.g., alkyl sulfonates, caprylate, etc.), surfactants (e.g., SDS, polysorbate, nonionic surfactant, etc.), saccharides (e.g., sucrose, maltose, trehalose, etc.) and polyols (e.g., mannitol, sorbitol, etc.). See, also, Remington's Pharmaceutical Sciences (1990) Mack Publishing Co., Easton, Pa., which is hereby incorporated by reference in its entirety.

“Framework region” or “FR” or “FR region” includes the aa residues that are part of the VR, but are not part of the CDRs (e.g., using the Kabat definition of CDRs). Therefore, a VR framework is between about 100-120 aas in length but includes only those aas outside of the CDRs. For the specific example of a heavy chain VR and for the CDRs as defined by Kabat et al., 1991, ibid., framework region 1 corresponds to the domain of the VR encompassing aas 1-30; framework region 2 corresponds to the domain of the VR encompassing aas 36-49; framework region 3 corresponds to the domain of the VR encompassing aas 66-94, and framework region 4 corresponds to the domain of the VR from aas 103 to the end of the VR. The framework regions for the light chain are similarly separated by each of the light chain VR CDRs. Similarly, using the definition of CDRs by Chothia et al. or McCallum et al. the framework region boundaries are separated by the respective CDR termini as described above. In preferred embodiments, the CDRs are as defined by Kabat.

“Full-length antibody” is an antibody that comprises one or more heavy chains and one or more light chains. Each heavy chain is comprised of a heavy chain VR (abbreviated herein as VH) and a heavy chain CR. The heavy chain CR is comprised of three domains CH1, CH2, and CH3, and optionally a fourth domain, CH4. Each light chain is comprised of a light chain VR (abbreviated herein as VL) and a light chain CR. The light chain CR is comprised of one domain, CL. The VH and VL regions can be further subdivided into regions of hypervariability, termed complementarity determining regions (CDR), interspersed with regions that are more conserved, termed framework regions (FR). Each VH and VL is typically composed of three CDRs and four FRs, arranged from amino-terminus to carboxy-terminus in the following order: FR1, CDR1, FR2, CDR2, FR3, CDR3, and FR4. Immunoglobulin proteins can be of any type class (e.g., IgG, IgE, IgM, IgD, IgA and IgY) or, subclass (e.g., IgG 1, IgG2, IgG 3, IgG4, IgA1 and IgA2) or subclass.

“Gly-Ser linker” or “Gly-Ser peptide” refers to a peptide that consists of glycine and serine residues. An exemplary Gly-Ser peptide comprises the aa sequence (Gly₄Ser)n (SEQ ID NO:395), wherein n=1, 2, 3, 4, 5, 6, 7, 8, 9, 10, 11, 12, 13, 14, 15, 16, 17, 18, 19, 20 or more. In certain embodiments, n is a number between 1 and 5, n is a number between 6 and 10, n is a number between 11 and 15, n is a number between 16 and 20, n is a number between 21 and 25, or n is a number between 26 and 30.

“Heavy chain immunoglobulin CR” or “HC Ig CR” may comprise a CH1 domain and an Fc region, which Fc region may comprise a hinge, a CH2 domain, a CH3 domain and/or a CH4 domain. A light chain immunoglobulin CR may comprise a CL domain.

“Hinge” or “hinge region” or “hinge domain” refers to the flexible portion of a heavy chain located between the CH1 domain and the CH2 domain. It is approximately 25 aas long, and is divided into an “upper hinge,” a “middle hinge,” and a “lower hinge.” A hinge may be a naturally occurring hinge, or a naturally occurring hinge in which one or more aas have been substituted, added or deleted, provided that the hinge has the desired biological properties. A desired biological activity may be a natural biological activity, an enhanced biological activity or a reduced biological activity relative to the naturally occurring sequence.

“IC₅₀,” or “IC50” refers to the concentration of a molecule, e.g., a PBA, that provides a 50% inhibition of a maximal activity (e.g., a response to a stimulus or a constitutive activity), i.e., a concentration that reduces the activity to a level halfway between the maximal activity and the baseline. The IC₅₀ value may be converted to an absolute inhibition constant (Ki) using, e.g., the Cheng-Prusoff equation. In a system that is inhibited by a binding agent, such as an antibody or a bispecific binding protein provided herein, the IC₅₀ may be indistinguishable from the EC₅₀.

“IGF-1R” or “IGF1R” refers to the receptor for insulin-like growth factor 1 (IGF-1, formerly known as somatomedin C). IGF-1R also binds to, and is activated by, insulin-like growth factor 2 (IGF-2). IGF1-R is a receptor tyrosine kinase, which upon activation by IGF-1 or IGF-2 is auto-phosphorylated. The aa sequence of human IGF-1R precursor is provided at Genbank Accession No. NP_000866 and is assigned Gene ID: 3480.

“IgG-(scFv)₂” indicates a tetravalent PBA consisting of an IgG having two N-terminal Fab binding sites each comprised of an IgG heavy chain and an IgG light chain, wherein the C-terminus of each heavy chain is linked to an scFv having a binding site comprised of a VH domain and a VL domain. When the immunoglobulin CRs are those of an IgG1, the PBA is referred to as an “IgG1-(scFv)₂.” Exemplary IgG1-(scFv)₂ PBAs are those where the four binding sites comprise two essentially identical anti-IGF-1R binding sites and two essentially identical anti-ErbB3 binding sites. Certain tetravalent PBAs described below and in U.S. Pat. No. 8,476,409 each comprise two joined essentially identical subunits, each subunit comprising a heavy and a light chain that are disulfide bonded to each other, e.g., M7-G1-M78 (SEQ ID NO:284 and SEQ ID NO:262), P4-G1-M1.3 (SEQ ID NO:226 and SEQ ID NO:204), and P4-G1-C8 (SEQ ID NO:222 and SEQ ID NO:204), are exemplary embodiments of such IgG1-(scFv)₂ proteins.

“Immunoglobulin CR” or “Ig CR” refers to the parts of an immunoglobulin, (i.e., an antibody,) outside of its variable domains. In certain embodiments, an immunoglobulin CR comprises a “heavy chain immunoglobulin CR” and a “light chain immunoglobulin CR.”

“Inhibition” of a biological activity by a binding protein refers to any reproducibly detectable decrease in biological activity mediated by the binding protein. In some embodiments, inhibition provides a statistically significant decrease in biological activity, e.g., a decrease of about 5%, 10%, 20%, 30%, 40%, 50%, 60%, 70%, 80%, 90%, or 100% in biological activity relative to the biological activity determined in the absence of the binding protein.

“Isolated,” in reference to polynucleotides, polypeptides or proteins, means that the polynucleotide, polypeptide or protein is substantially removed from polynucleotides, polypeptides, proteins or other macromolecules with which it, or its analogues, occurs in nature. Although the term “isolated” is not intended to require a specific degree of purity, typically, the protein will be at least about 75% pure, more preferably at least about 80% pure, more preferably at least about 85% pure, more preferably at least about 90% pure, more preferably still at least about 95% pure, and most preferably at least about 99% pure.

“Kabat” in conjunction with designation of immunoglobulin aa sequence positions indicates that aa positions in a light chain CR (e.g., CL domain) are numbered according to the Kabat index numbering system (see Kabat et al., 1991., op. cit.).

“Linked to” refers to direct or indirect linkage or connection of, in context, aas or nucleotides. An “indirect linkage” refers to a linkage that is mediated through a linker or a domain, comprising, e.g., one or more aas or nucleotides. A “direct linkage” or “linked directly” when referring to two polypeptide segments refers to the presence of covalent bond between the two polypeptide segments, e.g., the two polypeptide segments are joined contiguously without intervening sequences.

“Linker” refers to one or more aas connecting two domains or regions together. A linker may be flexible to allow the domains being connected by the linker to form a proper three dimensional structure thereby allowing them to have the required biological activity. A linker connecting the VH and the VL of an scFv is referred to herein as an “scFv linker.” A linker connecting the N-terminus of a VH domain or the C-terminus of the CH3 domain to a second VH domain, e.g., that of an scFv is referred to as a “connecting linker.”

“Module” refers to a structurally and/or functionally distinct part of a PBA, such a binding site (e.g., an scFv domain or a Fab domain) and the Ig constant domain. Modules provided herein can be rearranged (by recombining sequences encoding them, either by recombining nucleic acids or by complete or fractional de novo synthesis of new polynucleotides) in numerous combinations with other modules to produce a wide variety of PBAs, e.g., as disclosed herein. For example, an “SF” module refers to the binding site “SF,” i.e., comprising at least the CDRs of the SF VH and SF VL domains. A “C8” module refers to the binding site “C8.”

“Patient,” “patients,” “subject, or “subjects” refers to one or more human or non-human animals having one or more conditions treatable by the compositions described in the instant application. In some embodiments, the patient may have one or more types of cancer.

“PBA” refers to a polyvalent bispecific antibody, an artificial hybrid protein comprising at least two different binding moieties or domains and thus at least two different binding sites (e.g., two different antibody binding sites), wherein one or more of the pluralities of the binding sites are covalently linked, e.g., via peptide bonds, to each other. One PBA described herein is an anti-IGF-1R+anti-ErbB3 PBA, which is a polyvalent bispecific antibody that comprises one or more first binding sites binding specifically to an IGF-1R protein, e.g., a human IGF-1R protein, and one or more second binding sites binding specifically to an ErbB3 protein, e.g., a human ErbB3 protein. An anti-IGF-1R+anti-ErbB3 PBA is so named regardless of the relative orientations of the anti-IGF-1R and anti-ErbB3 binding sites in the molecule, whereas when the PBA name comprises two antigens separated by a slash (/) the antigen to the left of the slash is amino terminal to the antigen tot the right of the slash. A PBA may be a bivalent binding protein, a trivalent binding protein, a tetravalent binding protein or a binding protein with more than 4 binding sites. An exemplary PBA is a tetravalent bispecific antibody, i.e., an antibody that has 4 binding sites, but binds to only two different antigens or epitopes. Exemplary bispecific antibodies are tetravalent “anti-IGF-1R/anti-ErbB3” PBAs and “anti-ErbB3/anti-IGF-1R” PBAs. Typically the N-terminal binding sites of a tetravalent PBA are Fabs and the C-terminal binding sites are scFvs.

“Percent identical” or “% identical” refers to two or more nucleic acid or polypeptide sequences or subsequences that are the same (100% identical) or have a specified percentage of nucleotide or aa residues that are the same, when the two sequences are aligned for maximum correspondence and compared. To align for maximum correspondence, gaps may be introduced into one of the sequences being compared. The aa residues or nucleotides at corresponding positions are then compared and quantified. When a position in the first sequence is occupied by the same residue as the corresponding position in the second sequence, then the sequences are identical at that position. The percent identity between the two sequences is a function of the number of identical positions shared by the sequences (e.g., % identity=# of identical positions/total # of positions (e.g., overlapping positions)×100). In certain embodiments, the two sequences are the same length. The determination that one sequence is a measured % identical with another sequence can be determined using a mathematical algorithm. A non-limiting example of a mathematical algorithm utilized for such comparison of two sequences is incorporated in the ALIGN program (version 2.0) which is part of the GCG sequence alignment software package. When utilizing the ALIGN program e.g., for comparing aa sequences, a PAM120 weight residue table, a gap length penalty of 12, and a gap penalty of 4 may be used. Additional algorithms for sequence analysis are well known in the art and many are available online.

“Pharmaceutical combination” or “pharmaceutical composition” refers to a product that results from the mixing or combining of more than one active ingredient and includes both fixed and non-fixed combinations of the active ingredients. The term “fixed combination” means that the active ingredients, e.g., a compound useful in the disclosed methods and a co-agent, are both administered to a patient simultaneously in the form of a single entity or dosage. The term “non-fixed combination” means that the active ingredients, e.g., a compound useful in the disclosed methods and a co-agent, are both administered to a patient as separate entities either simultaneously, concurrently or sequentially with no specific time limits, wherein such administration provides therapeutically effective levels of the two compounds in the body of the patient. The latter also applies to cocktail therapy, e.g., the administration of three or more active ingredients.

“Portion” or “fragment” (e.g., of a domain) of a reference moiety refers to a discrete part of the whole reference moiety (e.g., domain, e.g., a naturally occurring domain) that is at least, or at most 10% 20%, 30%, 40%, 50%, 60%, 70%, 80%, 90%, 95%, 98%, or 99% of the size of the reference moiety.

“Preferentially binds” or “preferentially binding” refers to the specific binding of one binding agent to a target epitope or target epitopes as stronger than the specific binding of a second binding agent to the same target epitope or target epitopes.

“scFv linker” refers to a peptide or polypeptide domain interposed between the VL and VH domains of an scFv. scFv linkers preferably allow orientation of the VL and VH domains in a antigen binding conformation. In one embodiment, an scFv linker comprises or consists of a peptide or polypeptide linker that only comprises glycines and serines (a “Gly-Ser linker”). In certain embodiments, an scFv linker comprises a disulfide bond.

“scFv protein” refers to a binding protein that consists of a single polypeptide comprising one light chain variable domain (VL), and one heavy chain variable domain (VH), wherein each variable domain is derived from the same or different antibodies. scFv proteins typically comprise an scFv linker interposed between the VH domain and the VL domain. ScFv proteins are known in the art and are described, e.g., in U.S. Pat. No. 5,892,019.

“Similarity” or “percent similarity” in the context of two or more polypeptide sequences, refer to two or more sequences or subsequences that have a specified percentage of aa residues that are the same or conservatively substituted when compared and aligned for maximum correspondence. By way of example, a first aa sequence can be considered similar to a second aa sequence when the first aa sequence is at least 50%, 60%, 70%, 75%, 80%, 90%, or even 95% identical, or conservatively substituted, to the second aa sequence when compared to an equal number of aas as the number contained in the first sequence, or when compared to an alignment of polypeptides that has been aligned by a computer similarity program known in the art. These terms are also applicable to two or more polynucleotide sequences.

“Small molecule immunomodulator” is a small molecule agent that effects one or more functions of the immune system.

“Specific binding,” “specifically binds,” “selective binding,” and “selectively binds,” as well as “immunospecifically binds” “binds specifically” “binds selectively,” are terms that may be used when referring to the binding of a binding site to its target epitope or a combination of binding sites to their target epitopes, means that the binding site(s) exhibit(s) immunospecific binding to the target epitope(s).

A binding site that binds specifically to an epitope exhibits appreciable affinity for a target epitope and, generally, does not exhibit cross-reactivity with other epitopes in that it does not exhibit appreciable affinity to any unrelated epitope and preferably does not exhibit affinity for any unrelated epitope that is equal to, greater than, or within two orders of magnitude lower than the affinity for the target epitope. “Appreciable” or preferred binding includes binding with a dissociation constant (Kd) of 10⁻⁸, 10⁻⁹ M, 10⁻¹⁰, 10⁻¹¹, 10⁻¹² M, 10⁻¹³ M or an even lower Kd value. Note that lower values for Kd (dissociation constant) indicate higher binding affinity, thus a Kd of 10⁻⁷ is a higher Kd value than a Kd of 10⁻⁸, but indicates a lower binding affinity than a Kd of 10⁻⁸). Dissociation constants with values of about 10⁻⁷ M, and even as low as about 10⁻⁸ M, are at the high end of dissociation constants suitable for therapeutic antibodies. Binding affinities may be indicated by a range of dissociation constants, for example, 10⁻⁶ to 10⁻¹² M, 10⁻⁷ to 10⁻¹² M, 10⁻⁸ to 10⁻¹² M or better (i.e., or lower value dissociation constant). Dissociation constants in the nanomolar (10⁻⁹ M) to picomolar (10⁻¹² M) range or lower are typically most useful for therapeutic antibodies. Suitable dissociation constants are Kds of 50 nM or less (i.e., a binding affinity of 50 nM or higher—e.g., a Kd of 45 nM) or Kds of 40 nM, 30 nM, 20 nM, 10 nM, 1 nm, 100 pM, 10 pM or 1 pM or less. Specific or selective binding can be determined according to any art-recognized means for determining such binding, including, for example, according to Scatchard analysis and/or competitive binding assays.

Methods of Treatment

In one aspect, provided herein are methods of treating cancer (e.g., treating a patient having a solid tumor) comprising administering a therapeutically effective amount of an anti-IGF-1R and anti-ErbB3 antibody, preferably istiratumab (MM-141), alone or in combination with an immunomodulatory therapeutic agent.

In some embodiments, the immunomodulatory therapeutic agent is a human cytotoxic T-lymphocyte antigen 4 (CTLA-4)-blocking antibody. The immunomodulatory therapeutic agent can be indicated for the treatment of unresectable or metastatic melanoma. In some embodiments, cancer can be treated by administering istiratumab in combination with a CTLA-4 blocking antibody to a human patient diagnosed with cancer, such as cancer having a solid tumor. In some embodiments, istiratumab is administered alone or in combination with a CTLA-4 blocking antibody (e.g., ipilumumab) for the treatment of patients with unresectable or metastatic melanoma.

Preferably, the human patient does not have an auto-immune condition raising unacceptable risks of medically undesirable complications from the administration of istiratumab alone or in combination with the CTLA-4 blocking antibody. For example, patients having one or more of the following can be excluded from receiving a combination of istiratumab and the CTLA-4 blocking antibody: active autoimmune disease or those receiving systemic immunosuppression for organ transplantation. In some embodiments, patients can also be excluded from receiving the antineoplastic therapy who have a relevant medical history including adverse reactions or conditions such as myocarditis, angiopathy, temporal arteritis, vasculitis, polymyalgia rheumatica, conjunctivitis, blepharitis, episcleritis, scleritis, leukocytoclastic vasculitis, erythema multiforme, psoriasis, pancreatitis, arthritis, and autoimmune thyroiditis.

In some embodiments, the methods of treating cancer comprise the administration of an antineoplastic therapy, wherein the antineoplastic therapy consists of the administration of a therapeutically effective dose of istiratumab (e.g., 40 mg/kg or 2.8 grams fixed dose, or a reduced fixed dose according to the table below) once every two weeks, alone or in combination with the anti CTLA-4 antibody (e.g., 3 mg/kg ipilimumab as an intravenous infusion over 90 minutes, once every three weeks). In some embodiments, istiratumab is administered as an intravenous infusion, for example over a 90-120 minute time period (e.g., a 90-minute infusion once every two weeks or a 120-minute infusion once every two weeks, or a 120 minute infusion in the first dose, followed by a 90 minute infusion in subsequent doses). In some embodiments, the antineoplastic therapy includes or consists of one or more reductions in the dose of istiratumab as indicated in Table 2, administered alone or in combination with the PD-1 blocking antibody. In some embodiments, the antineoplastic therapy includes or consists of one or more reductions in the dose of istiratumab as indicated in Table 2, administered alone or in combination with the PD-L1 blocking antibody. In some embodiments, the antineoplastic therapy includes or consists of one or more reductions in the dose of istiratumab as indicated in Table 2, administered alone or in combination with the CTLA4 blocking antibody.

TABLE 2
Istiratumab Dose Schedule

| Dose Level | Dose | Frequency |
|---|---|---|
| Full dose | 2.8 g (10 vials) | Q2W |
| 1^{st} dose reduction | 2.24 g (8 vials) | Q2W |
| 2^{nd} dose reduction | 1.96 g (7 vials) | Q2W |
| If additional dose reductions are | Discontinue | Discontinue |
| required | | |

For example, in some embodiments, the method comprises administering an antineoplastic therapy to a human patient diagnosed with cancer, where the antineoplastic therapy consists of the administration of 2.8, 2.24 or 1.96 g of istiratumab to the patient (e.g., once every two weeks), where the human patient does not have a diagnosed active auto-immune disease. In another embodiment, the method comprises administering an antineoplastic therapy to a human patient diagnosed with cancer, where the antineoplastic therapy consists of the administration of 2.8, 2.24 or 1.96 g of istiratumab once every two weeks to the patient in combination with 3 mg/kg ipilimumab once every three weeks for a total of up to four doses of ipilumumab, where the human patient is diagnosed with unresectable or metastatic melanoma. Alternatively, in some embodiments, the antineoplastic therapy is administered once every week, and consists of the administration of istiratumab at a therapeutically effective dose of 6 mg/kg, 12 mg/kg or 20 mg/kg per dose.

In another aspect, provided herein are methods of treating cancer (e.g., treating a patient having a solid tumor) comprising the administration of a therapeutically effective amount of an anti-IGF-1R and anti-ErbB3 antibody, preferably istiratumab (MM-141), alone or in combination with a human programmed death receptor-1, or PD-1, blocking antibody. In some embodiments, the cancer can be treated by administration of istiratumab in combination with a PD-1 blocking antibody to a human patient (e.g., a patient diagnosed with a solid tumor). In some embodiments, istiratumab is administered alone or in combination with a PD-1 blocking antibody (e.g., nivolumab) for the treatment of patients with unresectable or metastatic melanoma and disease progression following ipilimumab and, if BRAF V600 mutation positive, a BRAF inhibitor.

In one embodiment, the human patient does not have an active auto-immune condition raising unacceptable risks of medically undesirable complications from the administration of istiratumab alone or in combination with the PD-1 blocking antibody. For example, in some embodiments, patients having one or more of the following can be excluded from receiving a combination of istiratumab and the PD-1 blocking antibody: patients with a diagnosed autoimmune disease, patients with prior ipilimumab-related Grade 4 adverse reactions (except for endocrinopathies) or Grade 3 ipilimumab-related adverse reactions that had not resolved or were inadequately controlled within 12 weeks of the initiating event, and patients with a condition requiring chronic systemic treatment with corticosteroids (>10 mg daily prednisone equivalent) or other immunosuppressive medications, a positive test for hepatitis B or C, and a history of HIV. In some embodiments, patients are excluded from therapy who have a medical history included immune-mediated adverse reactions such as pancreatitis, uveitis, demyelination, autoimmune neuropathy, adrenal insufficiency, and facial and abducens nerve paresis.

In some embodiments, antineoplastic therapy is administered once every two weeks, wherein the therapy consists of administering a therapeutically effective dose of istiratumab (e.g., 40 mg/kg or 2.8 grams fixed dose), alone or in combination with an anti PD-1 antibody (e.g., 3 mg/kg nivolumab as an intravenous infusion over 60 minutes). In some embodiments, istiratumab is administered as an intravenous infusion, for example over a 90-120 minute time period (e.g., a 90-minute infusion once every two weeks or a 120-minute infusion once every two weeks, or a 120 minute infusion in the first dose, followed by a 90 minute infusion in subsequent doses). In some embodiments, the antineoplastic therapy includes or consists of one or more reductions in the dose of istiratumab as indicated in Table 2, administered alone or in combination with the PD-1 blocking antibody.

In some embodiments, the method comprises administering an antineoplastic therapy once every two weeks to a human patient diagnosed with cancer, wherein the antineoplastic therapy consists of the administration of 2.8, 2.24 or 1.96 g of istiratumab to the patient, where the human patient does not have a diagnosed auto-immune disease. In another embodiment, the method comprises administering an antineoplastic therapy once every two weeks to a human patient diagnosed with cancer, where the antineoplastic therapy consists of the administration of 2.8, 2.24 or 1.96 g of istiratumab to the patient in combination with 3 mg/kg nivolumab, where the human patient is diagnosed with unresectable or metastatic melanoma and disease progression following ipilumumab and, if BRAF V600 mutation positive, a BRAF inhibitor. Alternatively, the antineoplastic therapy is administered once every week, and consists of the administration of istiratumab at a therapeutically effective dose of 6 mg/kg, 12 mg/kg or 20 mg/kg per dose.

In some embodiments, the antineoplastic therapy is administered once every two weeks to a patient with cancer disease progression after prior treatment with ipilimumab (e.g., melanoma disease progression), where the antineoplastic therapy consists of administration of a therapeutically effective dose of istiratumab (e.g., 40 mg/kg or 2.8 grams fixed dose), alone or in combination with the anti PD-1 antibody (e.g., 3 mg/kg nivolumab as an intravenous infusion over 60 minutes). Istiratumab can be administered as an intravenous infusion, for example over a 90-120 minute time period (e.g., a 90-minute infusion once every two weeks or a 120-minute infusion once every two weeks, or a 120 minute infusion in the first dose, followed by a 90 minute infusion in subsequent doses). In some embodiments, the antineoplastic therapy includes or consists of one or more reductions in the dose of istiratumab as indicated in Table 2, administered alone or in combination with the PD-1 blocking antibody.

In some embodiments, the immunomodulatory therapeutic agent is a programmed cell death-ligand 1 (PD-L1) blocking antibody. The immunomodulatory therapeutic agent can be indicated for the treatment of locally advanced or metastatic urothelial carcinoma, including patients with disease progression during or following platinum-containing chemotherapy and patients having disease progression within 12 months of neoadjuvant or adjuvant treatment with platinum-containing chemotherapy. In some embodiments, cancer is treated by administration of istiratumab in combination with a PD-L1 blocking antibody to a human patient diagnosed with cancer, such as cancer having a solid tumor. In some embodiments, the istiratumab is administered alone or in combination with a PD-L1 blocking antibody (e.g., atezoluzumab) for the treatment of patients with unresectable or metastatic melanoma.

Preferably, the human patient does not have an auto-immune condition raising unacceptable risks of medically undesirable complications from the administration of istiratumab alone or in combination with the PD-L1 blocking antibody. For example, the patients having one or more of the following can be excluded from receiving a combination of istiratumab and the PD-L1 blocking antibody: active autoimmune disease or those receiving systemic immunosuppression for organ transplantation. In some embodiments, patients who have a relevant medical history can also be excluded from receiving the antineoplastic therapy, including patients who have/had: a history of autoimmune disease, active or corticosteroid-dependent brain metastases, administration of a live, attenuated vaccine within 28 days prior to treatment, or administration of systemic immunostimulatory agents or systemic immunosuppressive medications.

In some embodiments, the method of treating cancer comprises administration of an antineoplastic therapy, wherein the antineoplastic therapy comprises or consists of administration of a therapeutically effective dose of istiratumab (e.g., 40 mg/kg or 2.8 grams fixed dose) once every two weeks, alone or in combination with the anti PD-L1 antibody (e.g., 1200 mg atezolizumab once every three weeks). Istiratumab can be administered as an intravenous infusion, for example over a 90-120 minute time period (e.g., a 90-minute infusion once every two weeks or a 120-minute infusion once every two weeks, or a 120 minute infusion in the first dose, followed by a 90 minute infusion in subsequent doses). In some embodiments, the antineoplastic therapy includes or consists of one or more reductions in the dose of istiratumab as indicated in Table 2, administered alone or in combination with the PD-L1 blocking antibody.

Agents that Impede Regulatory T Cell Activity

Agents that impede regulatory T cell activity (Tregs) include small molecule immunomodulatory agents, radiation, anticancer vaccines, and immunomodulatory antibodies that do not bind to human IGF-1. Tregs, formerly known as suppressor T cells, are a subpopulation of T cells which modulate the immune system, suppress immune responses against other cells and maintain tolerance to self-antigens. Generally, these cells suppress or downregulate the induction and proliferation of effector T cells. Therefore, when regulatory T cell activity is impeded, the immune response suppressive activities of these cells will be lessened or removed.

Immunomodulatory Antibodies

In some embodiments, a therapeutically-effective dose of istiratumab (MM-141) is administered in combination with an immunomodulatory antibody. As described above, the immunomodulatory antibody is preferably selected from the group consisting of: a human cytotoxic T-lymphocyte antigen 4 (CTLA-4)-blocking antibody such as ipilimumab, and a human programmed death receptor-1 (PD-1) antibody such as nivolumab. The immunomodulatory agent can be indicated for the safe and effective treatment of unresectable or metastatic melanoma alone or in combination with istiratumab.

Additional examples of immunomodulatory antibodies that do not bind IGF-1 include an immunomodulatory antibody that may be selected from, but is not limited to, the group consisting of (a) an agonistic anti-receptor antibody that immunospecifically binds human OX40, CD40, GITR, CD27, ICOS, or 4-1BB; (b) an antagonistic anti-receptor antibody that immunospecifically binds human CTLA-4 (cyctotoxic T-lymphocyte-associated protein 4, also known as CD152), PD-1 (programmed cell death protein 1, also known as CD279), PD-L1 (programmed death-ligand 1, also known as CD274 or B7-H1), TIM-3, BTLA, VISTA, LAG-3, KIR, CD47, CD25, B7-H3, or B7-H4; and (c) an anti-ligand antibody that blocks the function of IL-6, IL-10, TGFβ, angiopoetin-2, VEGF, IL-17, IL-23, or TNFalpha. In one embodiment, the second agent is an anti-PD-1 antibody, an anti-PD-L1 antibody, or an anti-CTLA-4 antibody. In another embodiment, the second agent is atezolizumab, durvalumab, or avelumab. In another embodiment, the second agent is selected from the group consisting of nivolumab, pembrolizumab, and pidilizumab. In another embodiment, the second agent is ipilimumab or tremelimumab. In another embodiment, the second agent is lirilumab.

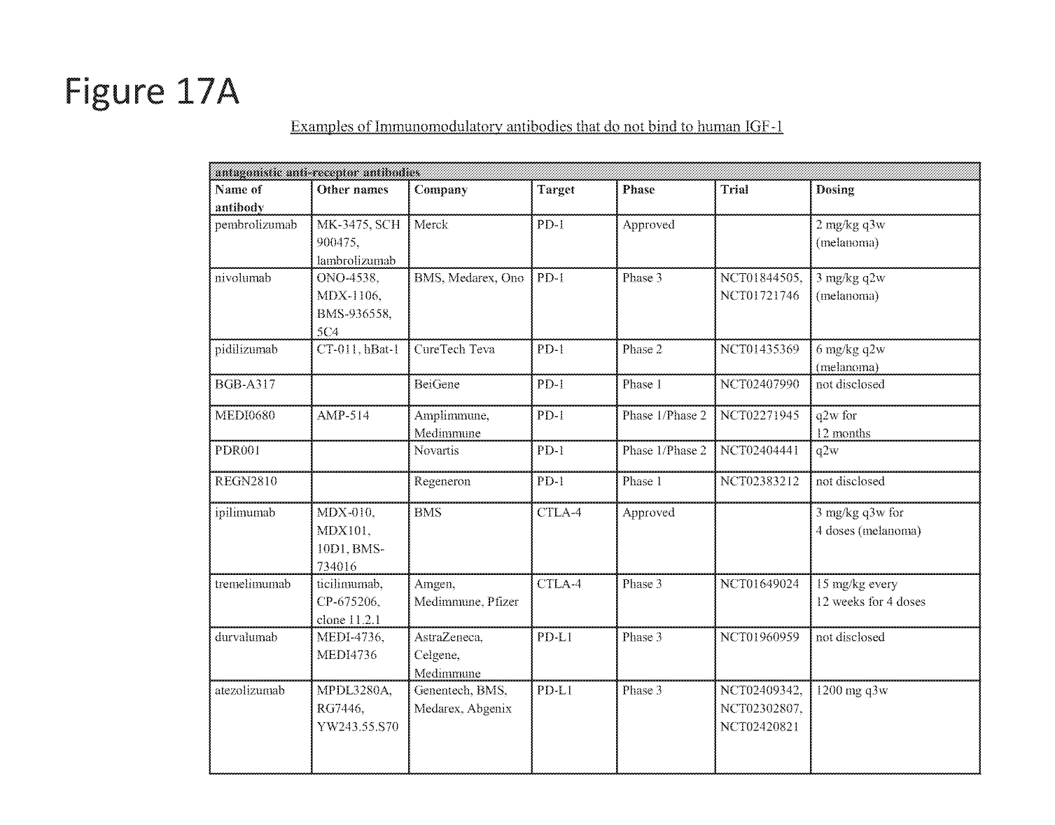

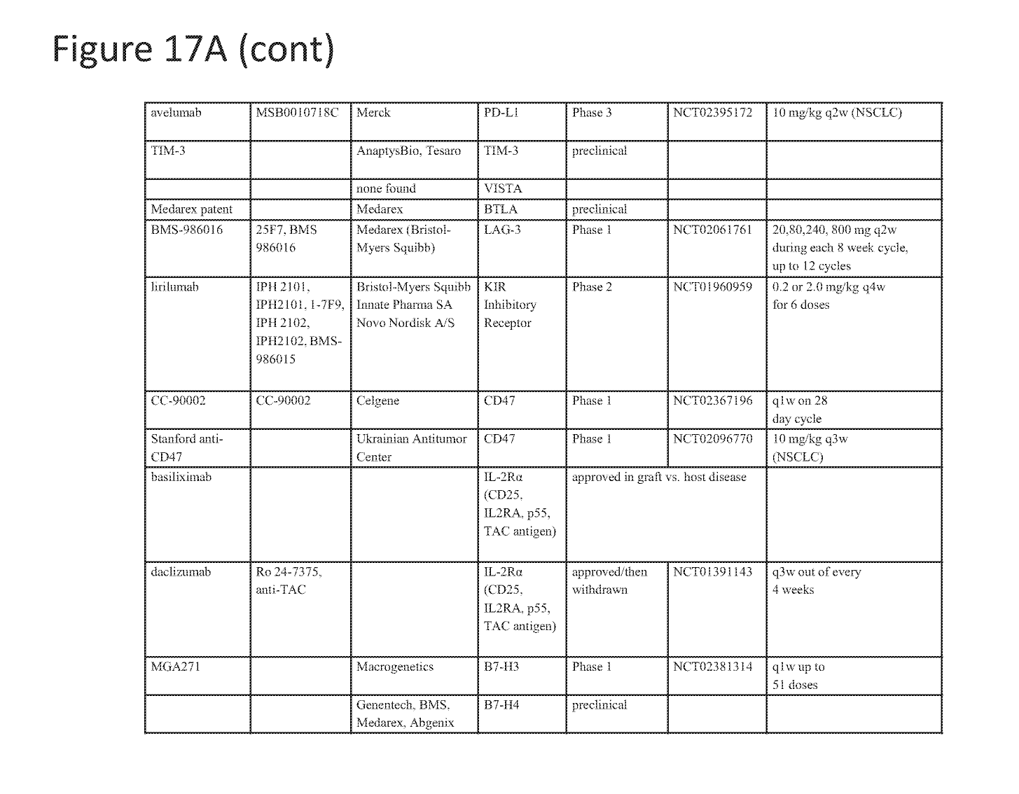

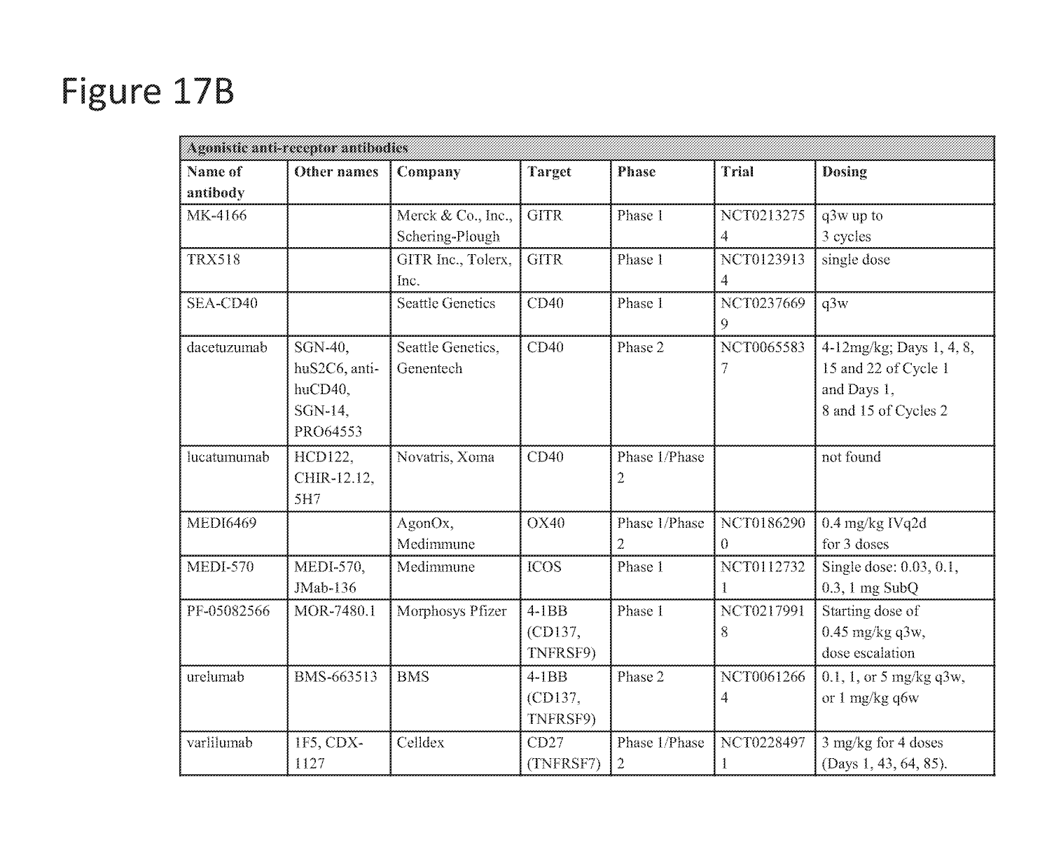

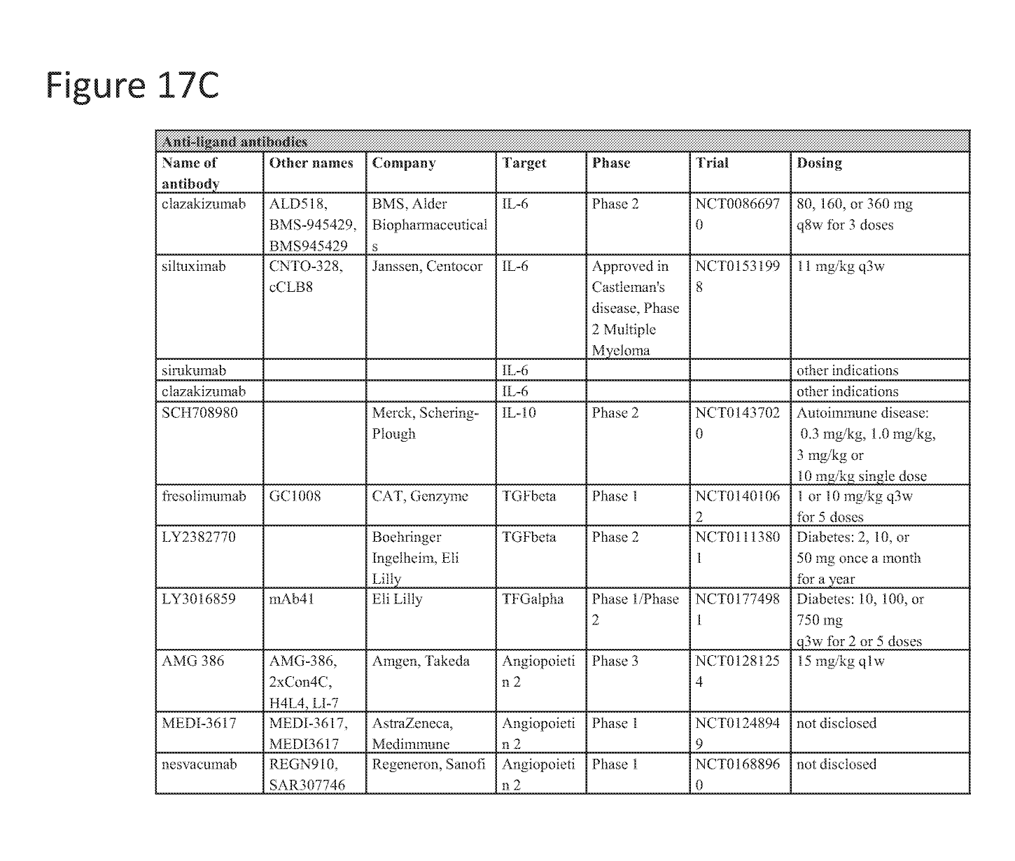

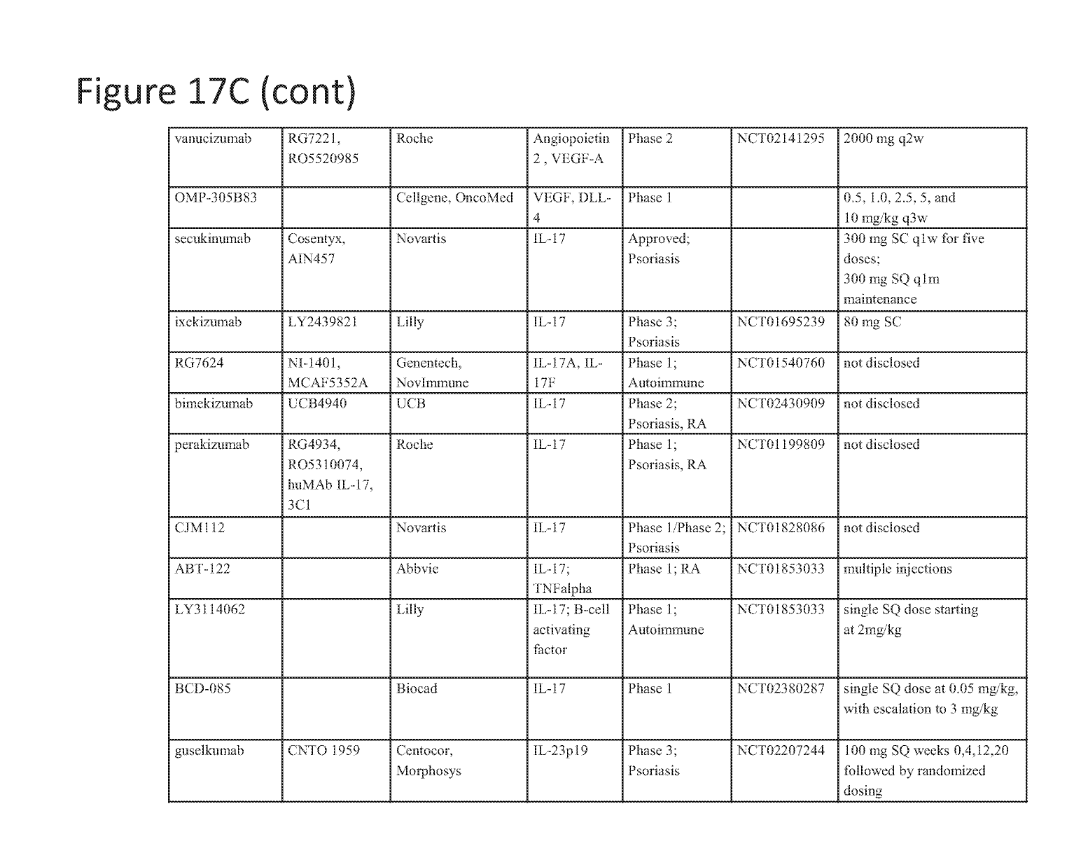

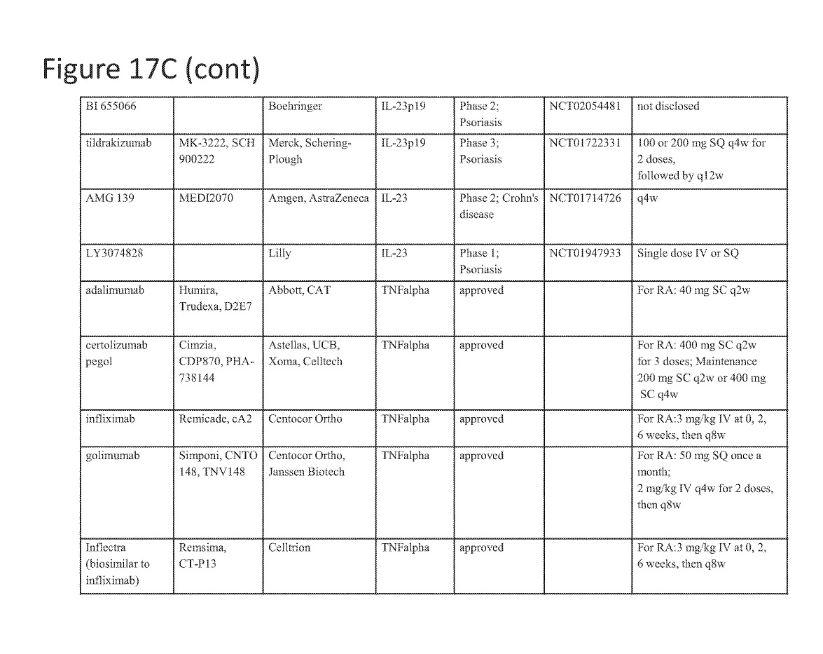

Immunomodulatory antibodies that do not bind to human IGF-1 are further detailed in FIGS. 17A-17C.

Anti-Cancer Vaccines

In one aspect, the therapeutic combinations provided herein that impede regulatory T cell activity are anticancer vaccines. In some embodiments, the anticancer vaccine is selected from, but is not limited to, the group consisting of OncoVex, MAGE-A3, PROSTVAC, GVAX, CDX110, CDX1307, CDX1401, CV9104, BIOVAXID, IMA 901, and ADXS11-001.

Polyvalent Bispecific (Anti-IGF-1R and Anti-ErbB3) Antibodies

In certain embodiments, the bispecific (anti-IGF-1R and anti-ErbB3) antibody for use in the methods disclosed herein is istiratumab (MM-141). In addition to U.S. Pat. No. 8,476,409, referred to above, disclosures regarding bispecific antibodies useful in the methods disclosed herein may be found in U.S. Patent Publication No. 2012-0244163; U.S. Patent Publication No. 2015-0231219 (also see WIPO Publication No. WO 2013/152034); and in WIPO Publication No. WO 2015/130554. Further discussion of bispecific antibodies for use in the disclosed methods are described below. Provided herein are polyvalent bispecific antibodies (“PBAs”), which may be isolated monoclonal antibodies. Exemplary PBAs comprise at least one anti-IGF-1R binding site and at least one anti-ErbB3 binding site or at least two anti-IGF-1R binding sites and at least two anti-ErbB3 binding sites. In a preferred embodiment, the anti-IGF-1R binding site binds specifically to a human IGF-1R and the anti-ErbB3 binding site binds specifically to human ErbB3. In certain embodiments, the PBA comprises two heavy-light chain pairs that associate with each other to form a single protein, wherein each heavy-light chain pair comprises an anti-IGF-1R binding site and an anti-ErbB3 binding site. In certain embodiments, the anti-IGF-1R binding site and the anti-ErbB3 binding site of a first heavy-light chain pair are connected through an immunoglobulin CR that associates with the immunoglobulin CR of another heavy-light chain pair (e.g., by disulfide bonds) to form, e.g., a single IgG-like protein. A preferred PBA as described herein has advantageous properties, such as the ability to inhibit tumor cell proliferation and to reduce or stabilize tumor growth equivalently to or more potently than either its isolated anti-IGF-1R binding moiety or its isolated anti-ErbB3 binding moiety, and in certain embodiments, the ability to inhibit either or both of tumor invasiveness and tumor metastasis. An exemplary PBA described herein can inhibit either or both of IGF-1R and ErbB3 mediated signal transduction, such as IGF-1R, ErbB3 and AKT phosphorylation, equivalently to or more potently than either its isolated anti-IGF-1R binding moiety or its isolated anti-ErbB3 binding moiety. An exemplary PBA will (i) inhibit growth of tumor cells, e.g., by at least 10%, 20%, 30%, 40%, 50%, 60%, 70%, 80%, 90% or more; or (ii) inhibit IGF-1r, ErbB3 or Akt phosphorylation, e.g., by at least 10%, 20%, 30%, 40%, 50%, 60%, 70%, 80%, 90% or more, e.g., to a similar extent or more potently than either its isolated anti-IGF-1R binding moiety or its isolated anti-ErbB3 binding moiety, or both (i) and (ii). An exemplary PBA will (iii) be stable, e.g., be at least 80% monomeric in a solution after 1, 2, 3, 4, 5 or more days at 4° C., room temperature or 37° C., or (iv) have a Tm (e.g., as determined by DSF) of at least 50° C., 55° C., 60° C., 65° C. or more, or both (iii) and (iv).

The methods disclosed herein may comprise the use or administration of one or more polyvalent bispecific antibodies (PBA), which antibodies are proteins comprising two pairs of polypeptide chains, each pair of said two pairs comprising a heavy chain joined to a light chain by at least one heavy-light chain bond; wherein (a) each pair comprises at least one anti-IGF-1R binding site and at least one anti-ErbB3 binding site; and (b) each pair comprises a first binding site that comprises an N-terminal portion of the heavy chain of the PBA and an N-terminal portion of the light chain of the PBA, and a second binding site that is a C-terminal scFv that is entirely comprised by the heavy chain of the PBA, said C-terminal scFv containing a heavy chain VR joined to a light chain VR by an scFv linker; and the anti-IGF-1R binding site is linked to the anti-ErbB3 binding site through a heavy chain immunoglobulin (HC Ig) constant region (CR) comprised by the heavy chain of the PBA, and the two pairs are conjoined by at least one bond between the HC Ig CRs of each pair. In preferred embodiments, the anti-IGF-1R binding site comprises a heavy chain variable (VH) domain comprising a set of three VH Complementarity Determining Regions (CDRs) comprising either (a) VHCDR1 (aa numbers 26-35), VHCDR2 (aa numbers 51-66), and VHCDR3 (aa numbers 99-111), of a heavy chain having an aa sequence comprising the aa sequence of (set forth in) a SEQ ID NO selected from the group consisting of SEQ ID NO: 1, SEQ ID NOs:8-31 and SEQ ID NOs:384-385; or (b) a set of three VH Complementarity Determining Regions (CDRs) comprising VHCDR1 comprising SEQ ID NO:302, VHCDR2 comprising SEQ ID NO:303 and VHCDR3 comprising SEQ ID NO:304, and a light chain variable (VL) domain comprising a set of three VLCDRs comprising either (c) VLCDR1 (aa numbers 24-34), VLCDR2 (aa numbers 50-56) and VLCDR3 (aa numbers 89-97) of a light chain having an aa sequence comprising the aa sequence of a SEQ ID NO selected from the group consisting of SEQ ID NOs:2-3, SEQ ID NOs:32-133, and SEQ ID NOs:386-387; or (d) a set of three VLCDRs comprising VLCDR1 comprising SEQ ID NO:305, VLCDR2 comprising SEQ ID NO:306 and VLCDR3 comprising SEQ ID NO:307 or SEQ ID NO:308, and each CDR further comprising an amino terminus and a carboxy terminus, wherein the CDRs of each set of CDRs are arranged in the corresponding heavy or light chain in a linear amino to carboxy order of CDR1, CDR2 and CDR3, or wherein the sequences of VHCDR1, VHCDR2 and VHCDR3 comprise variable aas, and the sequences of VLCDR1, VLCDR2 and VLCDR3 comprise variable aas, with the proviso that the PBA (i) does not comprise both the anti-IGF-1R SF module and the anti-ErbB3 C8 module; or (ii) comprises at least one CDR or FR that differs in one or more aas from a CDR or FR, respectively, of the SF or C8 module. In certain embodiments, the anti-ErbB3 binding site comprises a VH domain comprising a set of three VH CDRs comprising either (e) VHCDR1 (aa numbers 26-35), VHCDR2 (aa numbers 51-66) and VHCDR3 (aa numbers 99-111) of a heavy chain having an aa sequence comprising the aa sequence of a SEQ ID NO selected from the group consisting of SEQ ID NOs:4-5, SEQ ID NOs: 134-165, and SEQ ID NO:388, or (f) a set of three VH CDRs comprising VHCDR1 comprising SEQ ID NO:309, VHCDR2 comprising SEQ ID NO:310 and VHCDR3 comprising SEQ ID NO:311, and a light chain variable (VL) domain comprising a set of three VLCDRs comprising either (g) VLCDR1 (aa numbers 23-33), VLCDR2 (aa numbers 49-55) and VLCDR3 (aa numbers 88-98), of a light chain having an aa sequence comprising the aa sequence of a SEQ ID NO selected from the group consisting of SEQ ID NOs:6-7 and SEQ ID NOs: 166-200; or (h) a light chain variable (VL) domain comprising a set of three VLCDRs comprising VLCDR1 comprising SEQ ID NO:312, VLCDR2 comprising SEQ ID NO:313 and VLCDR3 comprising SEQ ID NO:314 or SEQ ID NO:315, and each CDR further comprises an amino terminus and a carboxy terminus, wherein the CDRs of each set of CDRs are arranged in the antibody in a linear amino to carboxy order of CDR1, CDR2 and CDR3, or wherein the sequences of the VHCDR1, VHCDR2 and VHCDR3 comprise variable aas, and the sequences of the VLCDR1, VLCDR2 and VLCDR3 comprise variable aas, wherein the PBA (i) does not comprise both the anti-IGF-1R module comprising a light chain comprising SEQ ID NO:35 and a heavy chain comprising SEQ ID NO:11 and the b) an anti-ErbB3 C8 module comprising a light chain comprising SEQ ID NO: 175 and a heavy chain comprising SEQ ID NO: 145. In certain embodiments, the anti-IGF-1R VLCDR3 comprises SEQ ID NO:308 or the anti-ErbB3 VLCDR3 comprises SEQ ID NO:315. In certain embodiments, the two pairs of polypeptide chains are have essentially identical sequences. At least one of at least one bonds between the HC Ig CRs bonds is a disulfide bond and may be a disulfide bond or a van der Waals bond or at least one of said at least one heavy-light chain bonds is a disulfide bond and may be a disulfide bond or a van der Waals bond. In certain embodiments, the anti-ErbB3 binding site is the C-terminal scFv and in certain embodiments, the anti-IGF-1R binding site is the C-terminal scFv. The anti-IGF-1R binding site, the HC Ig CR and the anti-ErbB3 binding site of a PBA may comprise the heavy chain of that pair, which is comprised by a single, contiguous polypeptide chain.

The one or more PBA may (i) inhibit growth of tumor cells in vitro at a concentration of 1 μM or less, or 100 nM or less, or 10 nM or less, or 1 nM or less, or ii) inhibits either or both of heregulin and IGF1 induced signal transduction with an IC50 of 10 nM or less or 1 nM or less or 100 pM or less, or a maximal percent inhibition of at least 70% or at least 80% or at least 90%, as indicated by inhibition of phosphorylation of either or both of pErbB3 and pIGF-1R. Growth inhibition may be measured with a CTG assay in DU145 cells in culture. Inhibition of signal transduction may be determined in BxPC-3 cells in culture following stimulation with IGF-1 at 80 ng/ml and heregulin at 20 ng/ml for 15 minutes.

In certain embodiments, each HC Ig CR of a PBA comprises a CH3 domain that mediates conjunction with the CH3 domain of the other pair. Each HC Ig CR may also comprise a CH2 domain, a hinge, and a CH1 domain. In certain embodiments, the CH1 domain of a PBA is linked at its C-terminus to the N-terminus of a hinge, which is linked at its C-terminus to the N-terminus of a CH2 domain, which is linked at its C-terminus to the N-terminus of a CH3 domain.

Each first binding site may comprise a first VH domain, and each CH1 domain of a PBA may be linked at its N-terminus to the C-terminus of the first VH domain. Each CH3 domain of a PBA may be linked at its C-terminus to the N-terminus of the scFv. Each CH3 domain of a PBA may be linked at its C terminus to the N-terminus of a connecting linker, which is linked at its C-terminus to the N-terminus of the scFv. Each light chain may comprise a first VL domain that associates with the first VH domain to form the first binding site. Each first VL domain may be linked at its C-terminus to the N-terminus of a CL domain. Each first binding site may be an anti-IGF-1R binding site and each scFv may be an anti-ErbB3 scFv. Each first binding site may be an anti-ErbB3 binding site and each scFv may be an anti-IGF-1R scFv. The HC Ig CR of a PBA may be an IgG CR, e.g., an IgG1 or IgG2 CR.

In certain embodiments, the anti-IGF-1R VHCDR1, VHCDR2, VHCDR3 of a PBA comprise the aa sequence of the corresponding CDRs of SEQ ID NO:8 and the anti-IGF-1R VLCDR1, VLCDR2 and VLCDR3 of the PBA comprise the aa sequence of the corresponding CDRs of SEQ ID NO:32. In certain embodiments, the anti-IGF-1R VHCDR1, VHCDR2, VHCDR3 of a PBA comprise the aa sequence of the corresponding CDRs of SEQ ID NO:9 and the anti-IGF-1R VLCDR1, VLCDR2 and VLCDR3 of the PBA comprise the aa sequence of the corresponding CDRs of SEQ ID NO:33. In certain embodiments, the anti-IGF-1R VHCDR1, VHCDR2, VHCDR3 of a PBA comprise the aa sequence of the corresponding CDRs of SEQ ID NO:10 and the anti-IGF-1R VLCDR1, VLCDR2 and VLCDR3 of the PBA comprise the aa sequence of the corresponding CDRs of SEQ ID NO:34. In certain embodiments, the anti-IGF-1R VHCDR1, VHCDR2, VHCDR3 of a PBA comprise the aa sequence of the corresponding CDRs of SEQ ID NO:11 and the anti-IGF-1R VLCDR1, VLCDR2 and VLCDR3 of the PBA comprise the aa sequence of the corresponding CDRs of SEQ ID NO:35. In certain embodiments, the anti-IGF-1R VHCDR1, VHCDR2, VHCDR3 of a PBA comprise the aa sequence of the corresponding CDRs of SEQ ID NO:8 and the anti-IGF-1R VLCDR1, VLCDR2 and VLCDR3 of the PBA comprise the aa sequence of the corresponding CDRs of SEQ ID NO:33. In certain embodiments, the anti-IGF-1R VHCDR1, VHCDR2, VHCDR3 of a PBA comprise the aa sequence of the corresponding CDRs of SEQ ID NO:10 and the anti-IGF-1R VLCDR1, VLCDR2 and VLCDR3 of the PBA comprise the aa sequence of the corresponding CDRs of SEQ ID NO:32.

In certain embodiments, the anti-ErbB3 VHCDR1, VHCDR2, VHCDR3 of a PBA comprise the aa sequence of the corresponding CDRs of SEQ ID NO:134 and the anti-ErbB3 VLCDR1, VLCDR2 and VLCDR3 of the PBA comprise the aa sequence of the corresponding CDRs of SEQ ID NO: 166. In certain embodiments, the anti-ErbB3 VHCDR1, VHCDR2, VHCDR3 of a PBA comprise the aa sequence of the corresponding CDRs of SEQ ID NO:135 and the anti-ErbB3 VLCDR1, VLCDR2 and VLCDR3 of the PBA comprise the aa sequence of the corresponding CDRs of SEQ ID NO:167. In certain embodiments, the anti-ErbB3 VHCDR1, VHCDR2, VHCDR3 of a PBA comprise the aa sequence of the corresponding CDRs of SEQ ID NO:136 and the anti-ErbB3 VLCDR1, VLCDR2 and VLCDR3 of the PBA comprise the aa sequence of the corresponding CDRs of SEQ ID NO: 168. In certain embodiments, the anti-ErbB3 VHCDR1, VHCDR2, VHCDR3 of a PBA comprise the aa sequence of the corresponding CDRs of SEQ ID NO:137 and the anti-ErbB3 VLCDR1, VLCDR2 and VLCDR3 of the PBA comprise the aa sequence of the corresponding CDRs of SEQ ID NO: 169. In certain embodiments, the anti-ErbB3 VHCDR1, VHCDR2, VHCDR3 of a PBA comprise the aa sequence of the corresponding CDRs of SEQ ID NO:138 and the anti-ErbB3 VLCDR1, VLCDR2 and VLCDR3 of the PBA comprise the aa sequence of the corresponding CDRs of SEQ ID NO: 170. In certain embodiments, the anti-ErbB3 VHCDR1, VHCDR2, VHCDR3 of a PBA comprise the aa sequence of the corresponding CDRs of SEQ ID NO:139 and the anti-ErbB3 VLCDR1, VLCDR2 and VLCDR3 of the PBA comprise the aa sequence of the corresponding CDRs of SEQ ID NO:171. In certain embodiments, the anti-ErbB3 VHCDR1, VHCDR2, VHCDR3 of a PBA comprise the aa sequence of the corresponding CDRs of SEQ ID NO: 140 and the anti-ErbB3 VLCDR1, VLCDR2 and VLCDR3 of the PBA comprise the aa sequence of the corresponding CDRs of SEQ ID NO:172. In certain embodiments, the anti-ErbB3 VHCDR1, VHCDR2, VHCDR3 of a PBA comprise the aa sequence of the corresponding CDRs of SEQ ID NO: 141 and the anti-ErbB3 VLCDR1, VLCDR2 and VLCDR3 of the PBA comprise the aa sequence of the corresponding CDRs of SEQ ID NOs: 173. In certain embodiments, the anti-ErbB3 VHCDR1, VHCDR2, VHCDR3 of a PBA comprise the aa sequence of the corresponding CDRs of SEQ ID NO: 142 and the anti-ErbB3 VLCDR1, VLCDR2 and VLCDR3 of the PBA comprise the aa sequence of the corresponding CDRs of SEQ ID NO:174. In certain embodiments, the anti-ErbB3 VHCDR1, VHCDR2, VHCDR3 of a PBA comprise the aa sequence of the corresponding CDRs of SEQ ID NO:143 and the anti-ErbB3 VLCDR1, VLCDR2 and VLCDR3 of the PBA comprise the aa sequence of the corresponding CDRs of SEQ ID NO: 175. In certain embodiments, the anti-ErbB3 VHCDR1, VHCDR2, VHCDR3 of a PBA comprise the aa sequence of the corresponding CDRs of SEQ ID NO:136 and the anti-ErbB3 VLCDR1, VLCDR2 and VLCDR3 of the PBA comprise the aa sequence of the corresponding CDRs of SEQ ID NO:169.

In certain embodiments, the anti-IGF-1R VHCDR1, VHCDR2, VHCDR3 of a PBA comprise the aa sequence of the corresponding CDRs of SEQ ID NO:8 and the anti-IGF-1R VLCDR1, VLCDR2 and VLCDR3 of the PBA comprise the aa sequence of the corresponding CDRs of SEQ ID NO:32; and (a) the anti-ErbB3 VHCDR1, VHCDR2, VHCDR3 of the PBA comprise the aa sequence of the corresponding CDRs of SEQ ID NO:134 and the anti-ErbB3 VLCDR1, VLCDR2 and VLCDR3 of the PBA comprise the aa sequence of the corresponding CDRs of SEQ ID NO: 166; or (b) the anti-ErbB3 VHCDR1, VHCDR2, VHCDR3 of the PBA comprise the aa sequence of the corresponding CDRs of SEQ ID NO:135 and the anti-ErbB3 VLCDR1, VLCDR2 and VLCDR3 of the PBA comprise the aa sequence of the corresponding CDRs of SEQ ID NO:167; or (c) the anti-ErbB3 VHCDR1, VHCDR2, VHCDR3 of the PBA comprise the aa sequence of the corresponding CDRs of SEQ ID NO: 136 and the anti-ErbB3 VLCDR1, VLCDR2 and VLCDR3 of the PBA comprise the aa sequence of the corresponding CDRs of SEQ ID NO: 168; or (d) the anti-ErbB3 VHCDR1, VHCDR2, VHCDR3 of the PBA comprise the aa sequence of the corresponding CDRs of SEQ ID NO:137 and the anti-ErbB3 VLCDR1, VLCDR2 and VLCDR3 of the PBA comprise the aa sequence of the corresponding CDRs of SEQ ID NO: 169; or (e) the anti-ErbB3 VHCDR1, VHCDR2, VHCDR3 of the PBA comprise the aa sequence of the corresponding CDRs of SEQ ID NO:138 and the anti-ErbB3 VLCDR1, VLCDR2 and VLCDR3 of the PBA comprise the aa sequence of the corresponding CDRs of SEQ ID NO: 170; or (f) the anti-ErbB3 VHCDR1, VHCDR2, VHCDR3 of the PBA comprise the aa sequence of the corresponding CDRs of SEQ ID NO:139 and the anti-ErbB3 VLCDR1, VLCDR2 and VLCDR3 of the PBA comprise the aa sequence of the corresponding CDRs of SEQ ID NO:171; or (g) the anti-ErbB3 VHCDR1, VHCDR2, VHCDR3 of the PBA comprise the aa sequence of the corresponding CDRs of SEQ ID NO: 140 and the anti-ErbB3 VLCDR1, VLCDR2 and VLCDR3 of the PBA comprise the aa sequence of the corresponding CDRs of SEQ ID NO: 172; or (h) the anti-ErbB3 VHCDR1, VHCDR2, VHCDR3 of the PBA comprise the aa sequence of the corresponding CDRs of SEQ ID NO: 141 and the anti-ErbB3 VLCDR1, VLCDR2 and VLCDR3 of the PBA comprise the aa sequence of the corresponding CDRs of SEQ ID NOs: 173; or (i) the anti-ErbB3 VHCDR1, VHCDR2, VHCDR3 of the PBA comprise the aa sequence of the corresponding CDRs of SEQ ID NO: 142 and the anti-ErbB3 VLCDR1, VLCDR2 and VLCDR3 of the PBA comprise the aa sequence of the corresponding CDRs of SEQ ID NO: 174; or (j) the anti-ErbB3 VHCDR1, VHCDR2, VHCDR3 of the PBA comprise the aa sequence of the corresponding CDRs of SEQ ID NO:143 and the anti-ErbB3 VLCDR1, VLCDR2 and VLCDR3 of the PBA comprise the aa sequence of the corresponding CDRs of SEQ ID NO:175; or (k) the anti-ErbB3 VHCDR1, VHCDR2, VHCDR3 of the PBA comprise the aa sequence of the corresponding CDRs of SEQ ID NO: 136 and the anti-ErbB3 VLCDR1, VLCDR2 and VLCDR3 of the PBA comprise the aa sequence of the corresponding CDRs of SEQ ID NO:169.

In certain embodiments, the anti-IGF-1R VHCDR1, VHCDR2, VHCDR3 of a PBA comprise the aa sequence of the corresponding CDRs of SEQ ID NO:9 and the anti-IGF-1R VLCDR1, VLCDR2 and VLCDR3 of the PBA comprise the aa sequence of the corresponding CDRs of SEQ ID NO:33; and (a) the anti-ErbB3 VHCDR1, VHCDR2, VHCDR3 of the PBA comprise the aa sequence of the corresponding CDRs of SEQ ID NO:134 and the anti-ErbB3 VLCDR1, VLCDR2 and VLCDR3 of the PBA comprise the aa sequence of the corresponding CDRs of SEQ ID NO: 166; or (b) the anti-ErbB3 VHCDR1, VHCDR2, VHCDR3 of the PBA comprise the aa sequence of the corresponding CDRs of SEQ ID NO:135 and the anti-ErbB3 VLCDR1, VLCDR2 and VLCDR3 of the PBA comprise the aa sequence of the corresponding CDRs of SEQ ID NO:167; or (c) the anti-ErbB3 VHCDR1, VHCDR2, VHCDR3 of the PBA comprise the aa sequence of the corresponding CDRs of SEQ ID NO: 136 and the anti-ErbB3 VLCDR1, VLCDR2 and VLCDR3 of the PBA comprise the aa sequence of the corresponding CDRs of SEQ ID NO:168; or (d) the anti-ErbB3 VHCDR1, VHCDR2, VHCDR3 of the PBA comprise the aa sequence of the corresponding CDRs of SEQ ID NO:137 and the anti-ErbB3 VLCDR1, VLCDR2 and VLCDR3 of the PBA comprise the aa sequence of the corresponding CDRs of SEQ ID NO: 169; or (e) the anti-ErbB3 VHCDR1, VHCDR2, VHCDR3 of the PBA comprise the aa sequence of the corresponding CDRs of SEQ ID NO:138 and the anti-ErbB3 VLCDR1, VLCDR2 and VLCDR3 of the PBA comprise the aa sequence of the corresponding CDRs of SEQ ID NO: 170; or (f) the anti-ErbB3 VHCDR1, VHCDR2, VHCDR3 of the PBA comprise the aa sequence of the corresponding CDRs of SEQ ID NO:139 and the anti-ErbB3 VLCDR1, VLCDR2 and VLCDR3 of the PBA comprise the aa sequence of the corresponding CDRs of SEQ ID NO:171; or (g) the anti-ErbB3 VHCDR1, VHCDR2, VHCDR3 of the PBA comprise the aa sequence of the corresponding CDRs of SEQ ID NO: 140 and the anti-ErbB3 VLCDR1, VLCDR2 and VLCDR3 of the PBA comprise the aa sequence of the corresponding CDRs of SEQ ID NO: 172; or (h) the anti-ErbB3 VHCDR1, VHCDR2, VHCDR3 of the PBA comprise the aa sequence of the corresponding CDRs of SEQ ID NO: 141 and the anti-ErbB3 VLCDR1, VLCDR2 and VLCDR3 of the PBA comprise the aa sequence of the corresponding CDRs of SEQ ID NOs: 173; or (i) the anti-ErbB3 VHCDR1, VHCDR2, VHCDR3 of the PBA comprise the aa sequence of the corresponding CDRs of SEQ ID NO: 142 and the anti-ErbB3 VLCDR1, VLCDR2 and VLCDR3 of the PBA comprise the aa sequence of the corresponding CDRs of SEQ ID NO: 174; or (j) the anti-ErbB3 VHCDR1, VHCDR2, VHCDR3 of the PBA comprise the aa sequence of the corresponding CDRs of SEQ ID NO:143 and the anti-ErbB3 VLCDR1, VLCDR2 and VLCDR3 of the PBA comprise the aa sequence of the corresponding CDRs of SEQ ID NO:175; or (k) the anti-ErbB3 VHCDR1, VHCDR2, VHCDR3 of the PBA comprise the aa sequence of the corresponding CDRs of SEQ ID NO: 136 and the anti-ErbB3 VLCDR1, VLCDR2 and VLCDR3 of the PBA comprise the aa sequence of the corresponding CDRs of SEQ ID NO:169.

In certain embodiments, the anti-IGF-1R VHCDR1, VHCDR2, VHCDR3 of a PBA comprise the aa sequence of the corresponding CDRs of SEQ ID NO:10 and the anti-IGF-1R VLCDR1, VLCDR2 and VLCDR3 of the PBA comprise the aa sequence of the corresponding CDRs of SEQ ID NO:34; and (a) the anti-ErbB3 VHCDR1, VHCDR2, VHCDR3 of the PBA comprise the aa sequence of the corresponding CDRs of SEQ ID NO:134 and the anti-ErbB3 VLCDR1, VLCDR2 and VLCDR3 of the PBA comprise the aa sequence of the corresponding CDRs of SEQ ID NO: 166; or (b) the anti-ErbB3 VHCDR1, VHCDR2, VHCDR3 of the PBA comprise the aa sequence of the corresponding CDRs of SEQ ID NO:135 and the anti-ErbB3 VLCDR1, VLCDR2 and VLCDR3 of the PBA comprise the aa sequence of the corresponding CDRs of SEQ ID NO:167; or (c) the anti-ErbB3 VHCDR1, VHCDR2, VHCDR3 of the PBA comprise the aa sequence of the corresponding CDRs of SEQ ID NO: 136 and the anti-ErbB3 VLCDR1, VLCDR2 and VLCDR3 of the PBA comprise the aa sequence of the corresponding CDRs of SEQ ID NO: 168; or (d) the anti-ErbB3 VHCDR1, VHCDR2, VHCDR3 of the PBA comprise the aa sequence of the corresponding CDRs of SEQ ID NO:137 and the anti-ErbB3 VLCDR1, VLCDR2 and VLCDR3 of the PBA comprise the aa sequence of the corresponding CDRs of SEQ ID NO: 169; or (e) the anti-ErbB3 VHCDR1, VHCDR2, VHCDR3 of the PBA comprise the aa sequence of the corresponding CDRs of SEQ ID NO:138 and the anti-ErbB3 VLCDR1, VLCDR2 and VLCDR3 of the PBA comprise the aa sequence of the corresponding CDRs of SEQ ID NO: 170; or (f) the anti-ErbB3 VHCDR1, VHCDR2, VHCDR3 of the PBA comprise the aa sequence of the corresponding CDRs of SEQ ID NO:139 and the anti-ErbB3 VLCDR1, VLCDR2 and VLCDR3 of the PBA comprise the aa sequence of the corresponding CDRs of SEQ ID NO:171; or (g) the anti-ErbB3 VHCDR1, VHCDR2, VHCDR3 of the PBA comprise the aa sequence of the corresponding CDRs of SEQ ID NO: 140 and the anti-ErbB3 VLCDR1, VLCDR2 and VLCDR3 of the PBA comprise the aa sequence of the corresponding CDRs of SEQ ID NO: 172; or (h) the anti-ErbB3 VHCDR1, VHCDR2, VHCDR3 of the PBA comprise the aa sequence of the corresponding CDRs of SEQ ID NO: 141 and the anti-ErbB3 VLCDR1, VLCDR2 and VLCDR3 of the PBA comprise the aa sequence of the corresponding CDRs of SEQ ID NOs: 173; or (i) the anti-ErbB3 VHCDR1, VHCDR2, VHCDR3 of the PBA comprise the aa sequence of the corresponding CDRs of SEQ ID NO: 142 and the anti-ErbB3 VLCDR1, VLCDR2 and VLCDR3 of the PBA comprise the aa sequence of the corresponding CDRs of SEQ ID NO: 174; or (j) the anti-ErbB3 VHCDR1, VHCDR2, VHCDR3 of the PBA comprise the aa sequence of the corresponding CDRs of SEQ ID NO:143 and the anti-ErbB3 VLCDR1, VLCDR2 and VLCDR3 of the PBA comprise the aa sequence of the corresponding CDRs of SEQ ID NO:175; or (k) the anti-ErbB3 VHCDR1, VHCDR2, VHCDR3 of the PBA comprise the aa sequence of the corresponding CDRs of SEQ ID NO: 136 and the anti-ErbB3 VLCDR1, VLCDR2 and VLCDR3 of the PBA comprise the aa sequence of the corresponding CDRs of SEQ ID NO:169.

In certain embodiments, the anti-IGF-1R VHCDR1, VHCDR2, VHCDR3 of a PBA comprise the aa sequence of the corresponding CDRs of SEQ ID NO:11 and the anti-IGF-1R VLCDR1, VLCDR2 and VLCDR3 of the PBA comprise the aa sequence of the corresponding CDRs of SEQ ID NO:35; and (a) the anti-ErbB3 VHCDR1, VHCDR2, VHCDR3 of the PBA comprise the aa sequence of the corresponding CDRs of SEQ ID NO:134 and the anti-ErbB3 VLCDR1, VLCDR2 and VLCDR3 of the PBA comprise the aa sequence of the corresponding CDRs of SEQ ID NO: 166; or (b) the anti-ErbB3 VHCDR1, VHCDR2, VHCDR3 of the PBA comprise the aa sequence of the corresponding CDRs of SEQ ID NO:135 and the anti-ErbB3 VLCDR1, VLCDR2 and VLCDR3 of the PBA comprise the aa sequence of the corresponding CDRs of SEQ ID NO:167; or (c) the anti-ErbB3 VHCDR1, VHCDR2, VHCDR3 of the PBA comprise the aa sequence of the corresponding CDRs of SEQ ID NO: 136 and the anti-ErbB3 VLCDR1, VLCDR2 and VLCDR3 of the PBA comprise the aa sequence of the corresponding CDRs of SEQ ID NO: 168; or (d) the anti-ErbB3 VHCDR1, VHCDR2, VHCDR3 of the PBA comprise the aa sequence of the corresponding CDRs of SEQ ID NO:137 and the anti-ErbB3 VLCDR1, VLCDR2 and VLCDR3 of the PBA comprise the aa sequence of the corresponding CDRs of SEQ ID NO: 169; or (e) the anti-ErbB3 VHCDR1, VHCDR2, VHCDR3 of the PBA comprise the aa sequence of the corresponding CDRs of SEQ ID NO:138 and the anti-ErbB3 VLCDR1, VLCDR2 and VLCDR3 of the PBA comprise the aa sequence of the corresponding CDRs of SEQ ID NO: 170; or (f) the anti-ErbB3 VHCDR1, VHCDR2, VHCDR3 of the PBA comprise the aa sequence of the corresponding CDRs of SEQ ID NO:139 and the anti-ErbB3 VLCDR1, VLCDR2 and VLCDR3 of the PBA comprise the aa sequence of the corresponding CDRs of SEQ ID NO:171; or (g) the anti-ErbB3 VHCDR1, VHCDR2, VHCDR3 of the PBA comprise the aa sequence of the corresponding CDRs of SEQ ID NO: 140 and the anti-ErbB3 VLCDR1, VLCDR2 and VLCDR3 of the PBA comprise the aa sequence of the corresponding CDRs of SEQ ID NO: 172; or (h) the anti-ErbB3 VHCDR1, VHCDR2, VHCDR3 of the PBA comprise the aa sequence of the corresponding CDRs of SEQ ID NO: 141 and the anti-ErbB3 VLCDR1, VLCDR2 and VLCDR3 of the PBA comprise the aa sequence of the corresponding CDRs of SEQ ID NOs: 173; or (i) the anti-ErbB3 VHCDR1, VHCDR2, VHCDR3 of the PBA comprise the aa sequence of the corresponding CDRs of SEQ ID NO: 142 and the anti-ErbB3 VLCDR1, VLCDR2 and VLCDR3 of the PBA comprise the aa sequence of the corresponding CDRs of SEQ ID NO: 174; or (j) the anti-ErbB3 VHCDR1, VHCDR2, VHCDR3 of the PBA comprise the aa sequence of the corresponding CDRs of SEQ ID NO:136 and the anti-ErbB3 VLCDR1, VLCDR2 and VLCDR3 of the PBA comprise the aa sequence of the corresponding CDRs of SEQ ID NO: 169.

In certain embodiments, the anti-IGF-1R VHCDR1, VHCDR2, VHCDR3 of a PBA comprise the aa sequence of the corresponding CDRs of SEQ ID NO:8 and the anti-IGF-1R VLCDR1, VLCDR2 and VLCDR3 of the PBA comprise the aa sequence of the corresponding CDRs of SEQ ID NO:33; and (a) the anti-ErbB3 VHCDR1, VHCDR2, VHCDR3 of the PBA comprise the aa sequence of the corresponding CDRs of SEQ ID NO:134 and the anti-ErbB3 VLCDR1, VLCDR2 and VLCDR3 of the PBA comprise the aa sequence of the corresponding CDRs of SEQ ID NO: 166; or (b) the anti-ErbB3 VHCDR1, VHCDR2, VHCDR3 of the PBA comprise the aa sequence of the corresponding CDRs of SEQ ID NO:135 and the anti-ErbB3 VLCDR1, VLCDR2 and VLCDR3 of the PBA comprise the aa sequence of the corresponding CDRs of SEQ ID NO:167; or (c) the anti-ErbB3 VHCDR1, VHCDR2, VHCDR3 of the PBA comprise the aa sequence of the corresponding CDRs of SEQ ID NO: 136 and the anti-ErbB3 VLCDR1, VLCDR2 and VLCDR3 of the PBA comprise the aa sequence of the corresponding CDRs of SEQ ID NO:168; or (d) the anti-ErbB3 VHCDR1, VHCDR2, VHCDR3 of the PBA comprise the aa sequence of the corresponding CDRs of SEQ ID NO:137 and the anti-ErbB3 VLCDR1, VLCDR2 and VLCDR3 of the PBA comprise the aa sequence of the corresponding CDRs of SEQ ID NO: 169; or (e) the anti-ErbB3 VHCDR1, VHCDR2, VHCDR3 of the PBA comprise the aa sequence of the corresponding CDRs of SEQ ID NO:138 and the anti-ErbB3 VLCDR1, VLCDR2 and VLCDR3 of the PBA comprise the aa sequence of the corresponding CDRs of SEQ ID NO: 170; or (f) the anti-ErbB3 VHCDR1, VHCDR2, VHCDR3 of the PBA comprise the aa sequence of the corresponding CDRs of SEQ ID NO:139 and the anti-ErbB3 VLCDR1, VLCDR2 and VLCDR3 of the PBA comprise the aa sequence of the corresponding CDRs of SEQ ID NO:171; or (g) the anti-ErbB3 VHCDR1, VHCDR2, VHCDR3 of the PBA comprise the aa sequence of the corresponding CDRs of SEQ ID NO: 140 and the anti-ErbB3 VLCDR1, VLCDR2 and VLCDR3 of the PBA comprise the aa sequence of the corresponding CDRs of SEQ ID NO: 172; or (h) the anti-ErbB3 VHCDR1, VHCDR2, VHCDR3 of the PBA comprise the aa sequence of the corresponding CDRs of SEQ ID NO: 141 and the anti-ErbB3 VLCDR1, VLCDR2 and VLCDR3 of the PBA comprise the aa sequence of the corresponding CDRs of SEQ ID NOs: 173; or (i) the anti-ErbB3 VHCDR1, VHCDR2, VHCDR3 of the PBA comprise the aa sequence of the corresponding CDRs of SEQ ID NO: 142 and the anti-ErbB3 VLCDR1, VLCDR2 and VLCDR3 of the PBA comprise the aa sequence of the corresponding CDRs of SEQ ID NO: 174; or (j) the anti-ErbB3 VHCDR1, VHCDR2, VHCDR3 of the PBA comprise the aa sequence of the corresponding CDRs of SEQ ID NO:143 and the anti-ErbB3 VLCDR1, VLCDR2 and VLCDR3 of the PBA comprise the aa sequence of the corresponding CDRs of SEQ ID NO:175; or (k) the anti-ErbB3 VHCDR1, VHCDR2, VHCDR3 of the PBA comprise the aa sequence of the corresponding CDRs of SEQ ID NO: 136 and the anti-ErbB3 VLCDR1, VLCDR2 and VLCDR3 of the PBA comprise the aa sequence of the corresponding CDRs of SEQ ID NO:169.

In certain embodiments, the anti-IGF-1R VHCDR1, VHCDR2, VHCDR3 of a PBA comprise the aa sequence of the corresponding CDRs of SEQ ID NO:10 and the anti-IGF-1R VLCDR1, VLCDR2 and VLCDR3 of the PBA comprise the aa sequence of the corresponding CDRs of SEQ ID NO:32; and (a) the anti-ErbB3 VHCDR1, VHCDR2, VHCDR3 of the PBA comprise the aa sequence of the corresponding CDRs of SEQ ID NO:134 and the anti-ErbB3 VLCDR1, VLCDR2 and VLCDR3 of the PBA comprise the aa sequence of the corresponding CDRs of SEQ ID NO: 166; or (b) the anti-ErbB3 VHCDR1, VHCDR2, VHCDR3 of the PBA comprise the aa sequence of the corresponding CDRs of SEQ ID NO:135 and the anti-ErbB3 VLCDR1, VLCDR2 and VLCDR3 of the PBA comprise the aa sequence of the corresponding CDRs of SEQ ID NO:167; or (c) the anti-ErbB3 VHCDR1, VHCDR2, VHCDR3 of the PBA comprise the aa sequence of the corresponding CDRs of SEQ ID NO: 136 and the anti-ErbB3 VLCDR1, VLCDR2 and VLCDR3 of the PBA comprise the aa sequence of the corresponding CDRs of SEQ ID NO: 168; or (d) the anti-ErbB3 VHCDR1, VHCDR2, VHCDR3 of the PBA comprise the aa sequence of the corresponding CDRs of SEQ ID NO:137 and the anti-ErbB3 VLCDR1, VLCDR2 and VLCDR3 of the PBA comprise the aa sequence of the corresponding CDRs of SEQ ID NO: 169; or (e) the anti-ErbB3 VHCDR1, VHCDR2, VHCDR3 of the PBA comprise the aa sequence of the corresponding CDRs of SEQ ID NO:138 and the anti-ErbB3 VLCDR1, VLCDR2 and VLCDR3 of the PBA comprise the aa sequence of the corresponding CDRs of SEQ ID NO: 170; or (f) the anti-ErbB3 VHCDR1, VHCDR2, VHCDR3 of the PBA comprise the aa sequence of the corresponding CDRs of SEQ ID NO:139 and the anti-ErbB3 VLCDR1, VLCDR2 and VLCDR3 of the PBA comprise the aa sequence of the corresponding CDRs of SEQ ID NO:171; or (g) the anti-ErbB3 VHCDR1, VHCDR2, VHCDR3 of the PBA comprise the aa sequence of the corresponding CDRs of SEQ ID NO: 140 and the anti-ErbB3 VLCDR1, VLCDR2 and VLCDR3 of the PBA comprise the aa sequence of the corresponding CDRs of SEQ ID NO: 172; or (h) the anti-ErbB3 VHCDR1, VHCDR2, VHCDR3 of the PBA comprise the aa sequence of the corresponding CDRs of SEQ ID NO: 141 and the anti-ErbB3 VLCDR1, VLCDR2 and VLCDR3 of the PBA comprise the aa sequence of the corresponding CDRs of SEQ ID NOs: 173; or (i) the anti-ErbB3 VHCDR1, VHCDR2, VHCDR3 of the PBA comprise the aa sequence of the corresponding CDRs of SEQ ID NO: 142 and the anti-ErbB3 VLCDR1, VLCDR2 and VLCDR3 of the PBA comprise the aa sequence of the corresponding CDRs of SEQ ID NO: 174; or (j) the anti-ErbB3 VHCDR1, VHCDR2, VHCDR3 of the PBA comprise the aa sequence of the corresponding CDRs of SEQ ID NO:143 and the anti-ErbB3 VLCDR1, VLCDR2 and VLCDR3 of the PBA comprise the aa sequence of the corresponding CDRs of SEQ ID NO:175; or (k) the anti-ErbB3 VHCDR1, VHCDR2, VHCDR3 of the PBA comprise the aa sequence of the corresponding CDRs of SEQ ID NO: 136 and the anti-ErbB3 VLCDR1, VLCDR2 and VLCDR3 of the PBA comprise the aa sequence of the corresponding CDRs of SEQ ID NO:169.

In certain embodiments, each anti-IGF-1R binding site of a PBA comprises a VH domain comprising the sequence of SEQ ID NO: 1, wherein the sequence comprises variable aas, and/or each anti-IGF-1R binding site of a PBA comprises a VL domain comprising the sequence of SEQ ID NO:2 (or 3), wherein the sequence comprises variable aas.

In certain embodiments, each anti-ErbB3 binding site of a PBA comprises a VH domain comprising the sequence of SEQ ID NO:4 (or 5), wherein the sequence comprises variable aas, and/or each anti-ErbB3 binding site of a PBA comprises a VL domain comprising the sequence of SEQ ID NO:6 (or 7), wherein the sequence comprises variable aas.

In certain embodiments, each anti-IGF-1R binding site of a PBA comprises a VH domain comprising the sequence of SEQ ID NO:1 and a VL domain comprising the sequence of SEQ ID NO:2 (or 3) and each anti-ErbB3 binding site of the PBA comprises a VH domain comprising the sequence of SEQ ID NO:4 (or 5) and a VL domain comprising the sequence of SEQ ID NO:6 (or 7).

In certain embodiments, each anti-IGF-1R binding site of a PBA comprises a VH domain comprising the aa sequence of SEQ ID NO:1, wherein X1 is not T, X2 is not V, X6 is not R, X8 is not D or X10 is not I, or a VL domain comprising the aa sequence of SEQ ID NO:3 or each anti-ErbB3 binding site of a PBA comprises a VH domain comprising the aa sequence of SEQ ID NO:5 or a VL domain comprising the sequence of SEQ ID NO:7. In certain embodiments, each anti-IGF-1R binding site of a PBA comprises a VH domain comprising the aa sequence of SEQ ID NO:1, wherein X1 is not T, X2 is not V, X6 is not R, X8 is not D or X10 is not I, a VL domain comprising the sequence of SEQ ID NO:3; and each anti-ErbB3 binding site of the PBA comprises a VH domain comprising the aa sequence of SEQ ID NO:5 and a VL domain comprising the sequence of SEQ ID NO:7.

In certain embodiments, each anti-IGF-1R binding site of a PBA comprises a VH domain comprising an aa sequence selected from the group consisting of SEQ ID NOs: 8-31 and/or a VL domain comprising an aa sequence selected from the group consisting of SEQ ID NOs: 32-133 and/or each anti-ErbB3 binding site comprises a VH aa sequence selected from the group consisting of SEQ ID NOs: 134-165; and/or a VL aa sequence selected from the group consisting of SEQ ID NOs: 166-200. In certain embodiments, (a) each first VH domain comprises an aa sequence selected from the group consisting of SEQ ID NOs: 8-31, each first VL domain comprises an aa sequence selected from the group consisting of SEQ ID NOs: 32-133, each second VH domain comprises an aa sequence selected from the group consisting of SEQ ID NOs: 134-165 and each second VL domain comprises an aa sequence selected from the group consisting of SEQ ID NOs: 166-200, or (b) each first VH domain comprises an aa sequence selected from the group consisting of SEQ ID NOs: 134-165, each first VL domain comprises an aa sequence selected from the group consisting of SEQ ID NOs: 166-200, each second VH domain comprises an aa sequence selected from the group consisting of SEQ ID NOs: 8-31 and each second VL domain consisting of an aa sequence selected from the group consisting of SEQ ID NOs: 32-133.

In certain embodiments, each anti-IGF-1R VH domain of a PBA comprises the aa sequence of SEQ ID NO:8 and each anti-IGF-1R VL domain of the PBA comprises the aa sequence of SEQ ID NO:32. In certain embodiments, each anti-IGF-1R VH domain of a PBA comprises the aa sequence of SEQ ID NO:9 and each anti-IGF-1R VL domain of the PBA comprises the aa sequence of SEQ ID NO:33. In certain embodiments, each anti-IGF-1R VH domain of a PBA comprises the aa sequence of SEQ ID NO:10 and each anti-IGF-1R VL domain of the PBA comprises the aa sequence of SEQ ID NO:34. In certain embodiments, each anti-IGF-1R VH domain of a PBA comprises the aa sequence of SEQ ID NO:11 and each anti-IGF-1R VL domain of the PBA comprises the aa sequence of SEQ ID NO:35. In certain embodiments, each anti-IGF-1R VH domain of a PBA comprises the aa sequence of SEQ ID NO:8 and each anti-IGF-1R VL domain of the PBA comprises the aa sequence of SEQ ID NO:33. In certain embodiments, each anti-IGF-1R VH domain of a PBA comprises the aa sequence of SEQ ID NO:10 and each anti-IGF-1R VL domain of the PBA comprises the aa sequence of SEQ ID NO:32.

In certain embodiments, each anti-ErbB3 VH domain of a PBA comprises the aa sequence of SEQ ID NO:134 and each anti-ErbB3 VL domain of the PBA comprises the aa sequence of SEQ ID NO:166. In certain embodiments, each anti-ErbB3 VH domain of a PBA comprises the aa sequence of SEQ ID NO: 135 and the anti-ErbB3 VL domain of the PBA comprise the aa sequence of SEQ ID NO:167. In certain embodiments, each anti-ErbB3 VH domain of a PBA comprises the aa sequence of SEQ ID NO:136 and each anti-ErbB3 VL domain of the PBA comprises the aa sequence of SEQ ID NO:168. In certain embodiments, each anti-ErbB3 VH domain of a PBA comprises the aa sequence of SEQ ID NO:137 and each anti-ErbB3 VL domain of the PBA comprises the aa sequence of SEQ ID NO:169. In certain embodiments, each anti-ErbB3 VH domain of a PBA comprises the aa sequence of SEQ ID NO:138 and each anti-ErbB3 VL domain of the PBA comprises the aa sequence of SEQ ID NO: 170. In certain embodiments, each anti-ErbB3 VH domain of a PBA comprises the aa sequence of SEQ ID NO:139 and each anti-ErbB3 VL domain of the PBA comprises the aa sequence of SEQ ID NO:171. In certain embodiments, each anti-ErbB3 VH domain of a PBA comprises the aa sequence of SEQ ID NO: 140 and each anti-ErbB3 VL domain of the PBA comprises the aa sequence of SEQ ID NO: 172. In certain embodiments, each anti-ErbB3 VH domain of a PBA comprises the aa sequence of SEQ ID NO: 141 and each anti-ErbB3 VL domain of the PBA comprises the aa sequence of SEQ ID NO: 173. In certain embodiments, each anti-ErbB3 VH domain of a PBA comprises the aa sequence of SEQ ID NO: 142 and each anti-ErbB3 VL domain of the PBA comprises the aa sequence of SEQ ID NO: 174. In certain embodiments, each anti-ErbB3 VH domain of a PBA comprises the aa sequence of SEQ ID NO: 143 and each anti-ErbB3 VL domain of the PBA comprises the aa sequence of SEQ ID NO: 175. In certain embodiments, each anti-ErbB3 VH domain of a PBA comprises the aa sequence of SEQ ID NO:136 and each anti-ErbB3 VL domain of the PBA comprises the aa sequence of SEQ ID NOs: 169.

In certain embodiments, each anti-IGF-1R VH domain of a PBA comprises the aa sequence of SEQ ID NO:8 and each anti-IGF-1R VL domain of the PBA comprises the aa sequence of SEQ ID NO:32; and (a) each anti-ErbB3 VH domain of the PBA comprises the aa sequence of SEQ ID NO: 134 and each anti-ErbB3 VL domain of the PBA comprises the aa sequence of SEQ ID NO: 166; or (b) each anti-ErbB3 VH domain of the PBA comprises the aa sequence of SEQ ID NO:135 and the anti-ErbB3 VL domain of the PBA comprise the aa sequence of SEQ ID NO: 167; or (c) each anti-ErbB3 VH domain of the PBA comprises the aa sequence of SEQ ID NO: 136 and each anti-ErbB3 VL domain of the PBA comprises the aa sequence of SEQ ID NO: 168; or (d) each anti-ErbB3 VH domain of the PBA comprises the aa sequence of SEQ ID NO: 137 and each anti-ErbB3 VL domain of the PBA comprises the aa sequence of SEQ ID NO: 169; or (e) each anti-ErbB3 VH domain of the PBA comprises the aa sequence of SEQ ID NO: 138 and each anti-ErbB3 VL domain of the PBA comprises the aa sequence of SEQ ID NO: 170; or (f) each anti-ErbB3 VH domain of the PBA comprises the aa sequence of SEQ ID NO: 139 and each anti-ErbB3 VL domain of the PBA comprises the aa sequence of SEQ ID NO:171; or (g) each anti-ErbB3 VH domain of the PBA comprises the aa sequence of SEQ ID NO: 140 and each anti-ErbB3 VL domain of the PBA comprises the aa sequence of SEQ ID NO: 172; or (h) each anti-ErbB3 VH domain of the PBA comprises the aa sequence of SEQ ID NO: 141 and each anti-ErbB3 VL domain of the PBA comprises the aa sequence of SEQ ID NO: 173; or (i) each anti-ErbB3 VH domain of the PBA comprises the aa sequence of SEQ ID NO: 142 and each anti-ErbB3 VL domain of the PBA comprises the aa sequence of SEQ ID NO: 174; or (j) each anti-ErbB3 VH domain of the PBA comprises the aa sequence of SEQ ID NO: 143 and each anti-ErbB3 VL domain of the PBA comprises the aa sequence of SEQ ID NO: 175; or (k) each anti-ErbB3 VH domain of the PBA comprises the aa sequence of SEQ ID NO: 136 and each anti-ErbB3 VL domain of the PBA comprises the aa sequence of SEQ ID NOs: 169.

In certain embodiments, each anti-IGF-1R VH domain of a PBA comprises the aa sequence of SEQ ID NO:9 and each anti-IGF-1R VL domain of the PBA comprises the aa sequence of SEQ ID NO:33; and (a) each anti-ErbB3 VH domain of the PBA comprises the aa sequence of SEQ ID NO: 134 and each anti-ErbB3 VL domain of the PBA comprises the aa sequence of SEQ ID NO: 166; or (b) each anti-ErbB3 VH domain of the PBA comprises the aa sequence of SEQ ID NO:135 and the anti-ErbB3 VL domain of the PBA comprise the aa sequence of SEQ ID NO: 167; or (c) each anti-ErbB3 VH domain of the PBA comprises the aa sequence of SEQ ID NO: 136 and each anti-ErbB3 VL domain of the PBA comprises the aa sequence of SEQ ID NO: 168; or (d) each anti-ErbB3 VH domain of the PBA comprises the aa sequence of SEQ ID NO: 137 and each anti-ErbB3 VL domain of the PBA comprises the aa sequence of SEQ ID NO: 169; or (e) each anti-ErbB3 VH domain of the PBA comprises the aa sequence of SEQ ID NO: 138 and each anti-ErbB3 VL domain of the PBA comprises the aa sequence of SEQ ID NO: 170; or (f) each anti-ErbB3 VH domain of the PBA comprises the aa sequence of SEQ ID NO: 139 and each anti-ErbB3 VL domain of the PBA comprises the aa sequence of SEQ ID NO:171; or (g) each anti-ErbB3 VH domain of the PBA comprises the aa sequence of SEQ ID NO: 140 and each anti-ErbB3 VL domain of the PBA comprises the aa sequence of SEQ ID NO: 172; or (h) each anti-ErbB3 VH domain of the PBA comprises the aa sequence of SEQ ID NO: 141 and each anti-ErbB3 VL domain of the PBA comprises the aa sequence of SEQ ID NO: 173; or (i) each anti-ErbB3 VH domain of the PBA comprises the aa sequence of SEQ ID NO: 142 and each anti-ErbB3 VL domain of the PBA comprises the aa sequence of SEQ ID NO: 174; or (j) each anti-ErbB3 VH domain of the PBA comprises the aa sequence of SEQ ID NO: 143 and each anti-ErbB3 VL domain of the PBA comprises the aa sequence of SEQ ID NO: 175; or (k) each anti-ErbB3 VH domain of the PBA comprises the aa sequence of SEQ ID NO: 136 and each anti-ErbB3 VL domain of the PBA comprises the aa sequence of SEQ ID NOs: 169.

In certain embodiments, each anti-IGF-1R VH domain comprises the aa sequence of SEQ ID NO:10 and each anti-IGF-1R VL domain comprises the aa sequence of SEQ ID NO:34; and (a) each anti-ErbB3 VH domain of the PBA comprises the aa sequence of SEQ ID NO:134 and each anti-ErbB3 VL domain of the PBA comprises the aa sequence of SEQ ID NO: 166; or (b) each anti-ErbB3 VH domain of the PBA comprises the aa sequence of SEQ ID NO: 135 and the anti-ErbB3 VL domain of the PBA comprise the aa sequence of SEQ ID NO: 167; or (c) each anti-ErbB3 VH domain of the PBA comprises the aa sequence of SEQ ID NO: 136 and each anti-ErbB3 VL domain of the PBA comprises the aa sequence of SEQ ID NO: 168; or (d) each anti-ErbB3 VH domain of the PBA comprises the aa sequence of SEQ ID NO: 137 and each anti-ErbB3 VL domain of the PBA comprises the aa sequence of SEQ ID NO: 169; or (e) each anti-ErbB3 VH domain of the PBA comprises the aa sequence of SEQ ID NO:138 and each anti-ErbB3 VL domain of the PBA comprises the aa sequence of SEQ ID NO: 170; or (f) each anti-ErbB3 VH domain of the PBA comprises the aa sequence of SEQ ID NO:139 and each anti-ErbB3 VL domain of the PBA comprises the aa sequence of SEQ ID NO:171; or (g) each anti-ErbB3 VH domain of the PBA comprises the aa sequence of SEQ ID NO: 140 and each anti-ErbB3 VL domain of the PBA comprises the aa sequence of SEQ ID NO: 172; or (h) each anti-ErbB3 VH domain of the PBA comprises the aa sequence of SEQ ID NO: 141 and each anti-ErbB3 VL domain of the PBA comprises the aa sequence of SEQ ID NO: 173; or (i) each anti-ErbB3 VH domain of the PBA comprises the aa sequence of SEQ ID NO: 142 and each anti-ErbB3 VL domain of the PBA comprises the aa sequence of SEQ ID NO: 174; or (j) each anti-ErbB3 VH domain of the PBA comprises the aa sequence of SEQ ID NO: 143 and each anti-ErbB3 VL domain of the PBA comprises the aa sequence of SEQ ID NO: 175; or (k) each anti-ErbB3 VH domain of the PBA comprises the aa sequence of SEQ ID NO: 136 and each anti-ErbB3 VL domain of the PBA comprises the aa sequence of SEQ ID NOs: 169.

In certain embodiments, each anti-IGF-1R VH domain of a PBA comprises the aa sequence of SEQ ID NO:11 and each anti-IGF-1R VL domain of the PBA comprises the aa sequence of SEQ ID NO:35; and (a) each anti-ErbB3 VH domain of the PBA comprises the aa sequence of SEQ ID NO: 134 and each anti-ErbB3 VL domain of the PBA comprises the aa sequence of SEQ ID NO: 166; or (b) each anti-ErbB3 VH domain of the PBA comprises the aa sequence of SEQ ID NO:135 and the anti-ErbB3 VL domain of the PBA comprise the aa sequence of SEQ ID NO: 167; or (c) each anti-ErbB3 VH domain of the PBA comprises the aa sequence of SEQ ID NO: 136 and each anti-ErbB3 VL domain of the PBA comprises the aa sequence of SEQ ID NO: 168; or (d) each anti-ErbB3 VH domain of the PBA comprises the aa sequence of SEQ ID NO: 137 and each anti-ErbB3 VL domain of the PBA comprises the aa sequence of SEQ ID NO: 169; or (e) each anti-ErbB3 VH domain of the PBA comprises the aa sequence of SEQ ID NO: 138 and each anti-ErbB3 VL domain of the PBA comprises the aa sequence of SEQ ID NO: 170; or (f) each anti-ErbB3 VH domain of the PBA comprises the aa sequence of SEQ ID NO: 139 and each anti-ErbB3 VL domain of the PBA comprises the aa sequence of SEQ ID NO:171; or (g) each anti-ErbB3 VH domain of the PBA comprises the aa sequence of SEQ ID NO: 140 and each anti-ErbB3 VL domain of the PBA comprises the aa sequence of SEQ ID NO: 172; or (h) each anti-ErbB3 VH domain of the PBA comprises the aa sequence of SEQ ID NO: 141 and each anti-ErbB3 VL domain of the PBA comprises the aa sequence of SEQ ID NO: 173; or (i) each anti-ErbB3 VH domain of the PBA comprises the aa sequence of SEQ ID NO: 142 and each anti-ErbB3 VL domain of the PBA comprises the aa sequence of SEQ ID NO: 174; or (j) each anti-ErbB3 VH domain of the PBA comprises the aa sequence of SEQ ID NO: 136 and each anti-ErbB3 VL domain of the PBA comprises the aa sequence of SEQ ID NOs: 169.

In certain embodiments, each anti-IGF-1R VH domain of the PBA comprises the aa sequence of SEQ ID NO:8 and each anti-IGF-1R VL domain of the PBA comprises the aa sequence of SEQ ID NO:33; and (a) each anti-ErbB3 VH domain of the PBA comprises the aa sequence of SEQ ID NO: 134 and each anti-ErbB3 VL domain of the PBA comprises the aa sequence of SEQ ID NO: 166; or (b) each anti-ErbB3 VH domain of the PBA comprises the aa sequence of SEQ ID NO:135 and the anti-ErbB3 VL domain of the PBA comprise the aa sequence of SEQ ID NO: 167; or (c) each anti-ErbB3 VH domain of the PBA comprises the aa sequence of SEQ ID NO: 136 and each anti-ErbB3 VL domain of the PBA comprises the aa sequence of SEQ ID NO: 168; or (d) each anti-ErbB3 VH domain of the PBA comprises the aa sequence of SEQ ID NO: 137 and each anti-ErbB3 VL domain of the PBA comprises the aa sequence of SEQ ID NO: 169; or (e) each anti-ErbB3 VH domain of the PBA comprises the aa sequence of SEQ ID NO: 138 and each anti-ErbB3 VL domain of the PBA comprises the aa sequence of SEQ ID NO: 170; or (f) each anti-ErbB3 VH domain of the PBA comprises the aa sequence of SEQ ID NO: 139 and each anti-ErbB3 VL domain of the PBA comprises the aa sequence of SEQ ID NO:171; or (g) each anti-ErbB3 VH domain of the PBA comprises the aa sequence of SEQ ID NO: 140 and each anti-ErbB3 VL domain of the PBA comprises the aa sequence of SEQ ID NO: 172; or (h) each anti-ErbB3 VH domain of the PBA comprises the aa sequence of SEQ ID NO: 141 and each anti-ErbB3 VL domain of the PBA comprises the aa sequence of SEQ ID NO: 173; or (i) each anti-ErbB3 VH domain of the PBA comprises the aa sequence of SEQ ID NO: 142 and each anti-ErbB3 VL domain of the PBA comprises the aa sequence of SEQ ID NO: 174; or (j) each anti-ErbB3 VH domain of the PBA comprises the aa sequence of SEQ ID NO: 143 and each anti-ErbB3 VL domain of the PBA comprises the aa sequence of SEQ ID NO: 175; or (k) each anti-ErbB3 VH domain of the PBA comprises the aa sequence of SEQ ID NO: 136 and each anti-ErbB3 VL domain of the PBA comprises the aa sequence of SEQ ID NOs: 169.

In certain embodiments, each anti-IGF-1R VH domain of a PBA comprises the aa sequence of SEQ ID NO:10 and each anti-IGF-1R VL domain of the PBA comprises the aa sequence of SEQ ID NO:32; and (a) each anti-ErbB3 VH domain of the PBA comprises the aa sequence of SEQ ID NO: 134 and each anti-ErbB3 VL domain of the PBA comprises the aa sequence of SEQ ID NO: 166; or (b) each anti-ErbB3 VH domain of the PBA comprises the aa sequence of SEQ ID NO:135 and the anti-ErbB3 VL domain of the PBA comprise the aa sequence of SEQ ID NO: 167; or (c) each anti-ErbB3 VH domain of the PBA comprises the aa sequence of SEQ ID NO: 136 and each anti-ErbB3 VL domain of the PBA comprises the aa sequence of SEQ ID NO: 168; or (d) each anti-ErbB3 VH domain of the PBA comprises the aa sequence of SEQ ID NO: 137 and each anti-ErbB3 VL domain of the PBA comprises the aa sequence of SEQ ID NO: 169; or (e) each anti-ErbB3 VH domain of the PBA comprises the aa sequence of SEQ ID NO: 138 and each anti-ErbB3 VL domain of the PBA comprises the aa sequence of SEQ ID NO: 170; or (f) each anti-ErbB3 VH domain of the PBA comprises the aa sequence of SEQ ID NO: 139 and each anti-ErbB3 VL domain of the PBA comprises the aa sequence of SEQ ID NO:171; or (g) each anti-ErbB3 VH domain of the PBA comprises the aa sequence of SEQ ID NO: 140 and each anti-ErbB3 VL domain of the PBA comprises the aa sequence of SEQ ID NO: 172; or (h) each anti-ErbB3 VH domain of the PBA comprises the aa sequence of SEQ ID NO: 141 and each anti-ErbB3 VL domain of the PBA comprises the aa sequence of SEQ ID NO: 173; or (i) each anti-ErbB3 VH domain of the PBA comprises the aa sequence of SEQ ID NO: 142 and each anti-ErbB3 VL domain of the PBA comprises the aa sequence of SEQ ID NO: 174; or (j) each anti-ErbB3 VH domain of the PBA comprises the aa sequence of SEQ ID NO: 143 and each anti-ErbB3 VL domain of the PBA comprises the aa sequence of SEQ ID NO: 175; or (k) each anti-ErbB3 VH domain of the PBA comprises the aa sequence of SEQ ID NO: 136 and each anti-ErbB3 VL domain of the PBA comprises the aa sequence of SEQ ID NOs: 169.

In certain embodiments, (a) each heavy chain of a PBA comprises an aa sequence selected from the group consisting of SF-G1-P1 (SEQ ID NO:212); SF-G1-M1.3 (SEQ ID NO:214); SF-G1-M27 (SEQ ID NO:216); SF-G1-P6 (SEQ ID NO:218); SF-G1-B69 (SEQ ID NO:220); P4-G1-C8 (SEQ ID NO:222); P4-G1-P1 (SEQ ID NO:224); P4-G1-M1.3 (SEQ ID NO:226); P4-G1-M27 (SEQ ID NO:228); P4-G1-P6 (SEQ ID NO:230); P4-G1-B69 (SEQ ID NO:232); M78-G1-C8 (SEQ ID NO:234); M78-G1-P1 (SEQ ID NO:236); M78-G1-M1.3 (SEQ ID NO:238); M78-G1-M27 (SEQ ID NO:240); M78-G1-P6 (SEQ ID NO:242); M78-G1-B69 (SEQ ID NO:244); M57-G1-C8 (SEQ ID NO:246); M57-G1-P1 (SEQ ID NO:248); M57-G1-M1.3 (SEQ ID NO:250); M57-G1-M27 (SEQ ID NO:252); M57-G1-P6 (SEQ ID NO:254) and M57-G1-B69 (SEQ ID NO:256) and/or each light chain of the PBA comprises an aa sequence selected from the group consisting of SF kappa light chain (SEQ ID NO:202); P4 kappa light chain (SEQ ID NO:204); M78 kappa light chain (SEQ ID NO:206); and M57 kappa light chain (SEQ ID NO:208); or (b) each heavy chain of a PBA comprises an aa sequence selected from the group consisting of P1-G1-P4 (SEQ ID NO:268); P1-G1-M57 (SEQ ID NO:270); P1-G1-M78 (SEQ ID NO:272); M27-G1-P4 (SEQ ID NO:274); M27-G1-M57 (SEQ ID NO:276); M27-G1-M78 (SEQ ID NO:278); M7-G1-P4 (SEQ ID NO:280); M7-G1-M57 ((SEQ ID NO:282); M7-G1-M78 (SEQ ID NO:284); B72-G1-P4 (SEQ ID NO:286); B72-G1-M57 (SEQ ID NO:288); B72-G1-M78 (SEQ ID NO:290); B60-G1-P4 (SEQ ID NO:292); B60-G1-M57 (SEQ ID NO:294); B60-G1-M78 (SEQ ID NO:296); B60-G2-M78 (SEQ ID NO:355) and M7-G2-M78 (SEQ ID NO:357) and/or each light chain of the PBA comprises an aa sequence selected from the group consisting of P1 lambda light chain (SEQ ID NO:258); M27 lambda light chain (SEQ ID NO:260); M7 lambda light chain (SEQ ID NO:262); B72 lambda light chain (SEQ ID NO:264); and B60 lambda light chain (SEQ ID NO:266).

In certain embodiments, (a) each heavy chain of a PBA comprises an aa sequence differing in at least one aa addition, deletion or substitution from an aa sequence selected from the group consisting of SF-G1-P1 (SEQ ID NO:212); SF-G1-M1.3 (SEQ ID NO:214); SF-G1-M27 (SEQ ID NO:216); SF-G1-P6 (SEQ ID NO:218); SF-G1-B69 (SEQ ID NO:220); P4-G1-C8 (SEQ ID NO:222); P4-G1-P1 (SEQ ID NO:224); P4-G1-M1.3 (SEQ ID NO:226); P4-G1-M27 (SEQ ID NO:228); P4-G1-P6 (SEQ ID NO:230); P4-G1-B69 (SEQ ID NO:232); M78-G1-C8 (SEQ ID NO:234); M78-G1-P1 (SEQ ID NO:236); M78-G1-M1.3 (SEQ ID NO:238); M78-G1-M27 (SEQ ID NO:240); M78-G1-P6 (SEQ ID NO:242); M78-G1-B69 (SEQ ID NO:244); M57-G1-C8 (SEQ ID NO:246); M57-G1-P1 (SEQ ID NO:248); M57-G1-M1.3 (SEQ ID NO:250); M57-G1-M27 (SEQ ID NO:252); M57-G1-P6 (SEQ ID NO:254) and M57-G1-B69 (SEQ ID NO:256) and each light chain of the PBA comprises an aa sequence selected from the group consisting of SF kappa light chain (SEQ ID NO:202); P4 kappa light chain (SEQ ID NO:204); M78 kappa light chain (SEQ ID NO:206); and M57 kappa light chain (SEQ ID NO:208); or (b) each heavy chain of a PBA comprises an aa sequence selected from the group consisting of SF-G1-P1 (SEQ ID NO:212); SF-G1-M1.3 (SEQ ID NO:214); SF-G1-M27 (SEQ ID NO:216); SF-G1-P6 (SEQ ID NO:218); SF-G1-B69 (SEQ ID NO:220); P4-G1-C8 (SEQ ID NO:222); P4-G1-P1 (SEQ ID NO:224); P4-G1-M1.3 (SEQ ID NO:226); P4-G1-M27 (SEQ ID NO:228); P4-G1-P6 (SEQ ID NO:230); P4-G1-B69 (SEQ ID NO:232); M78-G1-C8 (SEQ ID NO:234); M78-G1-P1 (SEQ ID NO:236); M78-G1-M1.3 (SEQ ID NO:238); M78-G1-M27 (SEQ ID NO:240); M78-G1-P6 (SEQ ID NO:242); M78-G1-B69 (SEQ ID NO:244); M57-G1-C8 (SEQ ID NO:246); M57-G1-P1 (SEQ ID NO:248); M57-G1-M1.3 (SEQ ID NO:250); M57-G1-M27 (SEQ ID NO:252); M57-G1-P6 (SEQ ID NO:254) and M57-G1-B69 (SEQ ID NO:256); and each light chain of the PBA comprises an aa sequence differing in at least one aa addition, deletion or substitution from an aa sequence selected from the group consisting of SF kappa light chain (SEQ ID NO:202); P4 kappa light chain (SEQ ID NO:204); M78 kappa light chain (SEQ ID NO:206); and M57 kappa light chain (SEQ ID NO:208); or (c) each heavy chain of a PBA comprises an aa sequence differing in at least one aa addition, deletion or substitution from an aa sequence selected from the group consisting of P1-G1-P4 (SEQ ID NO:268); P1-G1-M57 (SEQ ID NO:270); P1-G1-M78 (SEQ ID NO:272); M27-G1-P4 (SEQ ID NO:274); M27-G1-M57 (SEQ ID NO:276); M27-G1-M78 (SEQ ID NO:278); M7-G1-P4 (SEQ ID NO:280); M7-G1-M57 ((SEQ ID NO:282); M7-G1-M78 (SEQ ID NO:284); B72-G1-P4 (SEQ ID NO:286); B72-G1-M57 (SEQ ID NO:288); B72-G1-M78 (SEQ ID NO:290); B60-G1-P4 (SEQ ID NO:292); B60-G1-M57 (SEQ ID NO:294); B60-G1-M78 (SEQ ID NO:296); B60-G2-M78 (SEQ ID NO:355) and M7-G2-M78 (SEQ ID NO:357) and each light chain of the PBA comprises an aa sequence selected from the group consisting of P1 lambda light chain (SEQ ID NO:258); M27 lambda light chain (SEQ ID NO:260); M7 lambda light chain (SEQ ID NO:262); B72 lambda light chain (SEQ ID NO:264); and B60 lambda light chain (SEQ ID NO:266); or (d) each heavy chain of a PBA comprises an aa sequence selected from the group consisting of P1-G1-P4 (SEQ ID NO:268); P1-G1-M57 (SEQ ID NO:270); P1-G1-M78 (SEQ ID NO:272); M27-G1-P4 (SEQ ID NO:274); M27-G1-M57 (SEQ ID NO:276); M27-G1-M78 (SEQ ID NO:278); M7-G1-P4 (SEQ ID NO:280); M7-G1-M57 ((SEQ ID NO:282); M7-G1-M78 (SEQ ID NO:284); B72-G1-P4 (SEQ ID NO:286); B72-G1-M57 (SEQ ID NO:288); B72-G1-M78 (SEQ ID NO:290); B60-G1-P4 (SEQ ID NO:292); B60-G1-M57 (SEQ ID NO:294); B60-G1-M78 (SEQ ID NO:296); B60-G2-M78 (SEQ ID NO:355) and M7-G2-M78 (SEQ ID NO:357) and each light chain of the PBA comprises an aa sequence differing in at least one aa addition, deletion or substitution from an aa sequence selected from the group consisting of P1 lambda light chain (SEQ ID NO:258); M27 lambda light chain (SEQ ID NO:260); M7 lambda light chain (SEQ ID NO:262); B72 lambda light chain (SEQ ID NO:264); and B60 lambda light chain (SEQ ID NO:266), wherein the PBA differs from 16F in at least one aa, CDR or variable domain.

In certain embodiments, (a) each heavy chain of a PBA, bound to the other heavy chain of the PBA by at least one bond, comprises an aa sequence that is at least 90% identical to one of the following aa sequences or differs from one of the following aa sequences in 1-30 aa substitutions, deletions and/or additions: SF-G1-P1 (SEQ ID NO:212); SF-G1-M1.3 (SEQ ID NO:214); SF-G1-M27 (SEQ ID NO:216); SF-G1-P6 (SEQ ID NO:218); SF-G1-B69 (SEQ ID NO:220); P4-G1-C8 (SEQ ID NO:222); P4-G1-P1 (SEQ ID NO:224); P4-G1-M1.3 (SEQ ID NO:226); P4-G1-M27 (SEQ ID NO:228); P4-G1-P6 (SEQ ID NO:230); P4-G1-B69 (SEQ ID NO:232); M78-G1-C8 (SEQ ID NO:234); M78-G1-P1 (SEQ ID NO:236); M78-G1-M1.3 (SEQ ID NO:238); M78-G1-M27 (SEQ ID NO:240); M78-G1-P6 (SEQ ID NO:242); M78-G1-B69 (SEQ ID NO:244); M57-G1-C8 (SEQ ID NO:246); M57-G1-P1 (SEQ ID NO:248); M57-G1-M1.3 (SEQ ID NO:250); M57-G1-M27 (SEQ ID NO:252); M57-G1-P6 (SEQ ID NO:254) and M57-G1-B69 (SEQ ID NO:256) and (b) each light chain of the PBA, bound to one heavy chain of (a) by at least one bond, comprises an aa sequence that is at least 90% identical to one of the following aa sequences or differs from one of the following aa sequences in 1-30 aa substitutions, deletions and/or additions: SF kappa light chain (SEQ ID NO:202); P4 kappa light chain (SEQ ID NO:204); M78 kappa light chain (SEQ ID NO:206); and M57 kappa light chain (SEQ ID NO:208); or (c) each heavy chain of a PBA, bound to the other heavy chain of the PBA by at least one bond, comprises an aa sequence that is at least 90% identical to one of the following aa sequences or differs from one of the following aa sequences in 1-30 aa substitutions, deletions and/or additions: P1-G1-P4 (SEQ ID NO:268); P1-G1-M57 (SEQ ID NO:270); P1-G1-M78 (SEQ ID NO:272); M27-G1-P4 (SEQ ID NO:274); M27-G1-M57 (SEQ ID NO:276); M27-G1-M78 (SEQ ID NO:278); M7-G1-P4 (SEQ ID NO:280); M7-G1-M57 ((SEQ ID NO:282); M7-G1-M78 (SEQ ID NO:284); B72-G1-P4 (SEQ ID NO:286); B72-G1-M57 (SEQ ID NO:288); B72-G1-M78 (SEQ ID NO:290); B60-G1-P4 (SEQ ID NO:292); B60-G1-M57 (SEQ ID NO:294); and B60-G1-M78 (SEQ ID NO:296)); B60-G2-M78 (SEQ ID NO:355) and M7-G2-M78 (SEQ ID NO:357) and (d) each light chain of the PBA, bound to one heavy chain of (c) by at least one bond, comprises an aa sequence that is at least 90% identical to one of the following aa sequences or which differs from one of the following aa sequences in 1-30 aa substitutions, deletions and/or additions: P1 lambda light chain (SEQ ID NO:258); M27 lambda light chain (SEQ ID NO:260); M7 lambda light chain (SEQ ID NO:262); B72 lambda light chain (SEQ ID NO:264); and B60 lambda light chain (SEQ ID NO:266).

In certain embodiments, (a) each heavy chain of a PBA comprises an aa sequence that is at least 95% identical to one of the following aa sequence or differs from one of the following aa sequences in 1-10 aa substitutions, deletions and/or additions: SF-G1-P1 (SEQ ID NO:212); SF-G1-M1.3 (SEQ ID NO:214); SF-G1-M27 (SEQ ID NO:216); SF-G1-P6 (SEQ ID NO:218); SF-G1-B69 (SEQ ID NO:220); P4-G1-C8 (SEQ ID NO:222); P4-G1-P1 (SEQ ID NO:224); P4-G1-M1.3 (SEQ ID NO:226); P4-G1-M27 (SEQ ID NO:228); P4-G1-P6 (SEQ ID NO:230); P4-G1-B69 (SEQ ID NO:232); M78-G1-C8 (SEQ ID NO:234); M78-G1-P1 (SEQ ID NO:236); M78-G1-M1.3 (SEQ ID NO:238); M78-G1-M27 (SEQ ID NO:240); M78-G1-P6 (SEQ ID NO:242); M78-G1-B69 (SEQ ID NO:244); M57-G1-C8 (SEQ ID NO:246); M57-G1-P1 (SEQ ID NO:248); M57-G1-M1.3 (SEQ ID NO:250); M57-G1-M27 (SEQ ID NO:252); M57-G1-P6 (SEQ ID NO:254) and M57-G1-B69 (SEQ ID NO:256), and (b) each light chain of the PBA comprises an aa sequence that is at least 95% identical to one of the following aa sequences or differs from one of the following aa sequences in 1-10 aa substitutions, deletions and/or additions: SF kappa light chain (SEQ ID NO:202); P4 kappa light chain (SEQ ID NO:204); M78 kappa light chain (SEQ ID NO:206); and M57 kappa light chain (SEQ ID NO:208); or (c) each heavy chain of a PBA comprises an aa sequence that is at least 95% identical to one of the following aa sequences or differs from one of the following aa sequences in 1-10 aa substitutions, deletions and/or additions: P1-G1-P4 (SEQ ID NO:268); P1-G1-M57 (SEQ ID NO:270); P1-G1-M78 (SEQ ID NO:272); M27-G1-P4 (SEQ ID NO:274); M27-G1-M57 (SEQ ID NO:276); M27-G1-M78 (SEQ ID NO:278); M7-G1-P4 (SEQ ID NO:280); M7-G1-M57 ((SEQ ID NO:282); M7-G1-M78 (SEQ ID NO:284); B72-G1-P4 (SEQ ID NO:286); B72-G1-M57 (SEQ ID NO:288); B72-G1-M78 (SEQ ID NO:290); B60-G1-P4 (SEQ ID NO:292); B60-G1-M57 (SEQ ID NO:294); and B60-G1-M78 (SEQ ID NO:296); B60-G2-M78 (SEQ ID NO:355) and M7-G2-M78 (SEQ ID NO:357) and (d) each light chain of the PBA comprises an aa sequence that is at least 95% identical to one of the following aa sequences or differs from one of the following aa sequences in 1-10 aa substitutions, deletions and/or additions: P1 lambda light chain (SEQ ID NO:258); M27 lambda light chain (SEQ ID NO:260); M7 lambda light chain (SEQ ID NO:262); B72 lambda light chain (SEQ ID NO:264); B60 lambda light chain (SEQ ID NO:266).

Exemplary PBA include the following: (a) an SF-G1-P1 PBA comprising: two heavy chains, each comprising a heavy chain aa sequence of SEQ ID NO:212; and two light chains, each comprising a light chain sequence of SEQ ID NO:202; (b) An SF-G1-M1.3 PBA comprising: two heavy chains, each comprising a heavy chain aa sequence of SEQ ID NO:214; and two light chains, each comprising a light chain aa sequence of SEQ ID NO:202; (c) an SF-G1-M27 PBA comprising: two heavy chains, each comprising a heavy chain aa sequence of SEQ ID NO:216; and two light chains, each comprising a light chain aa sequence of SEQ ID NO:202; (d) an SF-G1-P6 PBA comprising: two heavy chains, each comprising a heavy chain aa sequence of SEQ ID NO:218; and two light chains, each comprising a light chain aa sequence of SEQ ID NO:202; (e) an SF-G1-B69 PBA comprising: two heavy chains, each comprising a heavy chain aa sequence of SEQ ID NO:220; and two light chains, each comprising a light chain aa sequence of SEQ ID NO:202; (f) a P4-G1-C8 PBA comprising: two heavy chains, each comprising a heavy chain aa sequence of SEQ ID NO:222; and two light chains, each comprising a light chain aa sequence of SEQ ID NO:204 (g) a P4-G1-P1 PBA comprising: two heavy chains, each comprising a heavy chain aa sequence of SEQ ID NO:224; and two light chains, each comprising a light chain aa sequence of SEQ ID NO:204; (h) a P4-G1-M1.3 PBA comprising: two heavy chains, each comprising a heavy chain aa sequence of SEQ ID NO:226; and two light chains, each comprising a light chain aa sequence of SEQ ID NO:204; (i) a P4-G1-M27 PBA comprising: two heavy chains, each comprising a heavy chain aa sequence of SEQ ID NO:228; and two light chains, each comprising a light chain aa sequence of SEQ ID NO:204; (j) a P4-G1-P6 PBA comprising: two heavy chains, each comprising a heavy chain aa sequence of SEQ ID NO:230; and two light chains, each comprising a light chain aa sequence of SEQ ID NO:204; (h) a P4-G1-B69 PBA comprising: two heavy chains, each comprising a heavy chain aa sequence of SEQ ID NO:232; and two light chains, each comprising a light chain aa sequence of SEQ ID NO:204; (i) an M78-G1-C8 PBA comprising: two heavy chains, each comprising a heavy chain aa sequence of SEQ ID NO:234; and two light chains, each comprising a light chain aa sequence of SEQ ID NO:206; (j) an M78-G1-P1 PBA comprising: two heavy chains, each comprising a heavy chain aa sequence of SEQ ID NO:236; and two light chains, each comprising a light chain aa sequence of SEQ ID NO:206; (k) an M78-G1-M1.3 PBA comprising: two heavy chains, each comprising a heavy chain aa sequence of SEQ ID NO:238; and two light chains, each comprising a light chain aa sequence of SEQ ID NO:206; (l) an M78-G1-M27 PBA comprising: two heavy chains, each comprising a heavy chain aa sequence of SEQ ID NO:240; and two light chains, each comprising a light chain aa sequence of SEQ ID NO:206; (m) an M78-G1-P6 PBA comprising: two heavy chains, each comprising a heavy chain aa sequence of SEQ ID NO:242; and two light chains, each comprising a light chain aa sequence of SEQ ID NO:206; (n) an M78-G1-B69 PBA comprising: two heavy chains, each comprising a heavy chain aa sequence of SEQ ID NO:244; and two light chains, each comprising a light chain aa sequence of SEQ ID NO:206; (o) an M57-G1-C8 PBA comprising: two heavy chains, each comprising a heavy chain aa sequence of SEQ ID NO:246; and two light chains, each comprising a light chain aa sequence of SEQ ID NO:208; (p) an M57-G1-P1 PBA comprising: two heavy chains, each comprising a heavy chain aa sequence of SEQ ID NO:248; and two light chains, each comprising a light chain aa sequence of SEQ ID NO:208; (r) an M57-G1-M1.3 PBA comprising: two heavy chains, each comprising a heavy chain aa sequence of SEQ ID NO:250; and two light chains, each comprising a light chain aa sequence of SEQ ID NO:208; (s) an M57-G1-M27 PBA comprising: two heavy chains, each comprising a heavy chain aa sequence of SEQ ID NO:252; and two light chains, each comprising a light chain aa sequence of SEQ ID NO:208; (t) an M57-G1-P6 PBA comprising: two heavy chains, each comprising a heavy chain aa sequence of SEQ ID NO:254; and two light chains, each comprising a light chain aa sequence of SEQ ID NO:208; (u) an M57-G1-B69 PBA comprising: two heavy chains, each comprising a heavy chain aa sequence of SEQ ID NO:256; and two light chains, each comprising a light chain aa sequence of SEQ ID NO:208; (v) a P1-G1-P4 PBA comprising: two heavy chains, each comprising a heavy chain aa sequence of SEQ ID NO:268; and two light chains, each comprising a light chain aa sequence of SEQ ID NO:258; (w) a P1-G1-M57 PBA comprising: two heavy chains, each comprising a heavy chain aa sequence of SEQ ID NO:270; and two light chains, each comprising a light chain aa sequence of SEQ ID NO:258; (x) a P1-G1-M78 PBA comprising: two heavy chains, each comprising a heavy chain aa sequence of SEQ ID NO:272; and two light chains, each comprising a light chain aa sequence of SEQ ID NO:258; (y) an M27-G1-P4 PBA comprising: two heavy chains, each comprising a heavy chain aa sequence of SEQ ID NO:274; and two light chains, each comprising a light chain aa sequence of SEQ ID NO:260; (z) an M27-G1-M57 PBA comprising: two heavy chains, each comprising a heavy chain aa sequence of SEQ ID NO:276; and two light chains, each comprising a light chain aa sequence of SEQ ID NO:260; (aa) an M27-G1-M78 PBA comprising: two heavy chains, each comprising a heavy chain aa sequence of SEQ ID NO:278; and two light chains, each comprising a light chain aa sequence of SEQ ID NO:260; (ab) an M7-G1-P4 PBA comprising: two heavy chains, each comprising a heavy chain aa sequence of SEQ ID NO:280; and two light chains, each comprising a light chain aa sequence of SEQ ID NO:262; (ac) an M7-G1-M57 PBA comprising: two heavy chains, each comprising a heavy chain aa sequence of SEQ ID NO:282; and two light chains, each comprising a light chain aa sequence of SEQ ID NO:262; (ad) an M7-G1-M78 PBA comprising: two heavy chains, each comprising a heavy chain aa sequence of SEQ ID NO:284; and two light chains, each comprising a light chain aa sequence of SEQ ID NO:262; (ae) a B72-G1-P4 PBA comprising: two heavy chains, each comprising a heavy chain aa sequence of SEQ ID NO:286; and two light chains, each comprising a light chain aa sequence of SEQ ID NO:264; (af) a B72-G1-M57 PBA comprising: two heavy chains, each comprising a heavy chain aa sequence of SEQ ID NO:288; and two light chains, each comprising a light chain aa sequence of SEQ ID NO:264; (ag) a B72-G1-M78 PBA comprising: two heavy chains, each comprising a heavy chain aa sequence of SEQ ID NO:290; and two light chains, each comprising a light chain aa sequence of SEQ ID NO:264; (ah) a B60-G1-P4 PBA comprising: two heavy chains, each comprising a heavy chain aa sequence of SEQ ID NO:292; and two light chains, each comprising a light chain aa sequence of SEQ ID NO:266; (ai) a B60-G1-M57 PBA comprising: two heavy chains, each comprising a heavy chain aa sequence of SEQ ID NO:294; and two light chains, each comprising a light chain aa sequence of SEQ ID NO:266; and (aj) a B60-G1-M78 PBA comprising: two heavy chains, each comprising a heavy chain aa sequence of SEQ ID NO:296; and two light chains, each comprising a light chain aa sequence of SEQ ID NO:266.

Pharmaceutical Compositions

Compounds useful in the methods of this disclosure can be administered as pharmaceutical compositions by any conventional route, in particular enterally, e.g., orally, e.g., in the form of tablets or capsules, or parenterally, e.g., in the form of injectable solutions or suspensions, topically, e.g., in the form of lotions, gels, ointments or creams, or in a nasal or suppository form. Pharmaceutical compositions comprising a compound useful in the disclosed methods in free form or in a pharmaceutically acceptable salt form in association with at least one pharmaceutically acceptable carrier or diluent can be manufactured in a conventional manner by mixing, granulating or coating methods. For example, oral compositions can be tablets or gelatin capsules comprising the active ingredient together with a) diluents, e.g., lactose, dextrose, sucrose, mannitol, sorbitol, cellulose and/or glycine; b) lubricants, e.g., silica, talcum, stearic acid, its magnesium or calcium salt and/or polyethyleneglycol; for tablets also c) binders, e.g., magnesium aluminum silicate, starch paste, gelatin, tragacanth, methylcellulose, sodium carboxymethylcellulose and or polyvinylpyrrolidone; if desired d) disintegrants, e.g., starches, agar, alginic acid or its sodium salt, or effervescent mixtures; and/or e) absorbents, colorants, flavors and sweeteners. Injectable compositions can be aqueous isotonic solutions or suspensions, and suppositories can be prepared from fatty emulsions or suspensions. The compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Suitable formulations for transdermal applications include an effective amount of a compound with a carrier. A carrier can include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin. Matrix transdermal formulations may also be used. Suitable formulations for topical application, e.g., to the skin and eyes, are preferably aqueous solutions, ointments, creams or gels well-known in the art. Such may contain solubilizers, stabilizers, tonicity enhancing agents, buffers and preservatives.

Compounds useful in the disclosed methods can be administered in therapeutically effective amounts in combination with one or more therapeutic agents (pharmaceutical combinations). Where the compounds are administered in conjunction with other therapies, dosages of the co-administered compounds will of course vary depending on the type of co-drug employed, on the specific drug employed, on the condition being treated and so forth.

Combination therapy includes administration to the subject compounds in further combination with other biologically active ingredients (such as, but not limited to, a second and different antineoplastic agent) and non-drug therapies (such as, but not limited to, surgery or radiation treatment). For instance, the compounds can be used in combination with other pharmaceutically active compounds, preferably compounds that are able to enhance the effect of the original pharmaceutical combination. The compounds can be administered simultaneously (as a single preparation or separate preparation) or sequentially to the other drug therapy. In general, a combination therapy envisions administration of two or more drugs during a single cycle or course of therapy.

In certain embodiments, these compositions optionally further comprise one or more additional therapeutic agents. Alternatively, a compound useful in the disclosed methods may be administered to a patient in need thereof in combination with the administration of one or more other therapeutic agents. For example, additional therapeutic agents for conjoint administration or inclusion in a pharmaceutical composition with a compound useful in the disclosed methods are an agent that impedes regulatory T-cell (Treg) activity. The second agent may be selected from, but is not limited to, a small molecule immunomodulatory agent, an immunomodulatory antibody that does not bind human IGF-1, an anticancer vaccine, and a radiation dose.

It will also be appreciated that the compounds and pharmaceutical compositions useful in the disclosed methods can be used in combination therapies, that is, the compounds and pharmaceutical compositions can be administered concurrently with, prior to, or subsequent to, one or more other desired therapeutics or medical procedures. The particular combination of therapies (therapeutics or procedures) to use in a combination regimen will take into account compatibility of the desired therapeutics and/or procedures and the desired therapeutic effect to be achieved. It will also be appreciated that the therapies used may achieve a desired effect for the same disorder (for example, a compound may be administered concurrently with another anticancer agent), or they may achieve different effects (e.g., control of any adverse effects).

For example, other therapies or anticancer agents that may be used in combination with the compounds useful in the disclosed methods include, but are not limited to, surgery, radiotherapy (e.g., gamma-radiation, neutron beam radiotherapy, electron beam radiotherapy, proton therapy, brachytherapy, and systemic radioactive isotopes), endocrine therapy, biologic response modifiers (e.g., interferons, interleukins, antibodies, aptamers, siRNAs, oligonucleotides, enzyme, ion channel and receptor inhibitors or activators), hyperthermia and cryotherapy, agents to attenuate any adverse effects (e.g., antiemetics), and other approved chemotherapeutic drugs, including, but not limited to, alkylating drugs (e.g., mechlorethamine, chlorambucil, cyclophosphamide, melphalan HCl, ifosfamide), antimetabolites (e.g., methotrexate), purine antagonists and pyrimidine antagonists (e.g., 6-mercaptopurine, 5-fluorouracil, cytarabine (cytosine arabinoside), gemcitabine), spindle poisons (e.g., vinblastine, vincristine, vinorelbine, paclitaxel, nab-paclitaxel), podophyllotoxins (e.g., etoposide, irinotecan, topotecan), antibiotics (e.g., doxorubicin, bleomycin, mitomycin), nitrosoureas (e.g., carmustine, lomustine), inorganic ions (e.g., cisplatin, carboplatin), enzymes (e.g., asparaginase), and hormones (e.g., tamoxifen, leuprolide, flutamide, and megestrol). For a more comprehensive discussion of updated cancer therapies see The Merck Manual, Seventeenth Ed. 1999, the entire contents of which are hereby incorporated by reference. See also the National Cancer Institute (CNI) website (nci.nih.gov) and the Food and Drug Administration (FDA) website for a list of the FDA approved oncology drugs (fda.gov/cder/cancer/dmglistfrarne).

In certain embodiments, the pharmaceutical compositions useful in the disclosed methods further comprise one or more additional therapeutically active ingredients (e.g., chemotherapeutic and/or palliative). For purposes of this disclosure, the term “palliative” refers to treatment that is focused on the relief of symptoms of a disease and/or side effects of a therapeutic regimen, but is not curative. For example, palliative treatment encompasses painkillers, anti-nausea medications, anti-pyretics, and anti-sickness drugs. In addition, chemotherapy, radiotherapy and surgery can all be used palliatively (that is, to reduce symptoms without going for cure; e.g., for shrinking tumors and reducing pressure, bleeding, pain and other symptoms of cancer).

The compounds and compositions can be administered together with hormonal and steroidal anti-inflammatory agents, such as but not limited to, estradiol, conjugated estrogens (e.g., PREMARIN, PREMPRO, AND PREMPHASE), 17 beta estradiol, calcitonin-salmon, levothyroxine, dexamethasone, medroxyprogesterone, prednisone, cortisone, flunisolide, and hydrocortisone; non-steroidal anti-inflammatory agents, such as but not limited to, tramadol, fentanyl, metamizole, ketoprofen, naproxen, nabumetone, ketoralac, tromethamine, loxoprofen, ibuprofen, aspirin, and acetaminophen; anti-TNFalpha antibodies, such as infliximab (REMICADE) and etanercept (ENBREL).

The pharmaceutical compositions useful in the disclosed methods comprise a therapeutically effective amount of a compound formulated together with one or more pharmaceutically acceptable carriers. As used herein, the term “pharmaceutically acceptable carrier” means a non-toxic, inert solid, semi-solid or liquid filler, diluent, encapsulating material or formulation auxiliary of any type. The pharmaceutical compositions useful in the methods disclosed herein can be administered to humans and other animals orally, rectally, parenterally, intracisternally, intravaginally, intraperitoneally, topically (as by powders, ointments, or drops), buccally, or as an oral or nasal spray.

According to the disclosed methods of treatment, disorders are treated or prevented in a subject, such as a human patient or other animal, by administering to the subject a therapeutically effective amount of a compound, in such amounts and for such time as is necessary to achieve the desired result.

In general, compounds useful in the disclosed methods are administered in therapeutically effective amounts via any of the usual and acceptable modes known in the art, either singly or in combination with one or more therapeutic agents. A therapeutically effective amount may vary widely depending on the severity of the disease, the age and relative health of the subject, the potency of the compound used and other factors. Therapeutic amounts or doses also vary depending on route of administration, as well as the possibility of co-usage with other agents.

Dosages

In some embodiments, the bispecific antibody is administered at one of the following schedules: 6 mg/kg qw, 9 mg/kg qw, 8 mg/kg qw, 10 mg/kg q2w, 12 mg/kg q3w, 18 mg/kg q3w, 20 mg/kg q3w, 30 mg/kg q3w, 20 mg/kg qw, or 40 mg/kg q2w. Alternatively, the bispecific antibody is administered according to the dose schedules outlined in Table 2.

Biological Samples

The disclosed methods may involve taking a biological sample from a patient in order to determine the gene mutation status of the sample or the gene expression level status of the sample. For example, the biological sample may be a from a tumor tissue, tumor biopsy, whole blood, blood serum, blood plasma, semen, urine, mucus, or other body sample. In a preferred embodiment, the biological sample is blood serum, blood plasma, tumor tissue, or tumor biopsy sample. Tumor tissue may be formalin-fixed paraffin embedded tumor tissue or fresh frozen tumor tissue.

Biomarkers

Methods of detecting many useful biomarkers are well known. Some such useful methods are disclosed in the EXAMPLES below and others may be found in WIPO Publication No. WO 2015/100459; U.S. Pat. No. 8,623,592; WIPO Publication No. WO/2016/054414, and U.S. patent application Ser. No. 14/967,158, each of which is incorporated by reference in its entirety. Biomarkers useful for the present disclosure include, but are not limited to, those biomarkers that are used in the recognition of, for diagnosis of, or for determining the prognosis of one or more of the following disorders: cancer, myeloma, pancreatic cancer, ovarian cancer, hepatocellular carcinoma, parathyroid cancer, sarcoma, lung cancer, colon carcinoma, B-cell lymphoma, and breast cancer. Biomarker levels may be assessed using a biological sample by any method known in the art, which may include but is not limited to RT-PCR (reverse transcription polymerase chain reaction), PCR-ISH (polymerase chain reaction in situ hybridization), and RNA-ISH (RNA in situ hybridization). For example, the patient to be evaluated for using the methods disclosed herein may be selected for treatment by assessing a biomarker level in a biopsy of the cancer and the patient may then treated with the bispecific antibody if the biopsy exhibits a level that is predictive of a favorable response to the treatment. In various embodiments of this aspect, the biomarker is 1) fraction of IGF-1R positive regulatory T cells, 2) fraction of IGF-1R positive cytotoxic T cells, or 3) the ratio of 1) to 2); and the level that is predictive of a favorable response to the treatment is where 1) or 2) or 3) is higher than the fraction or ratio corresponding to a population median level of patients with the cancer. In other embodiments, the biomarker is serum free IGF-1 and the level that is predictive of a favorable response to the treatment is where the patient's serum free IGF-1 concentration is higher than a population median level of patients with the cancer. In yet other embodiments of this aspect, the biomarker is biopsy heregulin level and the level that is predictive of a favorable response to the treatment is where the biopsy heregulin level is higher than a population median biopsy heregulin level of patients with the cancer; or the biomarker is biopsy heregulin level and the level that is predictive of a favorable response to the treatment is a biopsy heregulin level that is sufficient to be detectable by RT-PCR; or the biomarker is biopsy heregulin level and the level that is predictive of a favorable response to the treatment is a biopsy heregulin level that sufficient to be detectable by RNA-ISH.

Kits

Also provided herein are kits for use in the disclosed methods. In one embodiment, the kit comprises a first agent that is a bispecific anti-IGF-1R/anti-ErbB3 antibody that immunospecifically binds to human ErbB3 and to the extracellular domain of human IGF-1R (CD221), a second agent that impedes regulatory T cell activity, and instructions for use. The second agent may be selected from one or more of: a small molecule immunomodulatory agent, an immunomodulatory antibody that does not bind human IGF-1, and an anticancer vaccine. The immunomodulatory antibody of the kit may be one or more antibody selected from the group consisting of (a) an agonistic anti-receptor antibody that immunospecifically binds human OX40, CD40, GITR, CD27, ICOS, or 4-1BB; (b) an antagonistic anti-receptor antibody that immunospecifically binds human CTLA-4, PD-1, PD-L1, TIM-3, BTLA, VISTA, LAG-3, KIR, CD47, CD25, B7-H3, or B7-H4; and (c) an anti-ligand antibody that blocks function of IL-6, IL-10, TGFβ, angiopoetin-2, VEGF, IL-17, IL-23, or TNFα. In one embodiment, the anti-CTLA-4 antibody is ipilimumab or tremelimumab. In another embodiment, the anti-PD-1 antibody is selected from the group consisting of nivolumab, pembrolizumab, and pidilizumab. In another embodiment, the anti-PD-L1 antibody is selected from the group consisting of atezolizumab, durvalumab, and avelumab.

The anti-KIR antibody may be lirilumab. The anticancer vaccine may be selected from the group consisting of OncoVex, MAGE-A3, PROSTVAC, GVAX, CDX110, CDX1307, CDX1401, CV9104, BIOVAXID, IMA 901 and ADXS11-001.

### Illustrative Additional Embodiments

Non-limiting examples of certain embodiments are provided below:
1. A method of treating cancer in a patient, comprising administering to the patient an effective amount of a first agent that is a bispecific anti-IGF-1R/anti-ErbB3 antibody that immunospecifically binds to human ErbB3 and to the extracellular domain of human IGF-1R (CD221), and a second agent that impedes regulatory T cell activity, wherein the cancer is treated.
2. The method of embodiment 1, wherein the first agent binding to IGF-1R inhibits IGF-1-mediated activation of human IGF-1R.
3. The method of embodiment 1 or 2, wherein the effective amount of the first agent exerts at least one effect selected from the group consisting of (a) inhibiting proliferation of IGF-1R ligand-dependent and/or IGF-1R ligand-independent IGF-1R expressing immune cells; (b) inhibiting differentiation of IGF-1R expressing immune progenitor cells into T cells, NKT cells, or NK cells; and (c) depleting IGF-1R expressing immune cells.
4. The method of any one of embodiments 1-3, wherein the second agent is selected from the group consisting of a small molecule immunomodulatory agent, an immunomodulatory antibody that does not bind human IGF1, an anticancer vaccine, and a radiation dose.
5. The method of any one of embodiments 1-3, wherein the immunomodulatory antibody is at least one selected from the group consisting of (a) an agonistic anti-receptor antibody that immunospecifically binds human OX40, CD40, GITR, CD27, ICOS, or 4-1BB; (b) an antagonistic anti-receptor antibody that immunospecifically binds human CTLA-4, PD-1, PD-L1, TIM-3, BTLA, VISTA, LAG-3, KIR, CD47, CD25, B7-H3, or B7-H4; and (c) an anti-ligand antibody that blocks function of IL-6, IL-10, TGFβ, angiopoetin-2, VEGF, IL-17, IL-23, or TNFα.
6. The method of embodiment 5, wherein the anti-CTLA-4 antibody is ipilimumab or tremelimumab.
7. The method of embodiment 5, wherein the anti-PD-1 antibody is selected from the group consisting of nivolumab, pembrolizumab, and pidilizumab.
8. The method of embodiment 5, wherein the anti-PD-L1 antibody is selected from the group consisting of atezolizumab, durvalumab, and avelumab.
9. The method of embodiment 5, wherein the anti-KIR antibody is lirilumab.
10. The method of embodiment 4, wherein the anticancer vaccine is selected from the group consisting of OncoVex, MAGE-A3, PROSTVAC, GVAX, CDX110, CDX1307, CDX1401, CV9104, BIOVAXID, IMA 901 and ADXS11-001.
11. The method of embodiment 1, wherein the first agent is selected from the group consisting of istiratumab (P4-G1-M1.3), SF-G1-P1, SF-G1-M1.3, SF-G1-M27, SF-G1-P6, SF-G1-B69, P4-G1-C8, P4-G1-P1, P4-G1-M27, P4-G1-P6, P4-G1-B69, M78-G1-C8, M78-G1-P1, M78-G1-M1.3, M78-G1-M27, M78-G1-P6, M78-G1-B69, M57-G1-C8, M57-G1-P1, M57-G1-M1.3, M57-G1-M27, M57-G1-P6, M57-G1-B69, P1-G1-P4, P1-G1-M57, P1-G1-M78, M27-G1-P4, M27-G1-M57, M27-G1-M78, M7-G1-P4, M7-G1-M57, M7-G1-M78, B72-G1-P4, B72-G1-M57, B72-G1-M78, B60-G1-P4, B60-G1-M57, B60-G1-M78, P4M-G1-M1.3, P4M-G1-C8, P33M-G1-M1.3, P33M-G1-C8, P4M-G1-P6L, P33M-G1-P6L, and P1-G1-M76.
12. The method of any of embodiments 1-12, wherein the first agent is administered intravenously or subcutaneously.
13. The method of any of embodiments 1-13, wherein the first agent is administered intravenously at 6 mg/kg once weekly, 9 mg/kg once weekly, 8 mg/kg once weekly, 10 mg/kg every two weeks, 12 mg/kg every three weeks, 18 mg/kg every three weeks, 20 mg/kg every three weeks, 30 mg/kg every three weeks, 20 mg/kg once weekly, or 40 mg/kg every two weeks.
14. The method of embodiment 1, wherein the first agent has been engineered to increase or decrease Fc receptor binding.
15. The method of any of the preceding embodiments, wherein the first agent preferentially binds to regulatory T cells compared to CD8 positive (CD8+) cytotoxic T cells.
16. The method of embodiment 15, wherein the regulatory T cells are CD4 positive (CD4+) and either or both of CD25 positive (CD25+) and FoxP3 positive (FoxP3+).
17. The method of any of the preceding embodiments, wherein the patient is selected by assessing a biomarker level in a biopsy of the cancer, and the patient is treated if the biopsy exhibits a level that is predictive of treatment responsiveness.
18. The method of embodiment 17, wherein the biomarker is (a) fraction of IGF-1R positive regulatory T cells, (b) fraction of IGF-1R positive cytotoxic T cells, or (c) a ratio of (a) to (b); and a level that is predictive of treatment responsiveness is where (a) or (b) or (c) is higher than the fraction (a), fraction (b) or ratio of (a) to (b) corresponding to a population median level of patients with the cancer.
19. The method of embodiment 17 or 18, wherein the biomarker is serum free IGF-1 (i.e., free IGF-1 from the patient's serum), and the level that is predictive of treatment responsiveness is where the patient's serum free IGF-1 concentration is higher than a population median level of patients with the cancer.
20. The method of any one of embodiments 17-19, wherein the biomarker is biopsy heregulin level, and the level that is predictive of treatment responsiveness is where the biopsy heregulin level is higher than a population median biopsy heregulin level of patients with the cancer.
21. The method of any one of embodiments 17-20, wherein the biomarker is biopsy heregulin level, and the level that is predictive of treatment responsiveness is a biopsy heregulin level that is sufficient to be detectable by RT-PCR.
22. The method of any one of embodiments 17-20, wherein the biomarker is biopsy heregulin level, and the level that is predictive of treatment responsiveness is a biopsy heregulin level that is sufficient to be detectable by RNA-ISH.
23. The method of any of the preceding embodiments, wherein the cancer is selected from the group consisting of myeloma, pancreatic cancer, ovarian cancer, hepatocellular carcinoma, parathyroid cancer, sarcoma, lung cancer, colon carcinoma, B-cell lymphoma, and breast cancer.
24. The method of embodiment 1, wherein the first agent is istiratumab, the second agent is an anti-PD-L1 antibody, and the cancer is selected from the group consisting of pancreatic cancer, ovarian cancer, hepatocellular carcinoma, parathyroid cancer, sarcoma, lung cancer, colon carcinoma, B cell lymphoma, and breast cancer.
25. The method of embodiment 24, wherein the anti-PD-L1 antibody is atezolizumab (MPDL3280a), and the cancer is colon carcinoma.
26. The method of embodiment 1, wherein the first agent is istiratumab, the second agent is an anti-CTLA-4 antibody, and the cancer is melanoma.
27. The method of any of the preceding embodiments, wherein administration of the first agent and second agent in combination more effectively treats the cancer than when each of the first and second agent is administered as monotherapy at a same amount as the combination.
28. A method of treating a cancer in a patient comprising administering an effective amount of a bispecific anti-IGF-1R/anti-ErbB3 antibody that immunospecifically binds to human ErbB3 and to the extracellular domain of human IGF-1R (CD221) to the patient, wherein the cancer is treated, and wherein the cancer is selected from the group consisting of pancreatic cancer, ovarian cancer, hepatocellular carcinoma, parathyroid cancer, sarcoma, lung cancer and breast cancer.
29. The method of embodiment 28, wherein the administering of the bispecific antibody correlates with reduced expression of IGF-1R on splenocyte-derived regulatory T cells, but not in CD8+ T cells.
30. The method of embodiment 28, wherein the administering of the bispecific antibody correlates with reduced proliferation of regulatory T cells.
31. The method of any of embodiments 28-30 wherein the bispecific antibody is selected from the group consisting of istiratumab (P4-G1-M1.3), SF-G1-P1, SF-G1-M1.3, SF-G1-M27, SF-G1-P6, SF-G1-B69, P4-G1-C8, P4-G1-P1, P4-G1-M27, P4-G1-P6, P4-G1-B69, M78-G1-C8, M78-G1-P1, M78-G1-M1.3, M78-G1-M27, M78-G1-P6, M78-G1-B69, M57-G1-C8, M57-G1-P1, M57-G1-M1.3, M57-G1-M27, M57-G1-P6, M57-G1-B69, P1-G1-P4, P1-G1-M57, P1-G1-M78, M27-G1-P4, M27-G1-M57, M27-G1-M78, M7-G1-P4, M7-G1-M57, M7-G1-M78, B72-G1-P4, B72-G1-M57, B72-G1-M78, B60-G1-P4, B60-G1-M57, B60-G1-M78, P4M-G1-M1.3, P4M-G1-C8, P33M-G1-M1.3, P33M-G1-C8, P4M-G1-P6L, P33M-G1-P6L, and P1-G1-M76.
32. The method of embodiment 31, wherein the bispecific antibody is istiratumab.
33. A method of recruiting antitumor activity of innate immune cells (non-T cells) and T cells to a tumor in a patient, comprising administering to the patient an effective amount of a bispecific anti-IGF-1R/anti-ErbB3 antibody that immunospecifically binds to human ErbB3 and to the extracellular domain of human IGF-1R (CD221) to the patient, wherein antitumor activity of innate immune cells and T cells is recruited to the tumor.
34. The method of embodiment 33, wherein the tumor does not express IGFR-1 or ErbB3 receptors.
35. The method of embodiment 33 or 34, wherein the tumor is a B cell lymphoma tumor.
36. The method of any of embodiments 33-35, wherein the bispecific antibody is selected from the group consisting of istiratumab (P4-G1-M1.3), SF-G1-P1, SF-G1-M1.3, SF-G1-M27, SF-G1-P6, SF-G1-B69, P4-G1-C8, P4-G1-P1, P4-G1-M27, P4-G1-P6, P4-G1-B69, M78-G1-C8, M78-G1-P1, M78-G1-M1.3, M78-G1-M27, M78-G1-P6, M78-G1-B69, M57-G1-C8, M57-G1-P1, M57-G1-M1.3, M57-G1-M27, M57-G1-P6, M57-G1-B69, P1-G1-P4, P1-G1-M57, P1-G1-M78, M27-G1-P4, M27-G1-M57, M27-G1-M78, M7-G1-P4, M7-G1-M57, M7-G1-M78, B72-G1-P4, B72-G1-M57, B72-G1-M78, B60-G1-P4, B60-G1-M57, B60-G1-M78, P4M-G1-M1.3, P4M-G1-C8, P33M-G1-M1.3, P33M-G1-C8, P4M-G1-P6L, P33M-G1-P6L, and P1-G1-M76.
37. The method of any one of embodiments 33-36, wherein the bispecific antibody is istiratumab.
38. A kit, comprising a first agent that is a bispecific anti-IGF-1R/anti-ErbB3 antibody that immunospecifically binds to human ErbB3 and to the extracellular domain of human IGF-1R (CD221), a second agent that impedes regulatory T cell activity, and instructions for use.
39. The kit of embodiment 38, wherein the first agent is selected from the group consisting of istiratumab (P4-G1-M1.3), SF-G1-P1, SF-G1-M1.3, SF-G1-M27, SF-G1-P6, SF-G1-B69, P4-G1-C8, P4-G1-P1, P4-G1-M27, P4-G1-P6, P4-G1-B69, M78-G1-C8, M78-G1-P1, M78-G1-M1.3, M78-G1-M27, M78-G1-P6, M78-G1-B69, M57-G1-C8, M57-G1-P1, M57-G1-M1.3, M57-G1-M27, M57-G1-P6, M57-G1-B69, P1-G1-P4, P1-G1-M57, P1-G1-M78, M27-G1-P4, M27-G1-M57, M27-G1-M78, M7-G1-P4, M7-G1-M57, M7-G1-M78, B72-G1-P4, B72-G1-M57, B72-G1-M78, B60-G1-P4, B60-G1-M57, B60-G1-M78, P4M-G1-M1.3, P4M-G1-C8, P33M-G1-M1.3, P33M-G1-C8, P4M-G1-P6L, P33M-G1-P6L, and P1-G1-M76.
40. The kit of embodiment 39, wherein the second agent is selected from the group consisting of a small molecule immunomodulatory agent, an immunomodulatory antibody that does not bind human IGF1, and an anticancer vaccine.
41. The kit of embodiment 40, wherein the immunomodulatory antibody is at least one selected from the group consisting of (a) an agonistic anti-receptor antibody that immunospecifically binds human OX40, CD40, GITR, CD27, ICOS, or 4-1BB; (b) an antagonistic anti-receptor antibody that immunospecifically binds human CTLA-4, PD-1, PD-L1, TIM-3, BTLA, VISTA, LAG-3, KIR, CD47, CD25, B7-H3, or B7-H4; and (c) an anti-ligand antibody that blocks function of IL-6, IL-10, TGFβ, angiopoetin-2, VEGF, IL-17, IL-23, or TNFα.
42. The kit of embodiment 41, wherein the anti-CTLA-4 antibody is ipilimumab or tremelimumab.
43. The kit of embodiment 41, wherein the anti-PD-1 antibody is selected from the group consisting of nivolumab, pembrolizumab, and pidilizumab.
44. The kit of embodiment 41, wherein the anti-PD-L1 antibody is selected from the group consisting of atezolizumab, durvalumab, and avelumab.
45. The kit of embodiment 41, wherein the anti-KIR antibody is lirilumab.
46. The kit of embodiment 40, wherein the anticancer vaccine is selected from the group consisting of OncoVex, MAGE-A3, PROSTVAC, GVAX, CDX110, CDX1307, CDX1401, CV9104, BIOVAXID, IMA 901 and ADXS11-001.

### EXAMPLES

The present disclosure is further illustrated by the following examples which should not be construed as further limiting. The contents of Sequence Listing, figures and all references, patents, and published patent applications cited throughout this application are expressly incorporated herein by reference.

### Example 1

This Example describes the results of treatment of CD4+ T cells with insulin-like growth factor ligand (IGF-1), wherein the treatment of CD4+ T cells with IGF-1 produces a greater percentage of regulatory T cells from CD4+ T cells (Tregs; here identified as CD4+, CD25+, FoxP3+ cells by flow cytometry) and also increases the proliferation of these Tregs in vitro.

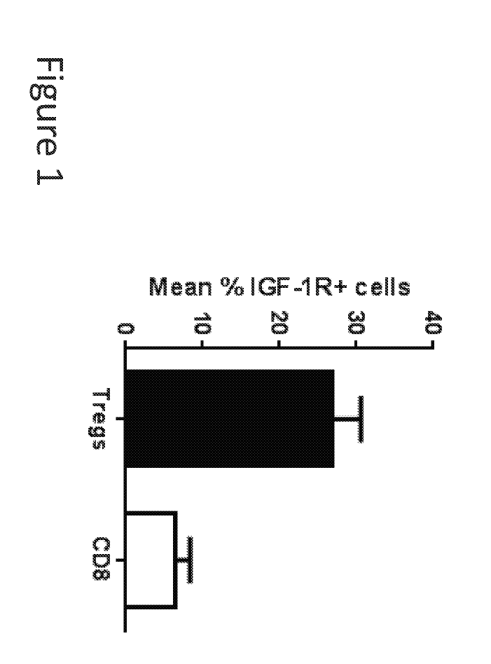

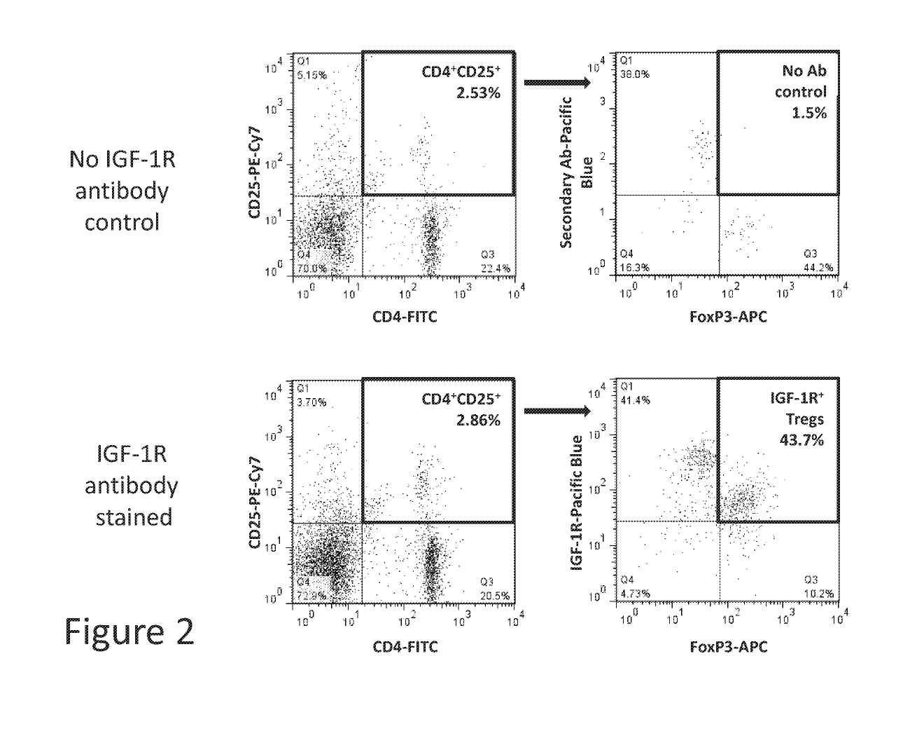

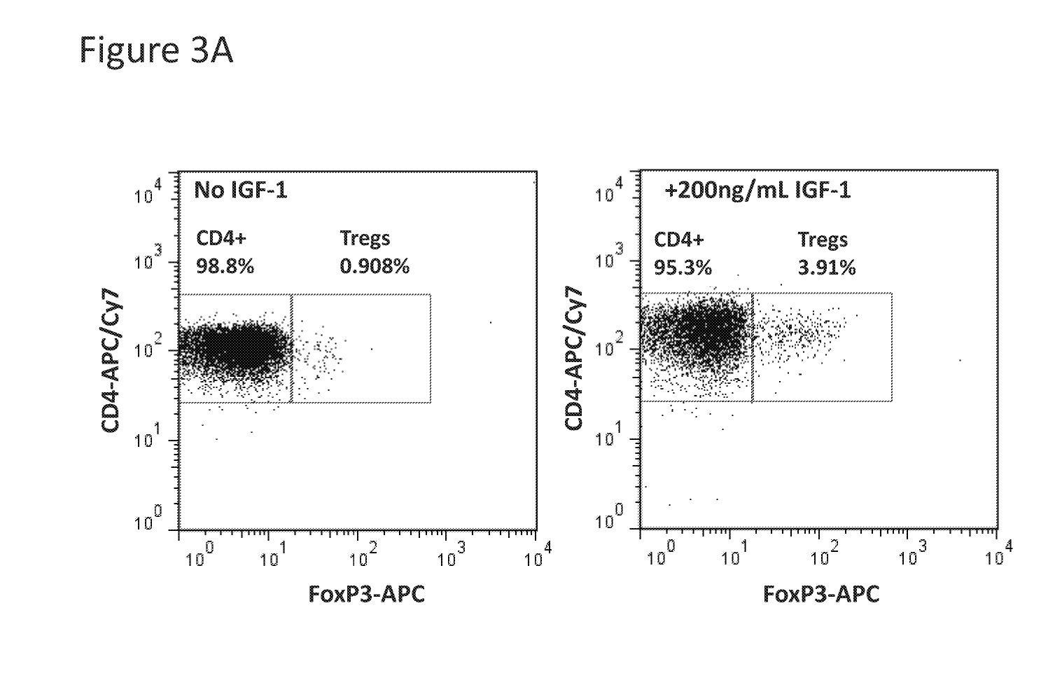

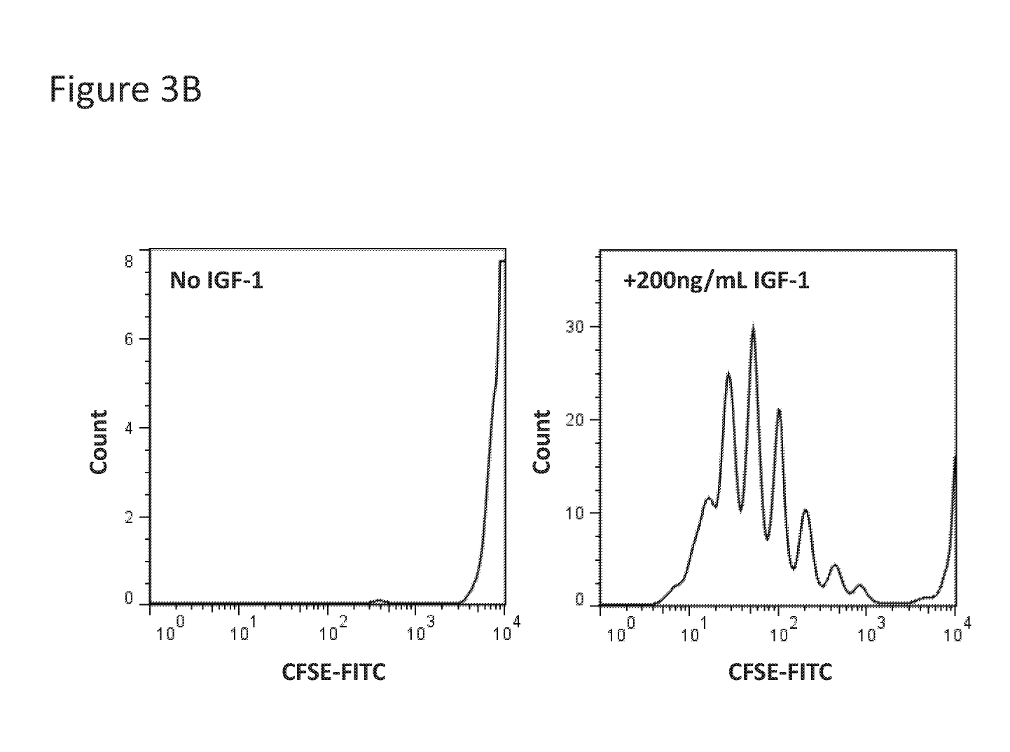

The regulatory T cells (Tregs), formerly known as suppressor T cells, are a subpopulation of T cells which modulate the immune system, suppress immune responses against other cells and maintain tolerance to self-antigens. As used herein, Tregs are defined as CD4+ and either or both of CD25+ and FoxP3+. Tregs are also CD8 negative (CD8-). Tregs and CD8+ T cells (cytotoxic T cells) derived from whole blood from a disease-free human patient express IGF-1R, with greater expression of this receptor on Treg cells compared to CD8+ T cells (FIG. 1). Female C57BL/6 (Charles River Labs) mouse-derived splenocytes also express IGF-1R on the surface of their Tregs (FIG. 2). In addition, treatment of mouse splenocyte-derived CD4+ T cells with IGF-1 led to increased induction of Tregs in vitro. The percentage of Treg cells increased from 0.908% to 3.91% of CD4+ cells treated with 0 and 200 ng/mL IGF-1, respectively (FIG. 3A). Moreover, analyses of proliferation measured by CFSE dilution indicated enhanced proliferation of these Treg populations (FIG. 3B), as indicated by the multiple peaks present in cells treated with IGF-1, which are indicative of cell division. CFSE is a cell-permeant fluorescein-based dye that covalently attaches to cytoplasmic components of cells, resulting in uniform bright fluorescence. Upon cell division, the dye is distributed equally between daughter cells, allowing the resolution of up to eight cycles of cell division by flow cytometry.

For the quantitative flow cytometry experiments outlined in FIG. 1, human whole blood was collected from disease free patients in non-EDTA containing K2 Vacutainers® and maintained at room temperature until analysis (<24 hours). The white blood cell fraction was isolated from the whole blood by centrifugation over Ficoll-Paque™ (GE Healthcare: Life Sciences) and red blood cells lysed by incubation in ACK buffer (Life Technologies) for 2 minutes. Total cell counts were obtained and aliquots of cells were stained with CD25-PerCp/Cy5.5, CD4-APC/Cy7, CD8-AmCyan, FoxP3-APC, IGF-1R-biotin and streptavidin-Brilliant Violet™ 421. Percent expression of IGF-1R+ Tregs or CD8+ cells was determined by analyzing the percentage of Tregs (CD4+, CD25+, FoxP3+) or CD8+ cells expressing IGF-1R as measured by quantitative flow cytometry (FACS Canto, BD Biosciences). After the acquisition, all gates and data analyses were performed using the FlowJo® 8.7 software (FlowJo, LLC).

For the quantitative flow cytometry experiments outlined in FIG. 2, spleens were extracted from C57BL/6 mice, homogenized to single cell suspension, and red blood cells lysed by incubation in ACK (Ammonium-Chloride-Potassium) Lysing Buffer (Life Technologies) for 2 minutes. Total cell counts were obtained and aliquots of cells were stained with CD25-Pe/Cy7, CD4-FITC, FoxP3-APC, IGF-1R-biotin and streptavidin-Brilliant Violet™ 421, which is visible in the Pacific Blue channel. All fluorescent antibodies were sourced from BioLegend®. To confirm IGF-1R specific staining, an aliquot of cells was stained with streptavidin-Brilliant Violet™ 421 but without IGF-1R-biotin and used to set the gates for calculations. Percent expression of IGF-1R+ Tregs was determined by analyzing the percentage of Tregs (here identified as CD4+, CD25+, and FoxP3+) expressing IGF-1R as measured by quantitative flow cytometry (FACS Canto, BD Biosciences). After the acquisition, all gates and data analyses were performed using the FlowJo 8.7 software.

For the induction and proliferation of Tregs in vitro as outlined in FIG. 3, CD4+ T cells were isolated from C57BL/6 mouse spleens as outlined in FIG. 2. These cells were incubated with CFSE (5 μM) for 8 minutes at 37° C., washed and plated at 40,000 cells per well in a 96 well plate in media containing TGF-beta (0.39 nM; R&D Systems), IL-2 (50 ng/mL; R&D Systems), anti-CD3 and anti-CD28 beads (Mouse T activator Dynabeads®, BD) and IGF-1 (R&D Systems; 0, 50, 100 and 200 ng/mL). After 5 days at 37° C., cells were stained with CD25-Pe/Cy7, CD4-APC/Cy7 and FoxP3-APC, and % Tregs of CD4+ cells was measured in all populations of cells treated with different concentrations of IGF-1. Proliferation of Tregs was measured by analyzing the dilution of CFSE label in the FITC channel. After the acquisition, all gates and data analyses were performed using the FlowJo® 8.7 software.

### Example 2

This Example discloses the results of treatment of splenocytes-derived regulatory T cells (Tregs; defined as CD4+, CD25+, FoxP3+ cells by flow cytometry) with insulin-like growth factor ligand (IGF-1) at different concentrations and the effect of treatment with istiratumab (MM-141), alone or in combination, wherein treatment with IGF-1 ligand results in increased % Tregs in vitro and treatment with istiratumab inhibits this effect.

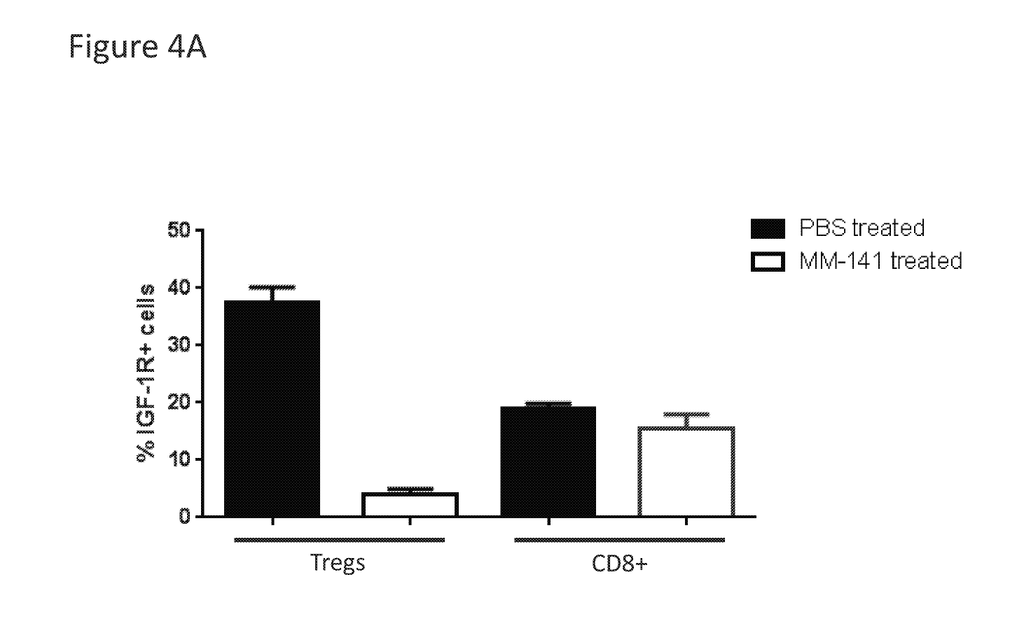

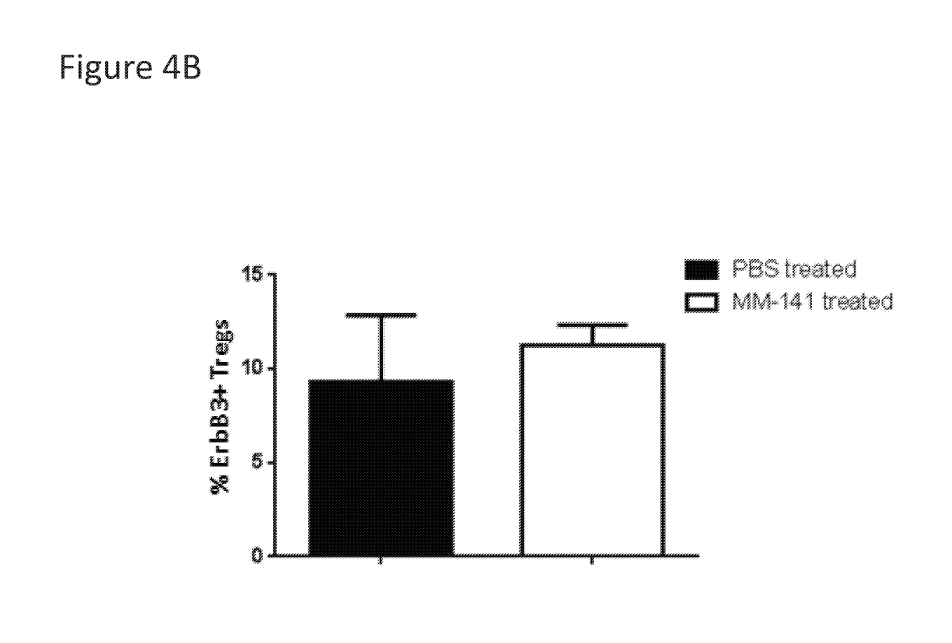

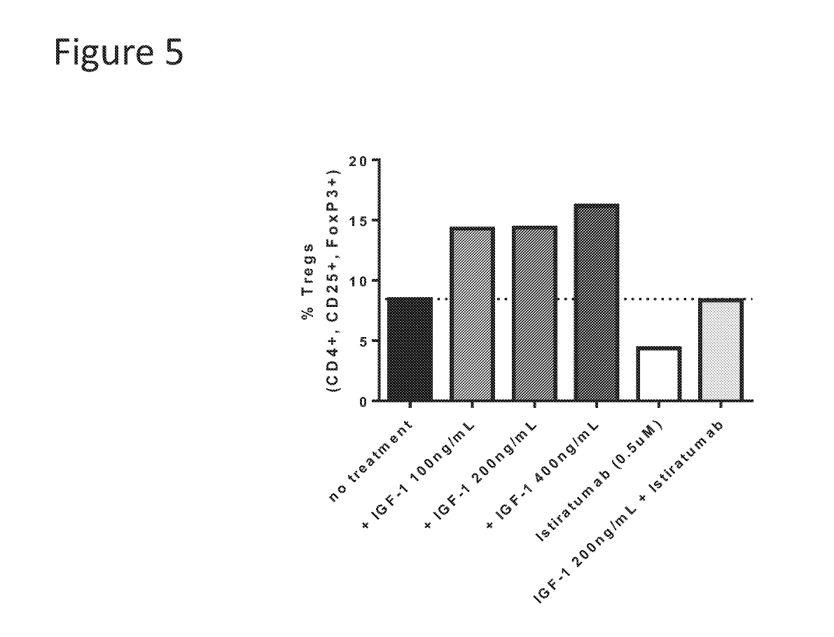

As outlined in Example 1, IGF-1 leads to increased induction of Tregs from CD4+ cells as well as increased proliferation of these Treg cells in vitro. Treatment with istiratumab (MM-141) significantly reduced the expression of IGF-1R on mouse splenocyte derived Tregs but not CD8+ T cells (FIG. 4A). As shown in FIG. 4B, Tregs also express low levels of ErbB3, and these levels are unaffected by treatment with istiratumab. Treatment with istiratumab also decreased % Tregs compared to basal levels (no treatment) and counteracted the increase in % Tregs caused by incubation with IGF-1 (100-400 ng/mL; FIG. 5).

For the experiment relating to FIG. 4, C57BL/6 female mice were inoculated with 10,000 EL4 T lymphoma cells (ATCC® TIB-39) in un-supplemented DMEM medium subcutaneously in the left flank. Mice were divided into two different treatment groups (5 mice/group) and were dosed with PBS (0.2 mL, i.p., q3d) or istiratumab (30 mpk, i.p., q3d) for the duration of the study. Dosing of PBS and istiratumab was initiated on Day 1 post-tumor cell inoculation. Day 26 post-tumor cell inoculation, mice were euthanized and spleens collected, homogenized to single cell suspensions and red blood cells lysed by incubation in ACK buffer (Life Technologies) for 2 minutes. Total cell counts were obtained and aliquots of cells were stained with CD25-Pe/Cy7, CD4-FITC, FoxP3-APC, IGF-1R-biotin and streptavidin-Brilliant Violet™ 421, which is visible in the Pacific Blue channel. All fluorescent antibodies were sourced from BioLegend®. To confirm IGF-1R specific staining, an aliquot of cells was stained without IGF-1R-biotin but containing streptavidin-Brilliant Violet 421 and used to set the gates for calculations. Percent expression of IGF-1R+ Tregs was determined by analyzing the percentage of Tregs (CD4+, CD25+, FoxP3+) expressing IGF-1R as measured by quantitative flow cytometry (FACS Canto, BD Biosciences).

For the quantitative flow cytometry experiments outlined in FIG. 5, spleens were extracted from female wild-type C57BL/6 mice, homogenized to single cell suspension and red blood cells lysed by incubation in ACK buffer (Life Technologies) for 2 minutes. Cells were stained with CD25-PE and CD4-biotin labelled antibodies, and CD4+CD25+ dual marker positive cells were isolated using an AUTOMACS magnetic cell sorter. Isolated CD4+CD25+ cells were plated at 80,000 cells per well in a 96 well plate in media containing IL-2 (50 ng/mL; R&D Systems) and anti-CD3 and anti-CD28 beads (Mouse T activator Dynabeads®, BD). Where applicable, IGF-1 (0, 100, 200 and 400 ng/mL; R&D Systems) and/or istiratumab (0.5 M) were added. After 5 days at 37° C., cells were stained with antibodies conjugated to fluorescent dyes—CD25-Pe/Cy7 (BioLegend), CD4-APC/Cy7 (BioLegend) and FoxP3-APC (Miltenyi Biotec), and % FoxP3+ cells of CD4+CD25+ cells was measured by flow cytometry. After the acquisition, all gates and data analyses were performed using the FlowJo® 8.7 software.

Regulatory T cells (Tregs) comprise a subpopulation of T cells that modulate the immune system response to foreign or aberrant cell types and maintain tolerance to self-antigens. The proliferation of mouse splenocyte-derived Tregs is modulated by several co-stimulatory proteins and is also induced by treatment with IGF-1 ligand. As shown in FIG. 5, treatment with istiratumab decreased the percentage of Tregs below basal levels, as measured by flow cytometry. Furthermore, the effect of IGF-1 on % Tregs is eliminated when cells are co-treated with istiratumab.

### Example 3

This Example describes a method of treatment of cancer (pancreatic cancer, ovarian cancer, hepatocellular carcinoma, parathyroid cancer, sarcoma, B cell lymphoma, lung cancer or breast cancer) with istiratumab. Istiratumab is dosed and administered as described in WIPO Publication No. WO/2015/130554, except that, in accordance with the present disclosure, it is administered as combination therapy with agents not disclosed in WO/2015/130554.

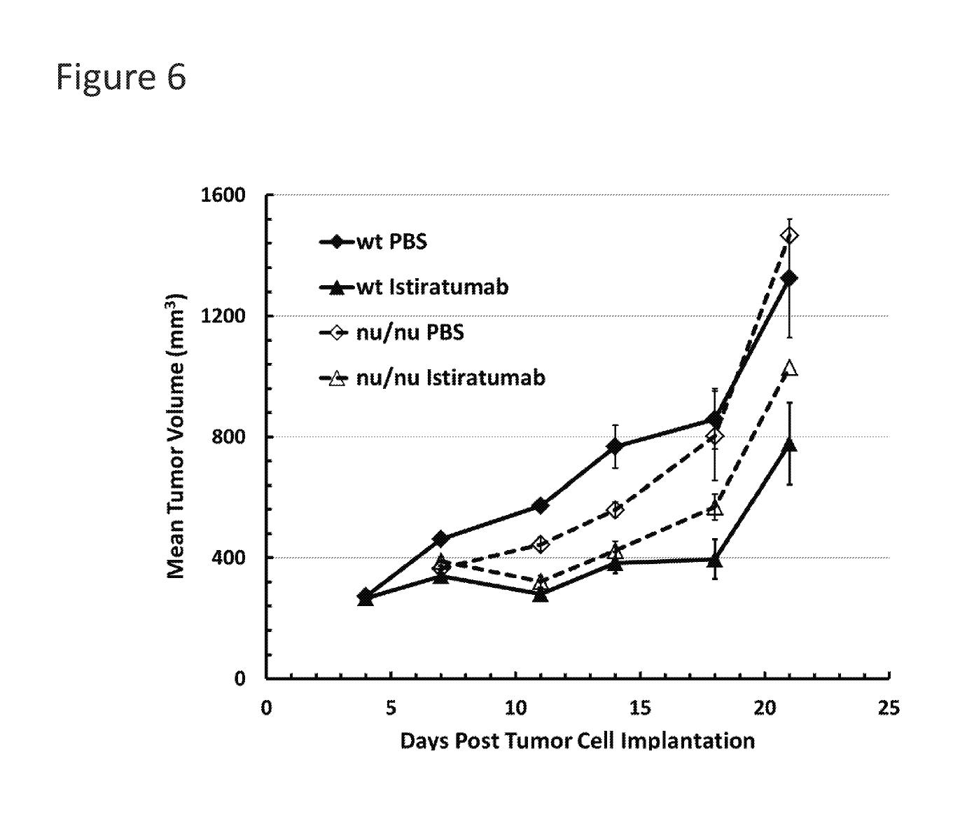

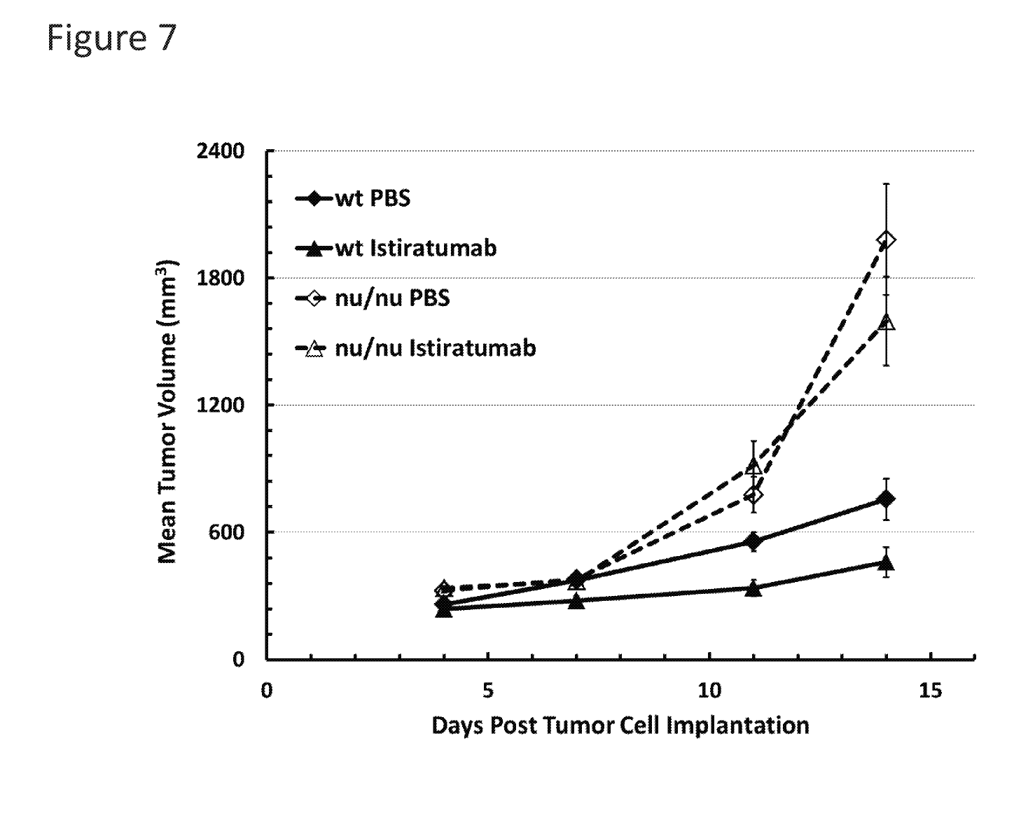

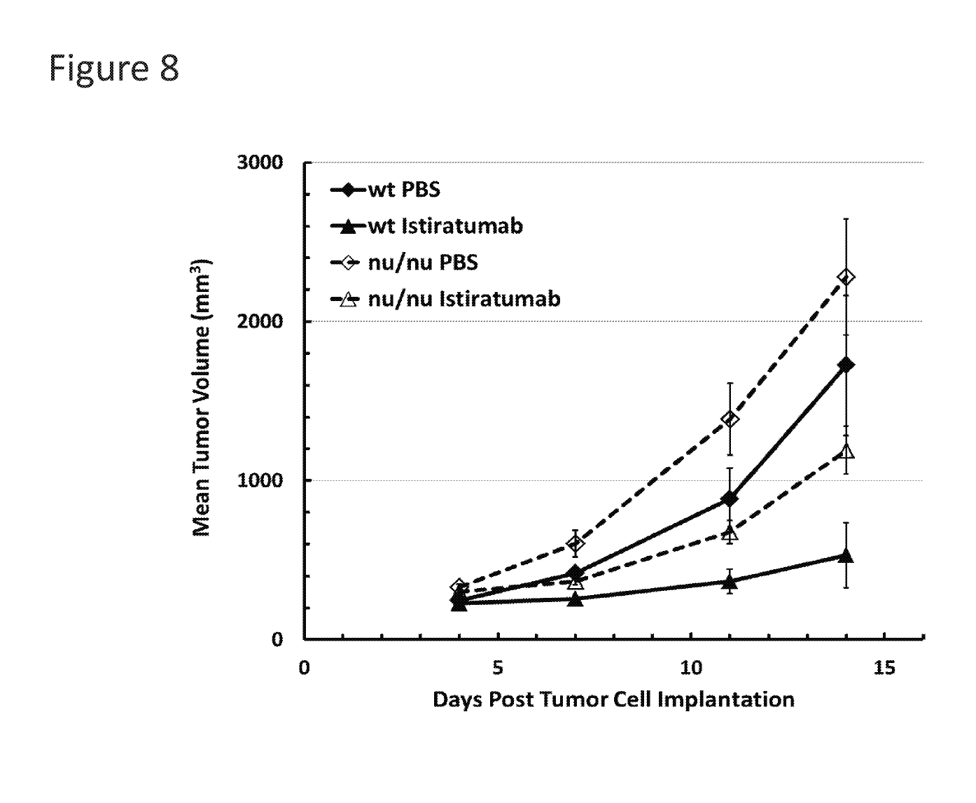

Treating A20 B cell lymphoma tumor-bearing immuno-competent BALB/c or athymic nude mice with istiratumab monotherapy resulted in reduced tumor volume over time compared to treatment with PBS vehicle control (FIG. 6). Istiratumab monotherapy antitumor activity in athymic mice, which are T cell-deficient, suggests that istiratumab-mediated mechanisms may recruit the antitumor activity of innate (non-T cell) immune cells. Istiratumab antitumor activity in immuno-competent mice, which is greater than in athymic mice, suggests istiratumab-mediated mechanisms also recruit the antitumor activity of T cells, in addition to innate immune cells, since all immune cell sub-types are present and functional in the immuno-competent mice. As outlined in FIG. 7, for MC38 tumor-bearing athymic mice, treatment with istiratumab did not result in significant reduction of tumor volume over the course of treatment. In contrast, treatment of MC38 colon tumor-bearing immuno-competent mice with istiratumab monotherapy resulted in a reduction in tumor volume compared to PBS control. These findings suggest that istiratumab-mediated mechanisms may primarily recruit the antitumor activity of T cells in this model. In FIG. 8, B16-F10 melanoma tumor-bearing immuno-competent and athymic mice treated with istiratumab had reduced tumor volume over time in comparison to PBS-treated mice, suggesting there is the potential for multiple, concurrent, istiratumab-mediated antitumor mechanisms in the B16-F10 melanoma model.

As indicated in FIG. 6, female BALB/c (wild-type, immuno-competent; solid symbols and lines) or female athymic nude (nu/nu, T cell-deficient; open symbols, dashed lines) mice (10 mice/group) were inoculated subcutaneously with 6×10⁶ A20 B cell lymphoma cells in un-supplemented RPMI 1640 suspension in the left flank. Beginning on Day 4 post-tumor cell inoculation, the BALB/c mice and athymic nude mice were dosed for the duration of the study with PBS (0.2 mL, i.p., q3d; diamonds) or istiratumab (30 mpk, i.p., q3d; triangles) as described. Tumor volume was measured twice weekly.

For the in vivo study outlined in FIG. 7, female C57BL/6 (wild-type, immuno-competent; solid symbols and lines; 10/group) or female athymic nude (open symbols and dashed lines; 10/group) mice were inoculated subcutaneously with 3×10⁶ MC38 colorectal cells (NCI®) in un-supplemented RPMI 1640 suspension in the left flank. Beginning on Day 4 post-tumor cell inoculation, the C57BL/6 mice and athymic nude mice were dosed for the duration of the study with PBS (0.2 mL. i.p., q3d; diamonds) or istiratumab (30 mpk, i.p., q3d; triangles). Tumor volume was measured twice weekly.

As outlined in FIG. 8, female C57BL/6 (solid symbols and lines; 5/group) or female athymic nude (open symbols and dashed lines; 10/group) mice were inoculated subcutaneously with 5×10⁵ B16-F10 melanoma cells (ATCC® CRL-6475) in un-supplemented DMEM suspension in the left flank. Beginning on Day 4 post-tumor cell inoculation, the mice were dosed for the duration of the study with PBS (0.2 mL. i.p., q3d; diamonds) or istiratumab (30 mpk, i.p., q3d; triangles). Tumor volume was measured twice weekly.

### Example 4

This Example describes a method of treatment of cancer (e.g., B cell lymphoma) with a combination of a checkpoint inhibitor and istiratumab, wherein the therapeutic effect of the combination is larger than the therapeutic effect of an immune checkpoint inhibitor or istiratumab alone when each is administered as monotherapy at the same dose as in the combination. Istiratumab is dosed and administered as specified in Example 3, above, and the immune checkpoint inhibitor is dosed and administered as indicated in FIG. 17.

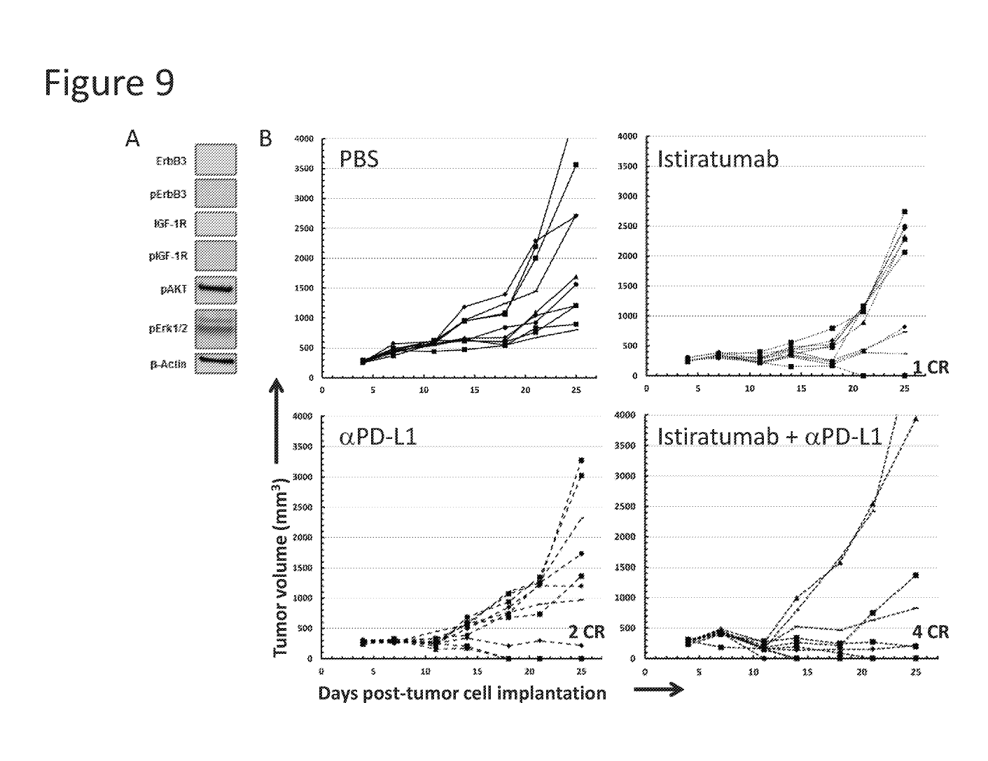

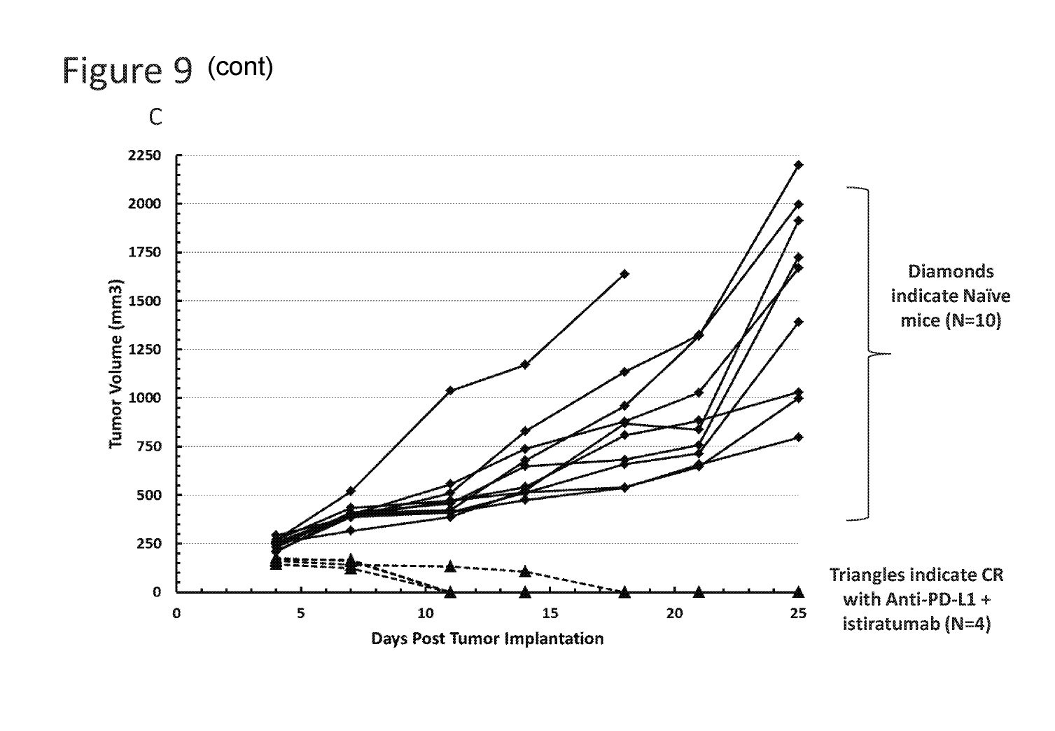

As indicated in FIG. 9A, A20 B cell lymphoma cells (ATCC® TIB-208) did not express IGF-1R or ErbB3 receptors. As shown in FIG. 9B, dosing with istiratumab in combination with anti-PD-L1 antibody was more active at inhibiting the growth of IGF-1R- and ErbB3-non-expressing A20 B cell lymphoma cell line tumors than dosing with either agent alone. As shown in FIG. 9C, mice that had complete responses (CR; no palpable tumors) to treatment with a combination of istiratumab and anti-PD-L1 did not form tumors following re-inoculation with A20 tumor cells two months after stopping treatment with agents.

For the immunoblotting experiments detailed in FIG. 9A, A20 cells were plated on 12 well dishes (3×10⁵ cells per well) in 10% serum-containing medium and incubated overnight. Once the cell density had reached approximately 70%, cells were harvested in lysis buffer containing protease and phosphatase inhibitors and analyzed by western blotting.

For the in vivo study outlined in FIG. 9B, BALB/c female mice were inoculated with 6×10⁶ A20 cells in un-supplemented RPMI 1640 medium subcutaneously in the left flank. Mice were divided into four different treatment groups (10 mice/group) and were dosed with PBS (0.2 mL, i.p., q3d), istiratumab (30 mpk, i.p., q3d), anti-PD-L1 (MPDL3280a/atezolizumab; 200 μg/mouse per dose, i.p., 3 time a week: Monday, Wednesday, Friday), or the combination of istiratumab and anti-PD-L1, each as dosed for the monotherapy, for the duration of the study. Dosing of istiratumab was initiated on Day 1 post-tumor cell inoculation and dosing of anti-PD-L1 was initiated on Day 4 post-tumor cell inoculation. Tumor volume was measured twice weekly. For the re-challenge assay shown in FIG. 9C, the four complete responder (CR) mice treated with anti-PD-L1+istiratumab of FIG. 9B (lower right panel) were re-challenged by inoculation with 10 million A20 cells subcutaneously into the right flank (the flank not previously used). Ten naïve BALB/c female mice were inoculated with 10 million A20 cells at the same time as a comparative control. Tumor volume was measured twice weekly.

### Example 5

This Example describes a method of treatment of cancer (e.g., fibrosarcoma) with a combination of an antibody of FIG. 17 and istiratumab, wherein the therapeutic effect of the combination is larger than the therapeutic effect of an immune checkpoint inhibitor or istiratumab alone when each is administered as monotherapy at the same dose as in the combination.

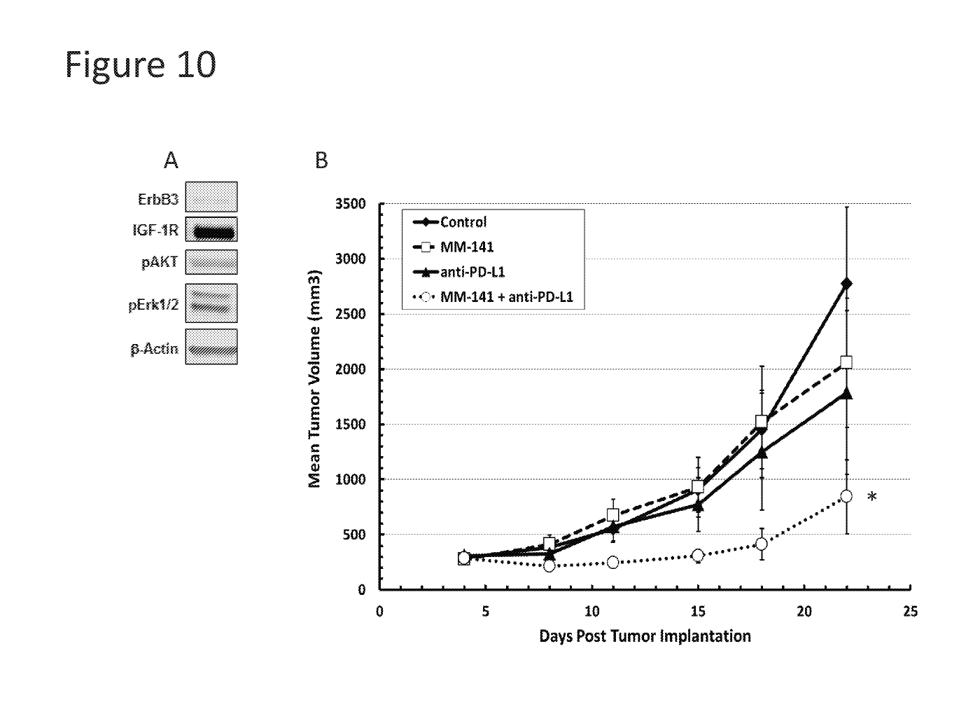

As indicated in FIG. 10A, WEHI164 fibrosarcoma cells (ATCC® CRL-1751) expressed IGF-1R and ErbB3 receptors. As shown in FIG. 10B, dosing with istiratumab in combination with anti-PD-L1 antibody was more active at inhibiting the growth of IGF-1R- and ErbB3-expressing WEHI164 fibrosarcoma cell line tumors than dosing with either agent alone.

For the immunoblotting experiments detailed in FIG. 10A, WEHI164 cells were plated on 12 well dishes (3×10⁵ cells per well) in 10% serum-containing medium and incubated overnight. Once the cell density had reached approximately 70%, cells were harvested in lysis buffer containing protease and phosphatase inhibitors and analyzed by western blotting.

For the in vivo study outlined in FIG. 10B, BALB/c female mice were inoculated with 4.5×10⁶ WEHI164 fibrosarcoma cells in un-supplemented DMEM medium subcutaneously in the left flank. Mice were divided to four different treatment groups (10 mice/group) and were dosed with PBS (0.2 mL, i.p., q3d; black diamonds, solid black line), istiratumab (30 mpk, i.p., q3d; open squares, dashed line), anti-PD-L1 (atezolizumab/MPDL3280a; 200 μg/mouse per dose, i.p., three times weekly (Monday, Wednesday, Friday); solid triangles, solid black line), or the combination of istiratumab and anti-PD-L1 (open circles, dotted line) as dosed for the monotherapy for the duration of the study. Dosing of istiratumab was initiated on Day 1 post-tumor cell inoculation and dosing of anti-PD-L1 was initiated on Day 4 post-tumor cell inoculation. Tumor volume was measured twice weekly.

### Example 6

This Example describes a method of treatment of cancer (e.g., colon carcinoma) with a combination of an antibody of FIG. 17 and istiratumab, wherein the therapeutic effect of the combination is larger than the therapeutic effect of an immune checkpoint inhibitor or istiratumab alone when each is administered as monotherapy at the same dose as in the combination.

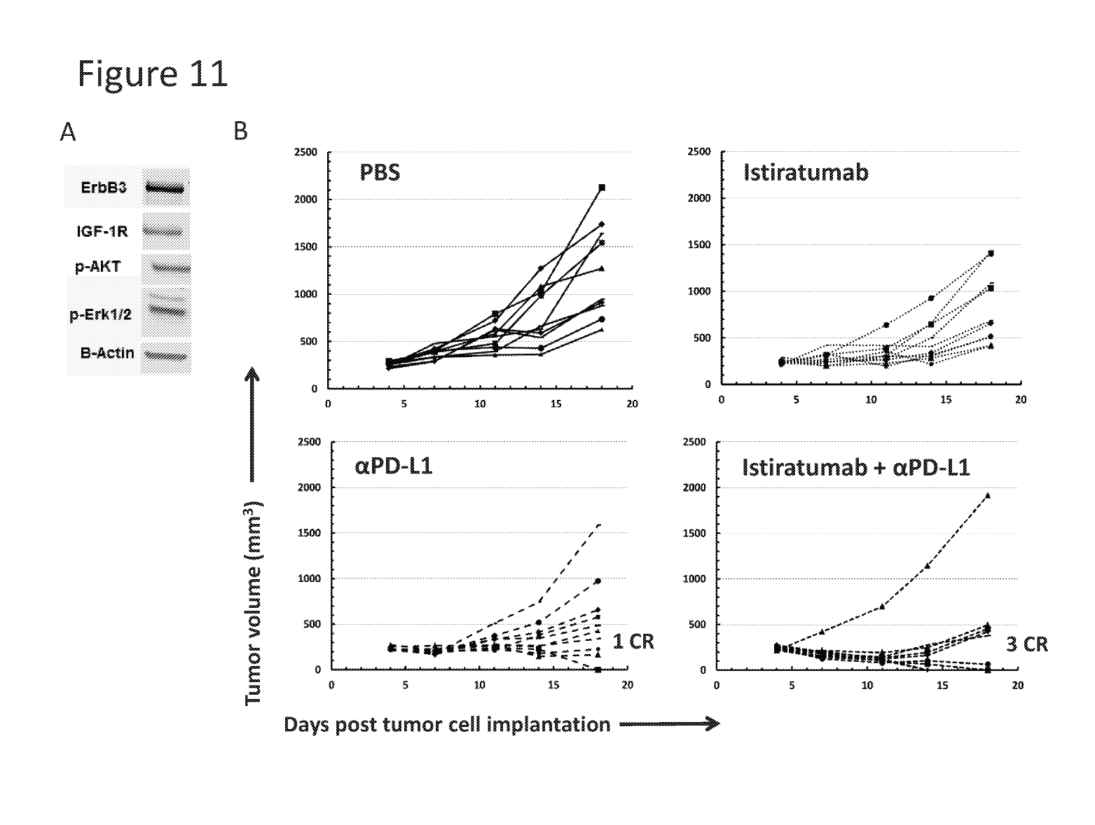

FIG. 11A shows that MC38 colorectal cells express IGF-1R and ErbB3. As shown in FIG. 11B, while most of the mice treated with the checkpoint inhibitor anti-PD-L1 had reduced tumor volume over time, including one complete response, mice treated with both istiratumab and anti-PD-L1 showed an even greater overall response, including 3 complete responses.

For the immunoblotting experiment detailed in FIG. 11A, MC38 cells were plated on 12 well dishes (3×10⁵ cells per well) in 10% serum-containing media and incubated overnight. Once the cell density had reached approximately 70%, cells were harvested in lysis buffer containing protease and phosphatase inhibitors and were analyzed by western blotting.

For the in vivo study outlined in FIG. 11B, female C57BL/6 mice (10/group) were inoculated subcutaneously with 3×10⁶ MC38 colorectal cancer cells in un-supplemented RPMI 1640 suspension in the left flank. Beginning on Day 4 post-tumor cell inoculation, the mice were dosed for the duration of the study with PBS (0.2 mL. i.p., q3d), istiratumab (30 mpk, i.p., q3d), anti-PD-L1 (atezolizumab/MPDL3280a; 100 μg per dose, i.p., three times weekly (Monday, Wednesday, Friday)), or the combination of istiratumab and anti-PD-L1 as dosed for the monotherapy. Tumor volume was measured twice weekly.

### Example 7

This Example describes a method of treatment of cancer (e.g., melanoma) with a combination of an antibody of FIG. 17 and istiratumab, wherein the therapeutic effect of the combination is larger than the therapeutic effect of an immune checkpoint inhibitor or istiratumab alone when each is administered as monotherapy at the same dose as in the combination.

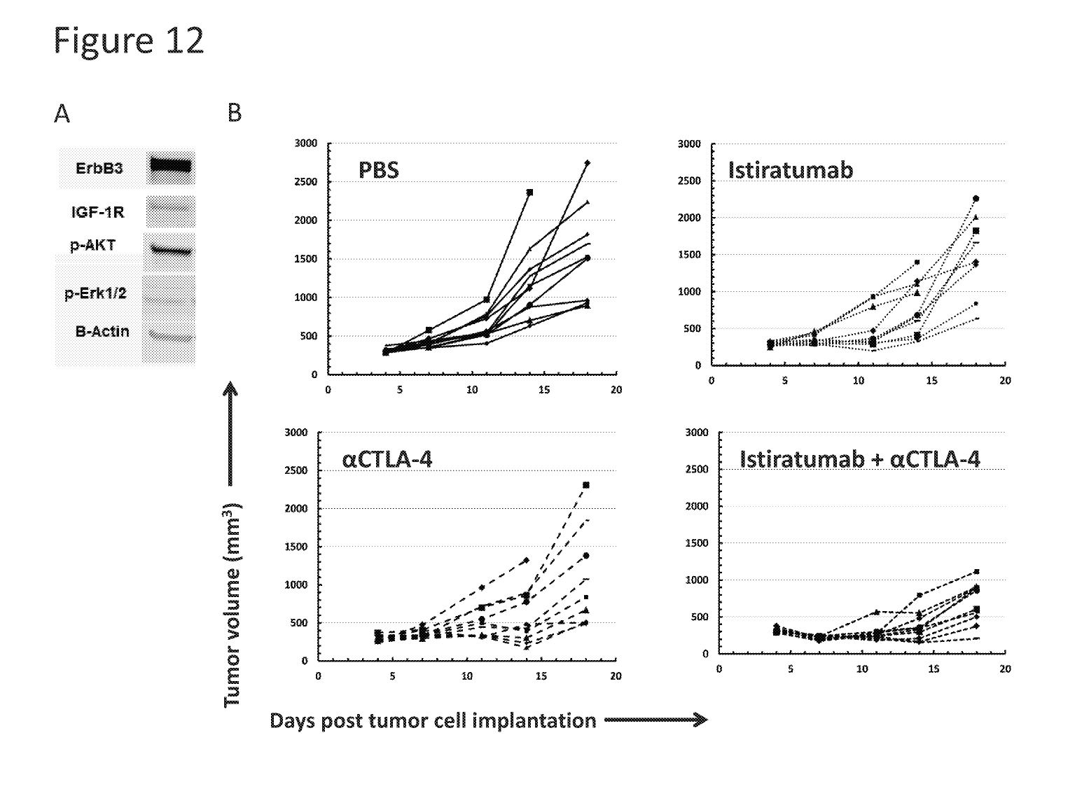

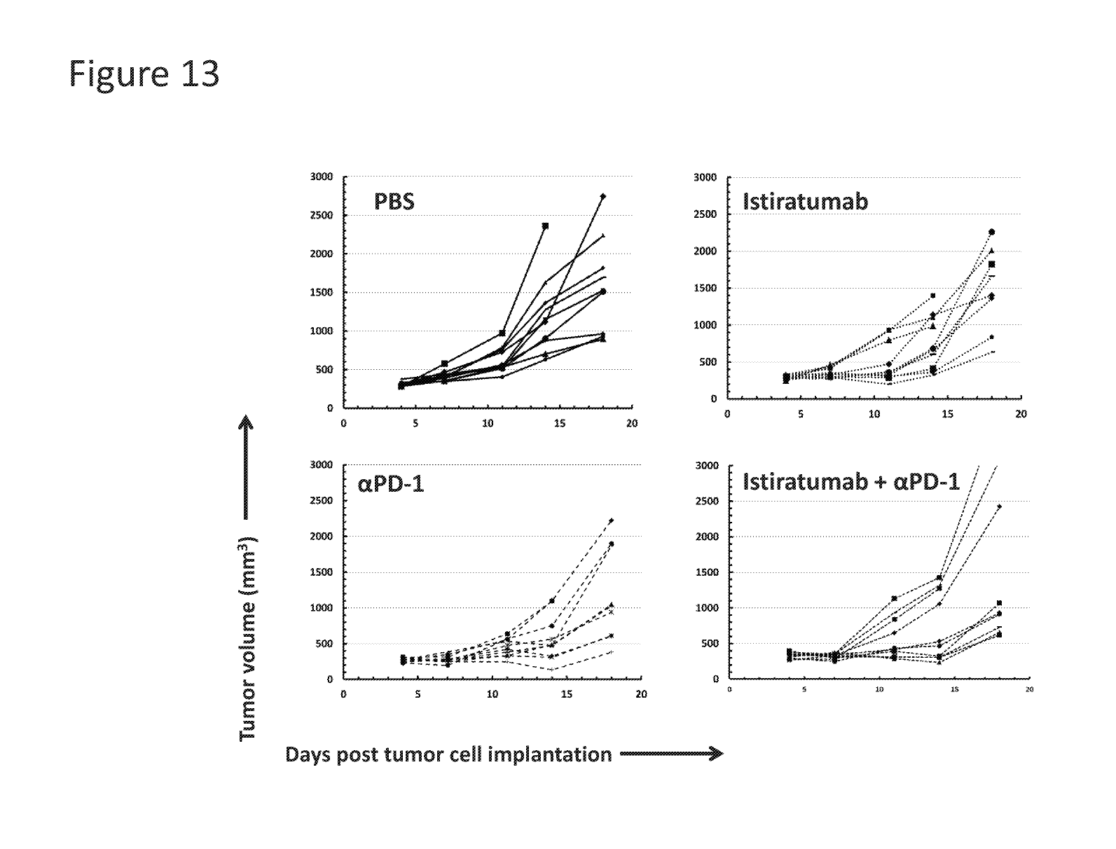

B16-F10 melanoma cells express IGF-1R and ErbB3 (FIG. 12A). As shown in FIG. 12B, while a subset of the mice treated with the checkpoint inhibitor anti-CTLA-4 had reduced tumor volume over time, mice treated with both istiratumab and anti-CTLA-4 showed an even greater overall response. In addition, mice treated with istiratumab and an anti-PD-1 inhibitor had an improved anti-tumor response (FIG. 13).

For the immunoblotting experiment detailed in FIG. 12A, B16-F10 cells were plated on 12 well dishes (3×10⁵ cells per well) in 10% serum-containing media and incubated overnight. Once the cell density had reached approximately 70%, cells were harvested in lysis buffer containing protease and phosphatase inhibitors and were analyzed by western blotting.

For the in vivo study outlined in FIG. 12B, female C57BL/6 mice (10/group) were inoculated subcutaneously with 5×10⁵ B16-F10 melanoma cells in un-supplemented DMEM suspension in the left flank. Beginning on Day 4 post-tumor cell inoculation, the mice were dosed with PBS (0.2 mL. i.p., q3d), istiratumab (30 mpk, i.p., q3d), anti-CTLA-4 (clone 9D9, a mouse IgG2b monoclonal antibody, Catalog #: BE0164, BioXCell®; 100 μg per dose, i.p., q3d, on Days 4, 7 and 10 only), or the combination of istiratumab and anti-CTLA-4 as dosed for the monotherapy. Mice were dosed with PBS or istiratumab for the duration of the study. Tumor volume was measured twice weekly.

For the in vivo study outlined in FIG. 13, female C57BL/6 mice (10/group) were inoculated subcutaneously with 5×10⁵ B16-F10 melanoma cells in un-supplemented DMEM suspension in the left flank. Beginning on Day 4 post-tumor cell inoculation, the mice were dosed with PBS (0.2 mL. i.p., q3d), istiratumab (30 mpk, i.p., q3d), anti-PD-1 (in-house generated mouse IgG2a version of clone J43 hamster antibody; 100 μg per dose, i.p., Days 4, 8, 11, 14 and 18 only), or the combination of istiratumab and anti-PD-1 as dosed for the monotherapy. Mice were dosed with PBS or istiratumab for the duration of the study. Tumor volume was measured twice weekly.

### Example 8

This Example discloses the results of treatment of plasmacytoid dendritic cells (pDCs; defined as CD123+CD303+) with the ligands insulin-like growth factor ligand (IGF-1) and heregulin (HRG), and istiratumab, wherein the treatment of pDCs with IGF-1 and HRG inhibits the activation of pDCs (as measured by expression of the cell surface activation markers CD83 and CD86 by flow cytometry, and interferon alpha (IFNα) production in the culture medium by ELISA) and PD-L1 expression (measured by flow cytometry) and treatment with istiratumab does not affect pDC activity. Plasmacytoid dendritic cells derived from human whole blood express high levels of IGF-1R and ErbB3, as determined by flow cytometry.

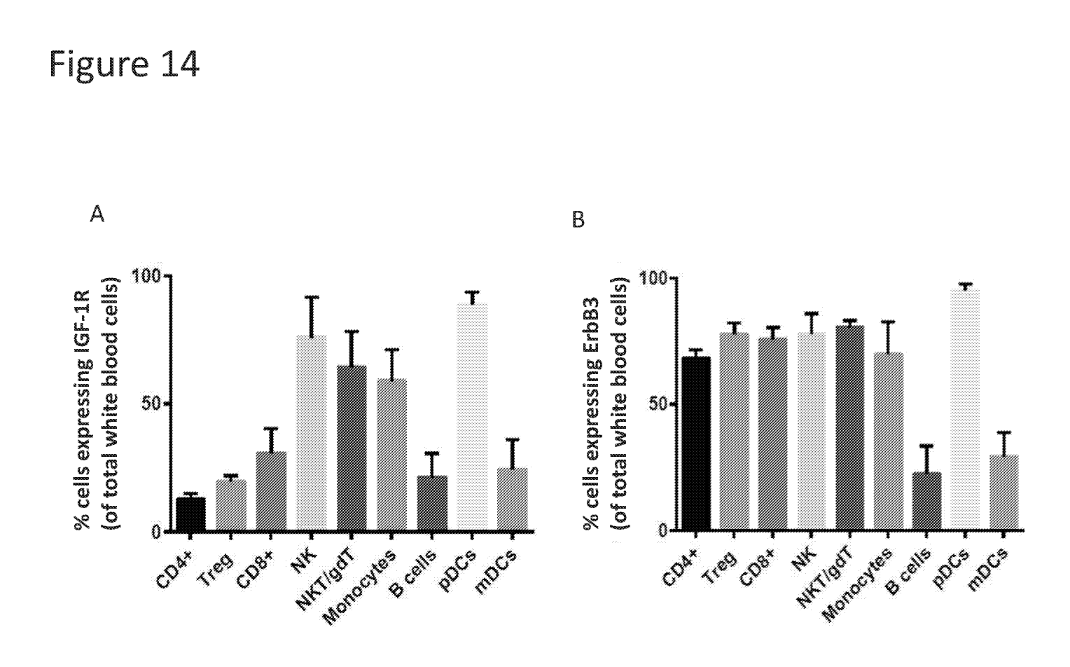

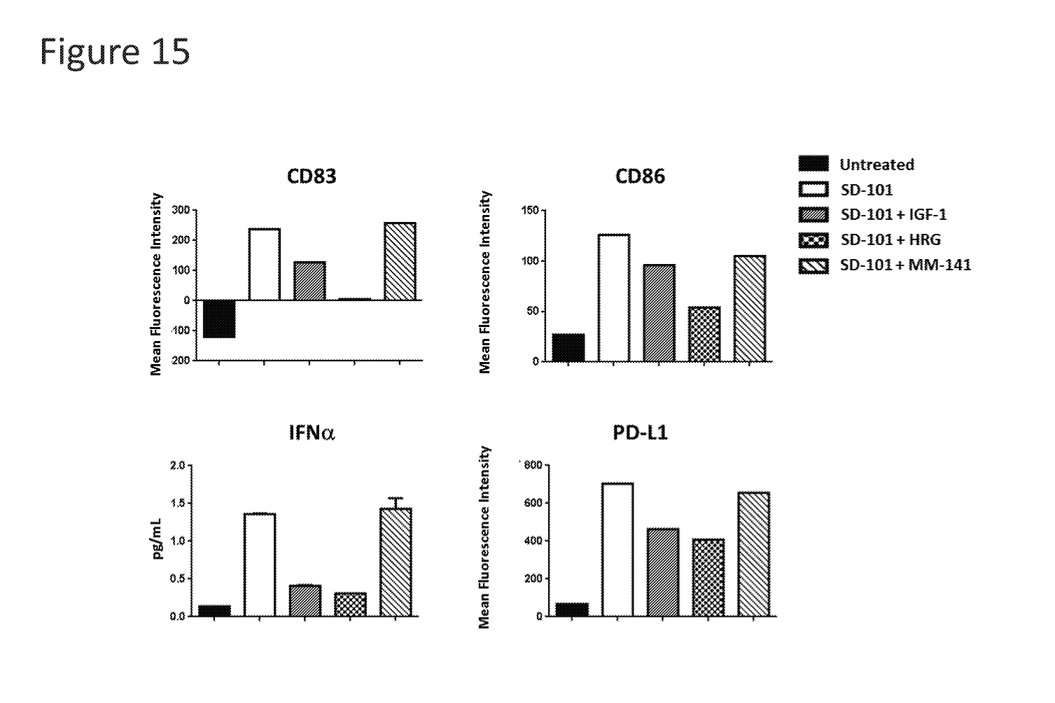

Plasmacytoid dendritic cells are a subpopulation of innate immune cells that circulate in the blood and are found in peripheral lymphoid organs. They constitute less than 0.4% of peripheral blood mononuclear cells. In humans, these cells express the surface markers CD123 and CD303. Upon stimulation and subsequent activation, these cells produce large amounts of type I interferon (mainly IFN-α (alpha) and IFN-3 (beta)), which are critical pleiotropic anti-viral compounds mediating a wide range of effects. Based on flow cytometry analyses, pDCs express high levels of cell surface IGF-1R and ErbB3 receptors (FIG. 14). Inhibition of pDC activity by ligand treatment (FIG. 15) suggests that blocking these ligands will increase the activity of pDCs and thereby potentially improve the anti-tumor immune response.

For the experiments outlined in FIG. 14, human whole blood was collected from 3 disease free patients in non-EDTA containing K2 Vacutainers®. The white blood cell fraction was isolated from the whole blood by centrifugation over Ficoll-Paque™ (GE Healthcare: Life Sciences). Various cell immune cell populations were defined by cell surface marker expression as follows (CD4+ T cells, CD4+; Tregs, CD4+CD25+GITR+; CD8+ T cells, CD8+; NK, CD56+CD3-; NKT, CD56+CD3+; monocytes, CD14+; B cells, CD19+; pDCs, CD123+CD303+; mDCs, CD19-CD1c+) and tested for co-expression of IGF-1R and ErbB3 receptor using specific antibodies, as measured by flow cytometry.

For the quantitative flow cytometry experiments outlined in FIG. 15, human whole blood was collected from disease free patients in non-EDTA containing K2 Vacutainers®. The white blood cell fraction was isolated from the whole blood by centrifugation over Ficoll-Paque™ (GE Healthcare: Life Sciences) and red blood cells lysed by incubation in ACK buffer (Life Technologies) for 2 minutes. Total cell counts were obtained and pDCs were isolated by negative enrichment using the human pDC isolation kit (Miltenyi Biotec) and an AUTOMACS magnetic cell sorter. Isolated pDCs (defined as CD123+CD303+) were >80% pure, based on CD123+CD303+ purity analysis. CD123+CD303+ cells were plated at 20,000 cells per well in a 96 well plate in RPMI media containing 10% fetal bovine serum and betamercaptoethanol (50 μM) in the presence or absence of SD-101 (a Toll-like receptor 9 agonist; 0.5 μM), IGF-1 (50 nM), HRG (10 nM) or istiratumab (300 nM) for 16 hours at 37 degrees Celsius. Changes in percent expression of CD83 and CD86 (markers of pDC activity, represented as mean fluorescence intensity) and interferon α expression in the cell culture media were quantified using flow cytometry and ELISA, respectively.

### Example 9

This Example discloses the results of treatment of myeloid-derived suppressor cells (MDSCs; defined as Gr1^{dim} Ly6C negative (Ly6C⁻)) with istiratumab, wherein the treatment of MDSCs with istiratumab inhibited the suppressive activity of this cell population on CD4+ T cell proliferation.

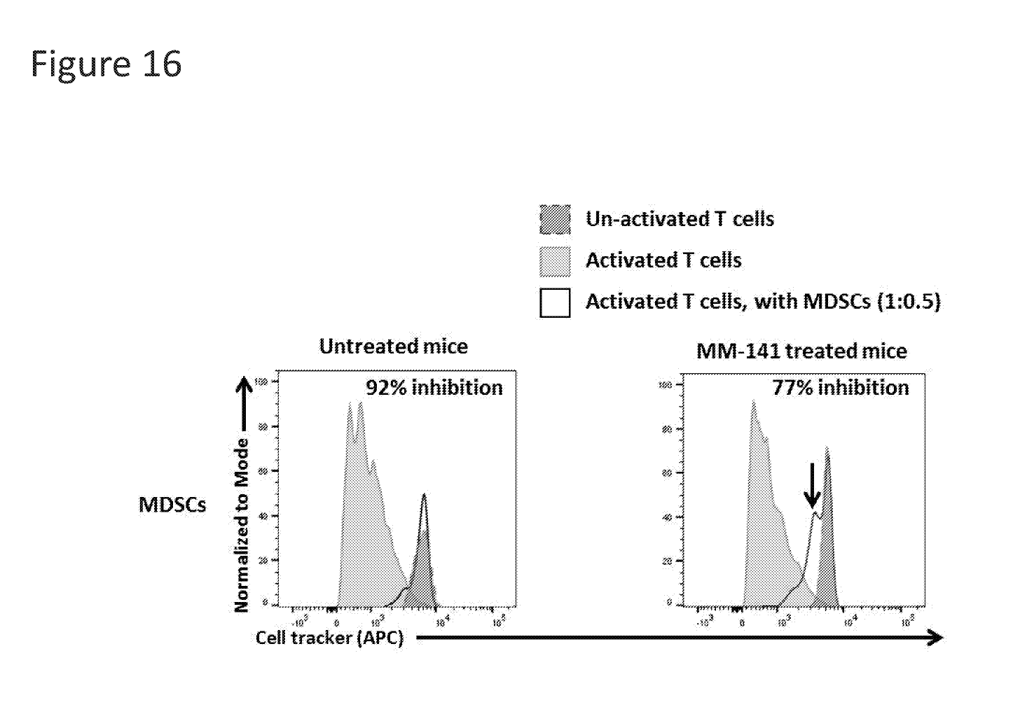

Similar to Tregs, MDSCs are suppressors of immune activation, which can dampen the proliferation of immune cells, including CD4+ T cells. Treatment of mice with istiratumab in vivo dampens the ability of MDSCs isolated from the spleens of these mice to suppress activated CD4+ T cell proliferation in vitro, with 92% inhibition of CD4+ T cell proliferation in the presence of MDSCs from untreated mice compared to 77% inhibition of CD4+ T cell proliferation in the presence of MDSCs from istiratumab-treated mice (FIG. 16).

As outlined in FIG. 16, two C57bl/6 mice were treated with istiratumab (30 mpk, i.p. q3d×3). Spleens were isolated from istiratumab-treated mice for MDSC isolation and two untreated C57bl/6 mice for CD4+ T cell isolation. MDSCs and CD4+ T cells were isolated using the MDSC isolation kit (Miltenyi Biotec) and CD4+ T cell isolation kit (Miltenyi Biotec), respectively, and an AUTOMACS magnetic cell sorter. MDSCs were stained with CFSE and CD4+ T cells were stained with Far Red Cell tracker (Thermo Fisher), then 50,000 CD4+ T cells were plated together with 25,000 MDSCs, where applicable, at 1:0, 1:0.5 and 0:1 CD4+:MDSCs for 3 days in the presence or absence of CD3/CD28 beads (activators of CD4+ T cells). CD4+ T cell proliferation was measured by evaluating Far Red Cell Tracker dilution in the APC channel by flow cytometry. Unactivated CD4+ T cells did not proliferate (as evidenced by the single right-most peak), whereas activated CD4+ T cells did proliferate, as evidenced by the leftward shift (i.e. decrease) in Far Red Cell Tracker signal.

### ENDNOTES

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features set forth herein. The disclosure of each and every U.S., international, or other patent or patent application or publication referred to herein is hereby incorporated herein by reference in its entirety.

ADDITIONAL SEQUENCES

| SEQ ID | | |
|---|---|---|
| NO: | DESIGNATION | SEQUENCE |
| 438 | MPDL3280A_HC_ | EVQLVESGGGLVQPGGSLRLSCAASGFTFSDSWIHWVRQAPGKGL |
| | hIgG1_ | EWVAWISPYGGSTYYADSVKGRFTISADTSKNTAYLQMNSLRAED |
| | D265A/N297A | TAVYYCARRHWPGGFDYWGQGTLVTVSAASTKGPSVFPLAPSSKS |
| | | TSGGTAALGCLVKDYFPEPVTVSWNSGALTSGVHTFPAVLQSSGL |
| | | YSLSSVVTVPSSSLGTQTYICNVNHKPSNTKVDKKVEPKSCDKTH |
| | | TCPPCPAPELLGGPSVFLFPPKPKDTLMISRTPEVTCVVVAVSHE |
| | | DPEVKFNWYVDGVEVHNAKTKPREEQYASTYRVVSVLTVLHQDWL |
| | | NGKEYKCKVSNKALPAPIEKTISKAKGQPREPQVYTLPPSRDELT |
| | | KNQVSLTCLVKGFYPSDIAVEWESNGQPENNYKTTPPVLDSDGSF |
| | | FLYSKLTVDKSRWQQGNVFSCSVMHEALHNHYTQKSLSLSPG |
| 439 | MPDL3280A_HC_ | EVQLVESGGGLVQPGGSLRLSCAASGFTFSDSWIHWVRQAPGKGL |
| | chimerized_ | EWVAWISPYGGSTYYADSVKGRFTISADTSKNTAYLQMNSLRAED |
| | mIgG2A_ | TAVYYCARRHWPGGFDYWGQGTLVTVSAAKTTAPSVYPLAPVCGD |
| | D265A/N297A | TTGSSVTLGCLVKGYFPEPVTLTWNSGSLSSGVHTFPAVLQSDLY |
| | | TLSSSVTVTSSTWPSQSITCNVAHPASSTKVDKKIEPRGPTIKPC |
| | | PPCKCPAPNLLGGPSVFIFPPKIKDVLMISLSPIVTCVVVAVSED |
| | | DPDVQISWFVNNVEVHTAQTQTHREDYASTLRVVSALPIQHQDWM |
| | | SGKEFKCKVNNKDLPAPIERTISKPKGSVRAPQVYVLPPPEEEMT |
| | | KKQVTLTCMVTDFMPEDIYVEWTNNGKTELNYKNTEPVLDSDGSY |
| | | FMYSKLRVEKKNWVERNSYSCSVVHEGLHNHHTTKSFSRTPG |
| 440 | MPDL3280A_LC | DIQMTQSPSSLSASVGDRVTITCRASQDVSTAVAWYQQKPGKAPK |
| | | LLIYSASFLYSGVPSRFSGSGSGTDFTLTISSLQPEDFATYYCQQ |
| | | YLYHPATFGQGTKVEIKRTVAAPSVFIFPPSDEQLKSGTASVVCL |
| | | LNNFYPREAKVQWKVDNALQSGNSQESVTEQDSKDSTYSLSSTLT |
| | | LSKADYEKHKVYACEVTHQGLSSPVTKSFNRGEC |
| 441 | MPDL3280A_LC_ | DIQMTQSPSSLSASVGDRVTITCRASQDVSTAVAWYQQKPGKAPK |
| | chv2 | LLIYSASFLYSGVPSRFSGSGSGTDFTLTISSLQPEDFATYYCQQ |
| | | YLYHPATFGAGTKLELKRADAAPTVSIFPPSSEQLTSGGASVVCF |
| | | LNNFYPKDINVKWKIDGSERQNGVLNSWTDQDSKDSTYSMSSTLT |
| | | LTKDEYERHNSYTCEATHKTSTSPIVKSFNRNEC |
| 442 | 141mIgG2aHC | EVQLLQSGGGLVQPGGSLRLSCAASGFMFSRYPMHWVRQAPGKGL |
| | | EWVGSISGSGGATPYADSVKGRFTISRDNSKNTLYLQMNSLRAED |
| | | TAVYYCAKDFYQILTGNAFDYWGQGTTVTVSSAKTTAPSVYPLAP |
| | | VCGDTTGSSVTLGCLVKGYFPEPVTLTWNSGSLSSGVHTFPAVLQ |
| | | SDLYTLSSSVTVTSSTWPSQSITCNVAHPASSTKVDKKIEPRGPT |
| | | IKPCPPCKCPAPNLLGGPSVFIFPPKIKDVLMISLSPIVTCVVVD |
| | | VSEDDPDVQISWFVNNVEVHTAQTQTHREDYNSTLRVVSALPIQH |
| | | QDWMSGKEFKCKVNNKDLPAPIERTISKPKGSVRAPQVYVLPPPE |
| | | EEMTKKQVTLTCMVTDFMPEDIYVEWTNNGKTELNYKNTEPVLDS |
| | | DGSYFMYSKLRVEKKNWVERNSYSCSVVHEGLHNHHTTKSFSRTP |
| | | GGGGGSGGGGSGGGGSQVQLVQSGGGLVQPGGSLRLSCAASGFTF |
| | | DDYAMHWVRQAPGKGLEWVAGISWDSGSTGYADSVKGRFTISRDN |
| | | AKNSLYLQMNSLRAEDTALYYCARDLGAYQWVEGFDYWGQGTLVT |
| | | VSSASTGGGGSGGGGSGGGGSGGGGSSYELTQDPAVSVALGQTVR |
| | | ITCQGDSLRSYYASWYQQKPGQAPVLVIYGKNNRPSGIPDRFSGS |
| | | TSGNSASLTITGAQAEDEADYYCNSRDSPGNQWVFGGGTKVTVLG |
| 443 | 141mLC | DIQMTQSPSSLSASLGDRVTITCRASQGISSYLAWYQQKPGKAPK |
| | | LLIYAKSTLQSGVPSRFSGSGSGTDFTLTISSLQPEDSATYYCQQ |
| | | YWTFPLTFGGGTKVEIKRADAAPTVSIFPPSSEQLTSGGASVVCF |
| | | LNNFYPKDINVKWKIDGSERQNGVLNSWTDQDSKDSTYSMSSTLT |
| | | LTKDEYERHNSYTCEATHKTSTSPIVKSFNRNEC |
| 444 | ErbB3 protein | Ser Glu Val Gly Asn Ser Gln Ala Val Cys |
| | (corresponding to | Pro Gly Thr Leu Asn Gly Leu Ser Val Thr |
| | SEQ ID NO: 4 | Gly Asp Ala Glu Asn Gln Tyr Gln Thr Leu |
| | from U.S. Pat. | Tyr Lys Leu Tyr Glu Arg Cys Glu Val Val |
| | No. 5,480,968) | Met Gly Asn Leu Glu Ile Val Leu Thr Gly |
| | | His Asn Ala Asp Leu Ser Phe Leu Gln Trp |
| | | Ile Arg Glu Val Thr Gly Tyr Val Leu Val |
| | | Ala Met Asn Glu Phe Ser Thr Leu Pro Leu |
| | | Pro Asn Leu Arg Val Val Arg Gly Thr Gln |
| | | Val Tyr Asp Gly Lys Phe Ala Ile Phe Val |
| | | Met Leu Asn Tyr Asn Thr Asn Ser Ser His |
| | | Ala Leu Arg Gln Leu Arg Leu Thr Gln Leu |
| | | Thr Glu Ile Leu Ser Gly Gly Val Tyr Ile |
| | | Glu Lys Asn Asp Lys Leu Cys His Met Asp |
| | | Thr Ile Asp Trp Arg Asp Ile Val Arg Asp |
| | | Arg Asp Ala Glu Ile Val Val Lys Asp Asn |
| | | Gly Arg Ser Cys Pro Pro Cys His Glu Val |
| | | Cys Lys Gly Arg Cys Trp Gly Pro Gly Ser |
| | | Glu Asp Cys Gln Thr Leu Thr Lys Thr Ile |
| | | Cys Ala Pro Gln Cys Asn Gly His Cys Phe |
| | | Gly Pro Asn Pro Asn Gln Cys Cys His Asp |
| | | Glu Cys Ala Gly Gly Cys Ser Gly Pro Gln |
| | | Asp Thr Asp Cys Phe Ala Cys Arg His Phe |
| | | Asn Asp Ser Gly Ala Cys Val Pro Arg Cys |
| | | Pro Gln Pro Leu Val Tyr Asn Lys Leu Thr |
| | | Phe Gln Leu Glu Pro Asn Pro His Thr Lys |
| | | Tyr Gln Tyr Gly Gly Val Cys Val Ala Ser |
| | | Cys Pro His Asn Phe Val Val Asp Gln Thr |
| | | Ser Cys Val Arg Ala Cys Pro Pro Asp Lys |
| | | Met Glu Val Asp Lys Asn Gly Leu Lys Met |
| | | Cys Glu Pro Cys Gly Gly Leu Cys Pro Lys |
| | | Ala Cys Glu Gly Thr Gly Ser Gly Ser Arg |
| | | Phe Gln Thr Val Asp Ser Ser Asn Ile Asp |
| | | Gly Phe Val Asn Cys Thr Lys Ile Leu Gly |
| | | Asn Leu Asp Phe Leu Ile Thr Gln Gly Asp |
| | | Pro Trp His Lys Ile Pro Ala Leu Asp Pro |
| | | Glu Lys Leu Asn Val Phe Arg Thr Val Arg |
| | | Glu Ile Thr Gly Tyr Leu Asn Ile Gln Ser |
| | | Trp Pro Pro His Met His Asn Phe Ser Val |
| | | Phe Ser Asn Leu Thr Thr Ile Gly Gly Arg |
| | | Ser Leu Tyr Asn Arg Gly Phe Ser Leu Leu |
| | | Ile Met Lys Asn Leu Asn Val Thr Ser Leu |
| | | Gly Phe Arg Ser Leu Lys Glu Ile Ser Ala |
| | | Gly Arg Ile Tyr Ile Ser Ala Asn Arg Gln |
| | | Leu Cys Tyr His His Ser Leu Asn Trp Thr |
| | | Lys Val Leu Arg Gly Pro Thr Glu Glu Arg |
| | | Leu Asp Ile Lys His Asn Arg Pro Arg Arg |
| | | Asp Cys Val Ala Glu Gly Lys Val Cys Asp |
| | | Pro Leu Cys Ser Ser Gly Gly Cys Trp Gly |
| | | Pro Gly Pro Gly Gln Cys Leu Ser Cys Arg |
| | | Asn Tyr Ser Arg Gly Gly Val Cys Val Thr |
| | | His Cys Asn Phe Leu Asn Gly Glu Pro Arg |
| | | Glu Phe Ala His Glu Ala Glu Cys Phe Ser |
| | | Cys His Pro Glu Cys Gln Pro Met Glu Gly |
| | | Thr Ala Thr Cys Asn Gly Ser Gly Ser Asp |
| | | Thr Cys Ala Gln Cys Ala His Phe Arg Asp |
| | | Gly Pro His Cys Val Ser Ser Cys Pro His |
| | | Gly Val Leu Gly Ala Lys Gly Pro Ile Tyr |
| | | Lys Tyr Pro Asp Val Gln Asn Glu Cys Arg |
| | | Pro Cys His Glu Asn Cys Thr Gln Gly Cys |
| | | Lys Gly Pro Glu Leu Gln Asp Cys Leu Gly |
| | | Gln Thr Leu Val Leu Ile Gly Lys Thr His |
| | | Leu Thr Met Ala Leu Thr Val Ile Ala Gly |
| | | Leu Val Val Ile Phe Met Met Leu Gly Gly |
| | | Thr Phe Leu Tyr Trp Arg Gly Arg Arg Ile |
| | | Gln Asn Lys Arg Ala Met Arg Arg Tyr Leu |
| | | Glu Arg Gly Glu Ser Ile Glu Pro Leu Asp |
| | | Pro Ser Glu Lys Ala Asn Lys Val Leu Ala |
| | | Arg Ile Phe Lys Glu Thr Glu Leu Arg Ser |
| | | Leu Lys Val Leu Gly Ser Gly Val Phe Gly |
| | | Thr Val His Lys Gly Val Trp Ile Pro Glu |
| | | Gly Glu Ser Ile Lys Ile Pro Val Cys Ile |
| | | Lys Val Ile Glu Asp Lys Ser Gly Arg Gln |
| | | Ser Phe Gln Ala Val Thr Asp His Met Leu |
| | | Ala Ile Gly Ser Leu Asp His Ala His Ile |
| | | Val Arg Leu Leu Gly Leu Cys Pro Gly Ser |
| | | Ser Leu Gln Leu Val Thr Gln Tyr Leu Pro |
| | | Leu Gly Ser Leu Leu Asp His Val Arg Gln |
| | | His Arg Gly Ala Leu Gly Pro Gln Leu Leu |
| | | Leu Asn Trp Gly Val Gln Ile Ala Lys Gly |
| | | Met Tyr Tyr Leu Glu Glu His Gly Met Val |
| | | His Arg Asn Leu Ala Ala Arg Asn Val Leu |
| | | Leu Lys Ser Pro Ser Gln Val Gln Val Ala |
| | | Asp Phe Gly Val Ala Asp Leu Leu Pro Pro |
| | | Asp Asp Lys Gln Leu Leu Tyr Ser Glu Ala |
| | | Lys Thr Pro Ile Lys Trp Met Ala Leu Glu |
| | | Ser Ile His Phe Gly Lys Tyr Thr His Gln |
| | | Ser Asp Val Trp Ser Tyr Gly Val Thr Val |
| | | Trp Glu Leu Met Thr Phe Gly Ala Glu Pro |
| | | Tyr Ala Gly Leu Arg Leu Ala Glu Val Pro |
| | | Asp Leu Leu Glu Lys Gly Glu Arg Leu Ala |
| | | Gln Pro Gln Ile Cys Thr Ile Asp Val Tyr |
| | | Met Val Met Val Lys Cys Trp Met Ile Asp |
| | | Glu Asn Ile Arg Pro Thr Phe Lys Glu Leu |
| | | Ala Asn Glu Phe Thr Arg Met Ala Arg Asp |
| | | Pro Pro Arg Tyr Leu Val Ile Lys Arg Glu |
| | | Ser Gly Pro Gly Ile Ala Pro Gly Pro Glu |
| | | Pro His Gly Leu Thr Asn Lys Lys Leu Glu |
| | | Glu Val Glu Leu Glu Pro Glu Leu Asp Leu |
| | | Asp Leu Asp Leu Glu Ala Glu Glu Asp Asn |
| | | Leu Ala Thr Thr Thr Leu Gly Ser Ala Leu |
| | | Ser Leu Pro Val Gly Thr Leu Asn Arg Pro |
| | | Arg Gly Ser Gln Ser Leu Leu Ser Pro Ser |
| | | Ser Gly Tyr Met Pro Met Asn Gln Gly Asn |
| | | Leu Gly Glu Ser Cys Gln Glu Ser Ala Val |
| | | Ser Gly Ser Ser Glu Arg Cys Pro Arg Pro |
| | | Val Ser Leu His Pro Met Pro Arg Gly Cys |
| | | Leu Ala Ser Glu Ser Ser Glu Gly His Val |
| | | Thr Gly Ser Glu Ala Glu Leu Gln Glu Lys |
| | | Val Ser Met Cys Arg Ser Arg Ser Arg Ser |
| | | Arg Ser Pro Arg Pro Arg Gly Asp Ser Ala |
| | | Tyr His Ser Gln Arg His Ser Leu Leu Thr |
| | | Pro Val Thr Pro Leu Ser Pro Pro Gly Leu |
| | | Glu Glu Glu Asp Val Asn Gly Tyr Val Met |
| | | Pro Asp Thr His Leu Lys Gly Thr Pro Ser |
| | | Ser Arg Glu Gly Thr Leu Ser Ser Val Gly |
| | | Leu Ser Ser Val Leu Gly Thr Glu Glu Glu |
| | | Asp Glu Asp Glu Glu Tyr Glu Tyr Met Asn |
| | | Arg Arg Arg Arg His Ser Pro Pro His Pro |
| | | Pro Arg Pro Ser Ser Leu Glu Glu Leu Gly |
| | | Tyr Glu Tyr Met Asp Val Gly Ser Asp Leu |
| | | Ser Ala Ser Leu Gly Ser Thr Gln Ser Cys |
| | | Pro Leu His Pro Val Pro Ile Met Pro Thr |
| | | Ala Gly Thr Thr Pro Asp Glu Asp Tyr Glu |
| | | Tyr Met Asn Arg Gln Arg Asp Gly Gly Gly |
| | | Pro Gly Gly Asp Tyr Ala Ala Met Gly Ala |
| | | Cys Pro Ala Ser Glu Gln Gly Tyr Glu Glu |
| | | Met Arg Ala Phe Gln Gly Pro Gly His Gin |
| | | Ala Pro His Val His Tyr Ala Arg Leu Lys |
| | | Thr Leu Arg Ser Leu Glu Ala Thr Asp Ser |
| | | Ala Phe Asp Asn Pro Asp Tyr Trp His Ser |
| | | Arg Leu Phe Pro Lys Ala Asn Ala Gln Arg |
| | | Thr |

What is claimed is:

1. The use of a therapeutically effective amount of istiratumab in combination with an immunomodulatory agent to treat cancer in a human patient, where the immunomodulatory agent is selected from the group consisting of: (a) an agonistic anti-receptor antibody that immunospecifically binds human OX40, CD40, GITR, CD27, ICOS, or 4-1BB; (b) an antagonistic anti-receptor antibody that immunospecifically binds human CTLA-4, PD-1, PD-L1, TIM-3, BTLA, VISTA, LAG-3, KIR, CD47, CD25, B7-H3, or B7-H4; and (c) an anti-ligand antibody that blocks function of IL-6, IL-10, TGFβ, angiopoetin-2, VEGF, IL-17, IL-23, or TNFα.

2. The use of claim 1, wherein the cancer is selected from the group consisting of: colorectal cancer, melanoma, B-cell lymphoma and fibrosarcoma.

3. The use of claim 1 or 2, wherein the human patient does not have detectable free IGF-1 in serum prior to administration of the istiratumab to treat the cancer.

4. The use of any one of claims 1-3, wherein the PD-1 blocking antibody is nivolumab.

5. The use of any one of claims 1-4, wherein the cancer is melanoma.

6. The use of any one of claims 1-5, wherein the human patient does not have an active autoimmune condition prior to the administration of the istiratumab.

7. The use of any one of claims 1-6, wherein the istiratumab is administered once every two weeks to treat the cancer in the human patient.

8. The use of any one of claims 1-7, wherein the istiratumab is administered at a dose of 40 mg/kg or 2.8 g once every two weeks.

9. The use of any one of claims 1-8, wherein the PD-1 blocking antibody is administered once every 2 weeks at a dose of 3 mg/kg.

10. The use of any one of claims 1-3 and 5-8, wherein the anti-CTLA-4 antibody is ipilimumab or tremelimumab.

11. The use of any one of claims 1-9, wherein the anti-PD-1 antibody is selected from the group consisting of nivolumab, pembrolizumab, and pidilizumab.

12. The use of any one of claims 1-3 and 5-8, wherein the anti-PD-L1 antibody is selected from the group consisting of atezolizumab, durvalumab, and avelumab.

13. The use of claim 12, wherein the anti-PD-L1 antibody is atezolizumab.

14. The use of any one of claims 1-3 and 5-8, wherein the immunomodulatory agent is lirilumab.

15. The use of any one of claims 1-3, 5-8, and 10, wherein immunomodulatory agent is an anti-CTLA-4 antibody, and the cancer is melanoma.

16. The use of a therapeutically effective amount of istiratumab as an immune-oncology monotherapy to treat colorectal cancer, melanoma, B-cell lymphoma or fibrosarcoma cancer in a human patient without a diagnosed active autoimmune condition.

17. The use of claim 16, wherein the human patient has immune cells expressing IGF-1R and ErbB3.

18. The use of any one of claims 15-17, wherein the therapeutically effective amount of istiratumab is administered once every week or once every two weeks.

19. The use of any one of claims 15-18, wherein the therapeutically effective amount of istiratumab is a fixed dose of 2.8 g, 2.24 g, or 1.96 g or 40 mg/kg administered once every two weeks.

20. The use of an antineoplastic therapy consisting of a therapeutically effective amount of istiratumab in combination with an immunomodulatory agent selected from the group consisting of: an anti-PD-L1 antibody, an anti-CTLA-4 antibody and an anti-PD-1 antibody, to treat colorectal cancer, melanoma, B-cell lymphoma or fibrosarcoma cancer in a human patient having immune cells expressing IGF-1R and ErbB3 and without a diagnosed active autoimmune condition.

21. The use of claim 20, wherein the human patient has Treg cells expressing IGF-1R and ErbB3.

22. The use of claim 20 or 21, wherein the therapeutically effective amount of istiratumab is administered once every week or once every two weeks.

23. The use of any one of claims 20-22, wherein the therapeutically effective amount of istiratumab is a fixed dose of 2.8 g, 2.24 g, or 1.96 g or 40 mg/kg administered once every two weeks.

24. The use of any one of claims 16-23, wherein the patient has cancer cells that do not express at least one of IGF-1R and ErbB3.

25. The use of a therapeutically effective amount of istiratumab as an immune-oncology to treat a cancer that does not express at least one of IGF-1R and ErbB3 in a human patient without a diagnosed active autoimmune condition, and having immune cells expressing IGF-1R and ErbB3.

26. The use of claim 16, wherein the cancer is selected from the group consisting of: colorectal cancer, melanoma, B-cell lymphoma and fibrosarcoma cancer.

27. The use of any one of claims 24-26, wherein the therapeutically effective amount of istiratumab is administered as a monotherapy, without another immunomodulatory agent, once every week or once every two weeks.

28. The use of claim 27, wherein the therapeutically effective amount of istiratumab is a fixed dose of 2.8 g, 2.24 g, or 1.96 g or 40 mg/kg administered once every two weeks.

29. The use of an antineoplastic therapy consisting of istiratumab in combination with an immunomodulatory agent selected from the group consisting of: an anti-PD-L1 antibody, an anti-CTLA-4 antibody and an anti-PD-1 antibody, to treat a cancer that does not express at least one of IGF-1R and ErbB3 in a human patient without a diagnosed active autoimmune condition, and having immune cells expressing IGF-1R and ErbB3 in a human patient without a diagnosed active autoimmune condition.

30. The use of claim 29, wherein the cancer is selected from the group consisting of: colorectal cancer, melanoma, B-cell lymphoma and fibrosarcoma cancer.

31. The use of claim 29 or 30, wherein the therapeutically effective amount of istiratumab is administered as an antineoplastic therapy consisting of istiratumab and the immunomodulatory agent, once every week or once every two weeks.

32. The use of claim 31, wherein the therapeutically effective amount of istiratumab is a fixed dose of 2.8 g, 2.24 g, or 1.96 g or 40 mg/kg administered once every two weeks.
